(19) United States
(12) Patent Application Publication
(10) Pub. No.: US 2023/0119699 A1
(43) Pub. Date: Apr. 20, 2023

(54) DIAGNOSTIC METHODS USING SIRT1 EXPRESSION

(71) Applicant: Biorchestra Co., Ltd., Daejeon (KR)

(72) Inventors: Jin-Hyeob RYU, Daejeon (KR); Han Seok KOH, Daejeon (KR); Dae Hoon KIM, Daejeon (KR); Hyun Su MIN, Yuseong-gu (KR); Yu Na LIM, Daejeon (KR)

(73) Assignee: Biorchestra Co., Ltd., Daejeon (KR)

(21) Appl. No.: 17/906,175

(22) PCT Filed: Mar. 12, 2021
(86) PCT No.: PCT/IB2021/052098

§ 371 Date: Sep. 12, 2022

Related U.S. Application Data
(60) Provisional application No. 62/989,432, filed on Mar. 13, 2020.

(51) Int. Cl. C12N 15/113; C12Q 1/6883; A61P 25/28; A61K 47/54; A61K 47/60; A61K 9/107; A61P 29/00; A61K 47/18; A61K 47/20; A61K 47/64

(52) U.S. Cl. CPC C12N 15/113; C12Q 1/6883; A61P 25/28; A61K 47/549; A61K 47/60; A61K 9/1075; A61P 29/00; A61K 47/183; A61K 47/20; A61K 47/6455; C12Q 2600/158; C12N 2310/113

(57) ABSTRACT

The present disclosure relates to the use of SIRT1 expression to identify a subject that is conducive to treatment with a miR-485 inhibitor. In some aspects, the subject suffers from a disease or disorder associated with reduced SIRT1 expression. In some aspects, the SIRT1 expression is measured in the serum of the subject.

### CROSS-REFERENCE TO RELATED APPLICATION

This PCT application claims the priority benefit of U.S. Provisional Application No. 62/989,432, filed Mar. 13, 2020, which is incorporated herein by reference in its entirety.

### REFERENCE TO SEQUENCE LISTING SUBMITTED ELECTRONICALLY VIA EFS-WEB

The content of the electronically submitted sequence listing in ASCII text file (Name 4366_023PC01_Seqlisting_ST25.txt; Size: 77,549 bytes; and Date of Creation: Mar. 12, 2021) filed with the application is incorporated herein by reference in its entirety.

### FIELD OF THE DISCLOSURE

The present disclosure provides methods of identifying a subject responsive to a miR-485 inhibitor (e.g., polynucleotide encoding a nucleotide molecule comprising at least one miR-485 binding site) therapy and methods for the treatment of diseases and disorders associated with reduced SIRT 1 expression (e.g., neurodegenerative diseases and disorders, e.g., Alzheimer's disease).

### BACKGROUND OF THE DISCLOSURE

Sirtulin 1 (also known as NAD-dependent deacetylase sirtuin-1) is an enzyme that in humans is encoded by the SIRT1 gene. It belongs to a family of nicotinamide adenine dinucleotide (NAD)-dependent histone deacetylases and can deacetylate a variety of substrates. Rahman, S., et al., Cell Communication and Signaling 9:11 (2011). Accordingly, sirtulin 1 has been described as playing a role in a broad range of physiological functions, including control of gene expression, metabolism, and aging. And, abnormal sirtulin activity has been associated with certain human diseases. For instance, subjects with neurodegenerative disorders have been described as exhibiting low levels of sirtulin 1 activity.

Neurodegenerative disorders, such as Alzheimer's disease (AD), are common and growing cause of mortality and morbidity worldwide. It is estimated that by 2050, more than 100 million people worldwide will be affected by AD. Gaugler et al., Alzheimer's Dement 12(4): 459-509 (2016); Pan et al., Sci Adv 5(2) (2019). The costs of AD are estimated at more than 800 billion USD globally. Over the past two decades, investigators have been trying to develop compounds and antibodies that can inhibit Aβ production and aggregation, or, promote amyloid beta clearance. Unfortunately, these attempts have not achieved successful clinical benefits in large clinical trials with mild AD patients Panza et al., Nat Rev Neurol 15(2): 73-88 (2019).

Currently, there are no known cures for neurodegenerative disorders, let alone a methods of identifying a subject responsive to such a therapy. Available treatment options are generally limited to alleviating the various symptoms, as opposed to addressing the underlying causes of the disorders. Therefore, new and more effective approaches to treating neurodegenerative disorders and/or diagnosing such a therapy are highly desirable

### BRIEF SUMMARY OF THE DISCLOSURE

The present disclosure is directed to a method of identifying a subject responsive to a miR-485 inhibitor therapy comprising measuring a level of a SIRT1 protein and/or a SIRT1 gene in the subject. In some aspects, the subject is previously administered a compound that inhibits miR-485 (miRNA inhibitor). In some aspects, the method further comprises administering a compound that inhibits miR-485 (miRNA inhibitor). In some aspects, the subject has a disease or a condition associated with a decreased level of a SIRT1 protein and/or a SIRT1 gene. In some aspects, the miRNA inhibitor useful for the present method induces autophagy and/or treats or prevents inflammation.

Also disclosed herein includes a method of treating a disease or condition associated with an abnormal level of a SIRT1 protein and/or a SIRT1 gene in a subject in need thereof comprising administering to the subject a compound that inhibits miR-485 (miRNA inhibitor) and measuring a level of a SIRT1 protein and/or a SIRT1 gene in the subject. In some aspects, the level of the SIRT1 protein and/or SIRT1 gene is increased after the administration. In some aspects, the method further comprises administering a second dose of the miRNA inhibitor to the subject.

In some aspects, the level of a SIRT1 protein and/or a SIRT1 gene in the subject is increased at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% in a frontal cortex section compared to the level prior to the administration.

In some aspects, the level of a SIRT1 protein and/or a SIRT1 gene in the subject is increased at least about 30%, at least about 40%, at least about 50%, at least about 60%, at least about 70%, at least about 80%, at least about 90%, at least about 100%, at least about 110%, at least about 120%, at least about 130%, at least about 140%, or at least about 150% in a hippocampus section compared to the level prior to the administration.

In some aspects, the level of a SIRT1 protein and/or a SIRT1 gene in the subject is increased at least about 30%, at least about 40%, at least about 50%, at least about 60%, at least about 70%, at least about 80%, at least about 90%, or at least about 100% in serum compared to the level prior to the administration.

In some aspects, the level of a SIRT1 protein and/or a SIRT1 gene in the subject is measured within about 12 hours, about 24 hours, about 36 hours, or about 48 hours. In other aspects, the measuring is in serum of the subject. In some aspects, the serum is collected after the administration. In some aspects, the measuring is in the Cerebrospinal fluid (CSF) of the subject.

In some aspects, the miRNA inhibitor inhibits miR485-3p. In some aspects, the miR485-3p comprises 5′-GUCAUACACGGCUCUCCUCUCU-3′ (SEQ ID NO: 1). In some aspects, the miRNA inhibitor comprises a nucleotide sequence comprising 5′-UGUAUGA-3′ (SEQ ID NO: 2) and wherein the miRNA inhibitor comprises about 6 to about 30 nucleotides in length.

In some aspects, the miRNA inhibitor increases transcription of an SIRT1 gene and/or expression of a SIRT1 protein.

In some aspects, the miRNA inhibitor comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 5′ of the nucleotide sequence. In some aspects, the miRNA inhibitor comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 3′ of the nucleotide sequence.

In some aspects, the miRNA inhibitor has a sequence selected from the group consisting of: 5′-UGUAUGA-3′ (SEQ ID NO: 2), 5′-GUGUAUGA-3′ (SEQ ID NO: 3), 5′-CGUGUAUGA-3′ (SEQ ID NO: 4), 5′-CCGUGUAUGA-3′ (SEQ ID NO: 5), 5′-GCCGUGUAUGA-3′ (SEQ ID NO: 6), 5′-AGCCGUGUAUGA-3′ (SEQ ID NO: 7), 5′-GAGCCGUGUAUGA-3′ (SEQ ID NO: 8), 5′-AGAGCCGUGUAUGA-3′ (SEQ ID NO: 9), 5′-GAGAGCCGUGUAUGA-3′ (SEQ ID NO: 10), 5′-GGAGAGCCGUGUAUGA-3′ (SEQ ID NO: 11), 5′-AGGAGAGCCGUGUAUGA-3′ (SEQ ID NO: 12), 5′-GAGGAGAGCCGUGUAUGA-3′ (SEQ ID NO: 13), 5′-AGAGGAGAGCCGUGUAUGA-3′ (SEQ ID NO: 14), and 5′-GAGAGGAGAGCCGUGUAUGA-3′ (SEQ ID NO: 15).

In some aspects, the miRNA inhibitor has a sequence selected from the group consisting of: 5′-UGUAUGAC-3′ (SEQ ID NO: 16), 5′-GUGUAUGAC-3′ (SEQ ID NO: 17), 5′-CGUGUAUGAC-3′ (SEQ ID NO: 18), 5′-CCGUGUAUGAC-3′ (SEQ ID NO: 19), 5′-GCCGUGUAUGAC-3′ (SEQ ID NO: 20), 5′-AGCCGUGUAUGAC-3′ (SEQ ID NO: 21), 5′-GAGCCGUGUAUGAC-3′ (SEQ ID NO: 22), 5′-AGAGCCGUGUAUGAC-3′ (SEQ ID NO: 23), 5′-GAGAGCCGUGUAUGAC-3′ (SEQ ID NO: 24), 5′-GGAGAGCCGUGUAUGAC-3′ (SEQ ID NO: 25), 5′-AGGAGAGCCGUGUAUGAC-3′ (SEQ ID NO: 26), 5′-GAGGAGAGCCGUGUAUGAC-3′ (SEQ ID NO: 27), 5′-AGAGGAGAGCCGUGUAUGAC-3′ (SEQ ID NO: 28), 5′-GAGAGGAGAGCCGUGUAUGAC-3′ (SEQ ID NO: 29), and 5-AGAGAGGAGAGCCGUGUAUGAC-3′ (SEQ ID NO: 30).

In some aspects, the miRNA inhibitor has a sequence selected from the group consisting of: 5′-TGTATGA-3′ (SEQ ID NO: 62), 5′-GTGTATGA-3′ (SEQ ID NO: 63), 5′-CGTGTATGA-3′ (SEQ ID NO: 64), 5′-CCGTGTATGA-3′ (SEQ ID NO: 65), 5′-GCCGTGTATGA-3′ (SEQ ID NO: 66), 5′-AGCCGTGTATGA-3′ (SEQ ID NO: 67), 5′-GAGCCGTGTATGA-3′ (SEQ ID NO: 68), 5′-AGAGCCGTGTATGA-3′ (SEQ ID NO: 69), 5′-GAGAGCCGTGTATGA-3′ (SEQ ID NO: 70), 5′-GGAGAGCCGTGTATGA-3′ (SEQ ID NO: 71), 5′-AGGAGAGCCGTGTATGA-3′ (SEQ ID NO: 72), 5′-GAGGAGAGCCGTGTATGA-3′ (SEQ ID NO: 73), 5′-AGAGGAGAGCCGTGTATGA-3′ (SEQ ID NO: 74), 5′-GAGAGGAGAGCCGTGTATGA-3′ (SEQ ID NO: 75); 5′-TGTATGAC-3′ (SEQ ID NO: 76), 5′-GTGTATGAC-3′ (SEQ ID NO: 77), 5′-CGTGTATGAC-3′ (SEQ ID NO: 78), 5′-CCGTGTATGAC-3′ (SEQ ID NO: 79), 5′-GCCGTGTATGAC-3′ (SEQ ID NO: 80), 5′-AGCCGTGTATGAC-3′ (SEQ ID NO: 81), 5′-GAGCCGTGTATGAC-3′ (SEQ ID NO: 82), 5′-AGAGCCGTGTATGAC-3′ (SEQ ID NO: 83), 5′-GAGAGCCGTGTATGAC-3′ (SEQ ID NO: 84), 5′-GGAGAGCCGTGTATGAC-3′ (SEQ ID NO: 85), 5′-AGGAGAGCCGTGTATGAC-3′ (SEQ ID NO: 86), 5′-GAGGAGAGCCGTGTATGAC-3′ (SEQ ID NO: 87), 5′-AGAGGAGAGCCGTGTATGAC-3′ (SEQ ID NO: 88), 5′-GAGAGGAGAGCCGTGTATGAC-3′ (SEQ ID NO: 89), and 5′-AGAGAGGAGAGCCGTGTATGAC-3′ (SEQ ID NO: 90).

In some aspects, the sequence of the miRNA inhibitor is at least about 50%, at least about 55%, at least about 60%, at least about 65%, at least about 70%, at least about 75%, at least about 80%, at least about 85%, at least about 90%, or at least about 95% sequence identity to 5′-AGAGAGGAGAGCCGUGUAUGAC-3′ (SEQ ID NO: 30) or 5′-AGAGAGGAGAGCCGTGTATGAC-3′ (SEQ ID NO: 90). In certain aspects, the miRNA inhibitor has a sequence that has at least 90% similarity to 5′-AGAGAGGAGAGCCGUGUAUGAC-3′ (SEQ ID NO: 30) or 5′-AGAGAGGAGAGCCGTGTATGAC-3′ (SEQ ID NO: 90). In some aspects, the miRNA inhibitor comprises the nucleotide sequence 5′-AGAGAGGAGAGCCGUGUAUGAC-3′ (SEQ ID NO: 30) or 5′-AGAGAGGAGAGCCGTGTATGAC-3′ (SEQ ID NO: 90) with one substitution or two substitutions. In some aspects, the miRNA inhibitor comprises the nucleotide sequence 5′-AGAGAGGAGAGCCGUGUAUGAC-3′ (SEQ ID NO: 30) or 5′-AGAGAGGAGAGCCGTGTATGAC-3′ (SEQ ID NO: 90). In some aspects, the miRNA inhibitor comprises the nucleotide sequence 5′-AGAGAGGAGAGCCGUGUAUGAC-3′ (SEQ ID NO: 30).

In some aspects, the miRNA inhibitor comprises at least one modified nucleotide. In certain aspects, the at least one modified nucleotide is a locked nucleic acid (LNA), an unlocked nucleic acid (UNA), an arabino nucleic acid (ABA), a bridged nucleic acid (BNA), and/or a peptide nucleic acid (PNA).

In some aspects, the miRNA inhibitor comprises a backbone modification. In certain aspects, the backbone modification is a phosphorodiamidate morpholino oligomer (PMO) and/or phosphorothioate (PS) modification.

In some aspects, the miRNA inhibitor is delivered in a delivery agent. In certain aspects, the delivery agent comprises a micelle, an exosome, a lipidoid, a liposome, a lipoplex, a lipid nanoparticle, an extracellular vesicle, a synthetic vesicle, a polymeric compound, a peptide, a protein, a cell, a nanoparticle mimic, a nanotube, a conjugate, a viral vector, or combinations thereof.

In some aspects, the delivery agent comprises a cationic carrier unit comprising

[WP]-L1-[CC]-L2-[AM] (formula I)

or

[WP]-L1-[AM]-L2-[CC] (formula II)

wherein
WP is a water-soluble biopolymer moiety;
CC is a cationic carrier moiety;
AM is an adjuvant moiety; and,
L1 and L2 are independently optional linkers.

In some aspects, the miRNA inhibitor and the cationic carrier unit are capable of associating with each other to form a micelle when mixed together. In certain aspects, the association is via a covalent bond. In some aspects, the association is via a non-covalent bond. In some aspects, the miRNA inhibitor interacts with the cationic carrier unit via an ionic bond. In some aspects, the cationic carrier unit is capable of protecting the miRNA inhibitor from enzymatic degradation.

In some aspects, the water-soluble polymer comprises poly(alkylene glycols), poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyglycerol, polyphosphazene, polyoxazolines (“POZ”) poly(N-acryloylmorpholine), or any combinations thereof. In other aspects, the water-soluble polymer comprises polyethylene glycol (“PEG”), polyglycerol, or poly(propylene glycol) (“PPG”).

In some aspects, the water-soluble polymer comprises:

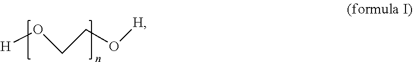

wherein n is 1-1000.

In certain aspects, the n is at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, or at least about 141. In further aspects, the n is about 80 to about 90, about 90 to about 100, about 100 to about 110, about 110 to about 120, about 120 to about 130, about 140 to about 150, about 150 to about 160.

In some aspects, the water-soluble polymer is linear, branched, or dendritic.

In some aspects, the cationic carrier moiety comprises one or more basic amino acids. In certain aspects, the cationic carrier moiety comprises at least three, at least four, at least five, at least six, at least seven, at least eight, at least nine, at least ten, at least 11, at least 12, at least 13, at least 14, at last 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 basic amino acids. In certain aspects, the cationic carrier moiety comprises about 30 to about 50 basic amino acids.

In some aspects, the basic amino acid comprises arginine, lysine, histidine, or any combination thereof. In some aspects, the cationic carrier moiety comprises about 40 lysine monomers.

In some aspects, the adjuvant moiety is capable of modulating an immune response, an inflammatory response, and/or a tissue microenvironment. In certain aspects, the adjuvant moiety comprises an imidazole derivative, an amino acid, a vitamin, or any combination thereof.

In some aspects, the adjuvant moiety comprises:

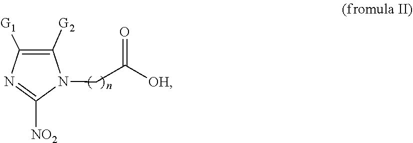

wherein each of G1 and G2 is H, an aromatic ring, or 1-10 alkyl, or G1 and G2 together form an aromatic ring, and wherein n is 1-10.

In some aspects, the adjuvant moiety comprises nitroimidazole. In certain aspects, the adjuvant moiety comprises metronidazole, tinidazole, nimorazole, dimetridazole, pretomanid, ornidazole, megazol, azanidazole, benznidazole, or any combination thereof.

In some aspects, the adjuvant moiety comprises an amino acid.

In some aspects, the adjuvant moiety comprises

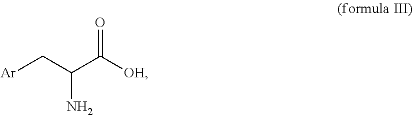

wherein Ar is

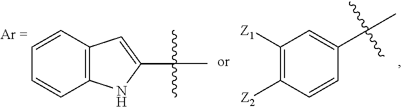

and

wherein each of Z1 and Z2 is H or OH.

In some aspects, the adjuvant moiety comprises a vitamin. In certain aspects, the vitamin comprises a cyclic ring or cyclic hetero atom ring and a carboxyl group or hydroxyl group.

In some aspects, the vitamin comprises:

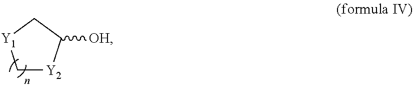

wherein each of Y1 and Y2 is C, N, O, or S, and wherein n is 1 or 2.

In some aspects, the vitamin is selected from the group consisting of vitamin A, vitamin B1, vitamin B2, vitamin B3, vitamin B6, vitamin B7, vitamin B9, vitamin B12, vitamin C, vitamin D2, vitamin D3, vitamin E, vitamin M, vitamin H, and any combination thereof. For example, the vitamin can be vitamin B3.

In some aspects, the adjuvant moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, or at least about 20 vitamin B3. In certain aspects, the adjuvant moiety comprises about 10 vitamin B3.

In some aspects, the delivery agent comprises a water-soluble biopolymer moiety with about 120 to about 130 PEG units, a cationic carrier moiety comprising a poly-lysine with about 30 to about 40 lysines, and an adjuvant moiety with about 5 to about 10 vitamin B3.

In some aspects, a disease or a condition that can be treated with the present disclosure comprises Alzheimer's disease. In certain aspects, the disease or condition comprises autism spectrum disorder, mental retardation, seizure, stroke, Parkinson's disease, spinal cord injury, or any combination thereof.

In some aspects, a delivery agent used to deliver a miRNA inhibitor described herein is a micelle. In certain aspects, the micelle comprises (i) about 100 to about 200 PEG units, (ii) about 30 to about 40 lysines, each with an amine group, (iii) about 15 to about 20 lysines, each with a thiol group, and (iv) about 30 to about 40 lysines, each linked to vitamin B3. In some aspects, the micelle comprises (i) about 120 to about 130 PEG units, (ii) about 32 lysines, each with an amine group, (iii) about 16 lysines, each with a thiol group, and (iv) about 32 lysines, each linked to vitamin B3.

In some aspects, a targeting moiety is further linked to the PEG units of the micelles described above. In some aspects, the targeting moiety is a LAT 1 targeting ligand. In some aspects, the targeting moiety is phenyl alanine.

### BRIEF DESCRIPTION OF THE DRAWINGS/FIGURES

FIGS. 1A, 1i, 1C, and 1D shows that SIRT1 expression is decreased in Alzheimer's disease subjects. FIG. 1A provides a comparison of representative SIRT1 protein expression in precentral gyrus tissues from normal (i.e., subjects without AD) and AD patients (n=6 for each group). FIG. 1B provides a quantitative comparison of the results shown in FIG. 1A. SIRT1 bands were analyzed by densitometry and normalized to β-actin. Relative levels of SIRT1 protein are shown from control (n=6) or AD precentral gyrus (n=6) tissues. FIG. 1C provides a comparison of SIRT1 mRNA expression in in 6 mo-old wild-type (WT) (n=4), 6 mo-old 5XFAD (n=3), 11 mo-old wild-type (WT), and 11 mo-old 5XFAD mice (n=3). Comparative analyzes were performed for mice at the same ages. FIG. 1D provides a comparison of SIRT1 mRNA expression in 5XFAD mice by age. Each age group's 5XFAD expression was normalized to WT. In FIGS. 1B, 1C, and 1D, the bars represent mean±SD.

FIGS. 2A and 2B provide comparison of miR485-3p and miR485-5p expression in normal (i.e., subjects without AD) and AD patients, respectively.

FIG. 3 provides a comparison of relative levels of mouse miR485-3p expression in primary cortical neurons transfected with either the control oligonucleotide (“1”) or the miR485 inhibitor (“2”). The graph on the left shows miR485-3p expression at 3 hours after transfection with miR485-3p ASO. The graph on the right shows expression at 6 hours after transfection with miR485-3p ASO. In each of the graphs, the left bar represents the control group and the right bar represents the miR-485 inhibitor transfected group.

FIGS. 4A and 4B show that miR-485 inhibitors can increase SIRT1 and PGC-1α expression. FIG. 4A provides western blot results showing SIRT1 and PGC-1α protein expression in mouse primary cortical neurons transfected with miR-control, miR485-3p (“miR485-3p mimic”), or miR-485 inhibitor (“miR485-3p ASO”). FIG. 4B provides a quantitative comparison of the results shown in FIG. 4A.

FIGS. 5A, 5B, and 5C show that miR-485-3p mimic functionally binds to the 3′ UTR of SIRT1. FIG. 5A is a schematic representation of the wild type (WT) or mutant form (MT) in SIRT1 3′-UTR showing the putative miR-485-3p target site. FIG. 5B provides a comparison of the relative luciferase activity in HEK293T cells co-transfected with SIRT1 3′-UTR WT or MT reporter constructs and either miR-control (“1”) or miR-485-3p mimic (“2”) for 48 hours. At least three independent experiments were performed. FIG. 5C provides a comparison of the relative binding of miR485-3p mimic onto 3′ UTR of SIRT1 harboring mutant seed region (“MT SIRT1”) compared to WT 3′ UTR of SIRT1 (“WT SIRT1”).

FIGS. 6A, 6B, 6C, 6D, 6E, 6F, and 6G show that the miR-485 inhibitor reduces Aβ deposition and alters APP processing. FIG. 6A provides the schedule of miR-485 inhibitor ICV injections in 10 mo-old 5XFAD mice. FIG. 6B provides representative images of immunohistochemical staining for Aβ (6E10) in the cortex and hippocampal DG region from control (n=5) and miR-485 inhibitor (“miR485-3p ASO”) (n=5) injected 5XFAD mice. FIG. 6C provides a quantitative comparison (mean number of Aβ plaques per mm²) of the results shown in FIG. 6B. FIG. 6D provides immunoblot for insoluble Ab fractions in control (n=3) or miR-485 inhibitor (“miR485-3p ASO”) (n=3) injected 10 mo-old 5XFAD mice. FIG. 6E provides a quantitative comparison of the data shown in FIG. 6D. FIG. 6F provides western blot showing APP, sAPPβ, sAPPα, β-CTFs, BACE1, Adam10 and SIRT1 protein expression in control (n=3) or miR-485 inhibitor (“miR485-3p ASO”) (n=3) injected 10 mo-old 5XFAD mice. FIG. 6G provides a quantitative comparison (i.e., relative levels) of the data shown in FIG. 6F.

FIGS. 7A, 7B, 7C, 7D, 7E, 7F, 7G, and 7H show that miR-485 inhibitor enhances phagocytosis of Aβ both in vitro and in vivo by increasing CD36 expression. FIG. 7A provides an immunohistochemistry analysis of Iba1 (microglia) (left column) and β-amyloid 1-16 (6E10, to detect Aβ plaque) (middle column) on coronal sections of control (n=11 images from five mice) (top row) or miR485-3p ASO (“miR485-3p ASO”) (bottom row) (n=11 images from five mice) injected 5XFAD mice. The right column provides an overlay of the images shown in the left and middle columns. FIG. 7B provides a quantitative comparison (mean number of Iba1+Aβ⁺ cells per mm²) of the data shown in FIG. 7A. FIG. 7C provides representative images of ThS staining to Aβ plaque in hippocampus and cortex of control (n=7 images from three mice) or miR-485 inhibitor (“miR485-3p ASO”) (n=7 images from three mice) administrated mice. FIG. 7D provides a quantitative comparison of the data shown in FIG. 7C. FIG. 7E provides an immunohistochemistry analysis showing the uptake of Aβ plaques (Aβ1-42) by the primary microglial cells (Iba1+) in mouse primary mixed glial cells transfected and/or treated with one of the following: (i) transfected with control oligonucleotide (top row), (ii) treated with oAβ(1-42) (1 μM) (middle row), or (iii) transfected with miR-485 inhibitor (“miR485-3p ASO”) and treated with oAβ(1-42) (1 μM) (bottom row). FIG. 7F provides immunohistochemistry analysis of histological brain sections from control (n=6) (top row) or miR-485 inhibitor (“miR485-3p ASO”) (n=6) (bottom row) injected 5XFAD mice using anti-Iba1, anti-CD68 (phagosome) and anti-β-amyloid 1-16 (6E10). FIG. 7G provides a quantitative comparison (mean number of Iba1*Aβ⁺ CD68⁺ cells per mm²) of the results shown in FIG. 7F. FIG. 7H provides a comparison of Aβ levels in supernatant of BV2 microglia cells transfected with either control oligonucleotide or miR-485 inhibitor (“miR485-3p ASO”) and further treated with oAβ(1-42) (1 μM). Supernatant was collected after 4 hours of treatment and analyzed using ELISA.

FIGS. 8A, 8B, 8C, 8D, and 8E show that miR-485 inhibitor can increase CD36 expression. FIG. 8A provides a comparison of the relative levels of CD36 protein expression in control (n=3) or miR-485 inhibitor (“miR485-3p ASO”) (n=3) injected 10 mo-old 5XFAD mice. FIG. 8B provides a quantitative comparison of the results shown in FIG. 8A. FIG. 8C provides an immunohistochemistry analysis of histological brain sections from the control (top row) or miR-485 inhibitor (“miR485-3p ASO”) (bottom row) treated 5XFAD mice using anti-Iba1 and anti-b-amyloid 1-16 (6E10) antibodies. The first column shows the expression of CD36+ cells. The middle column shows the expression of Iba1+ cells. The right column provides an overlay of the cells shown in the left and middle columns. FIG. 8D provides cell surface expression of CD36 as measured by flow cytometry using Alexa488-conjugated anti-CD36 antibody in control (n=3), miR485-3p mimic (top graph), or miR-485 inhibitor (“miR485-3p ASO”) (n=3) (bottom graph) transfected primary mixed glial cells. FIG. 8E provides a quantitative comparison (relative mean fluorescence intensity) of the results shown in FIG. 8D.

FIG. 9 shows that miR-485-3p mimic can functionally bind to the 3′ UTR of CD36. Relative luciferase activity was measured in HEK293T cells co-transfected with CD36 3′-UTR WT (first two bars) or MT reporter constructs (last two bars) and either miR-control (“1”) or miR-485 mimic (“2”) for 48 h.

FIG. 10 shows that miR-485 inhibitor can promote increased Aβ phagocytosis through CD36 regulation. Aβ levels in supernatant of BV2 microglia cells transfected with either control oligonucleotide or miR-485 inhibitor (“miR485-3p ASO”) and further treated with oAβ(1-42) (1 μM). Where indicated, a blocking anti-CD36 antibody was also added. Supernatant was collected after 4 hours of treatment and analyzed using ELISA.

FIGS. 11A, 11B, 11C, 11D, 11E, 11F, 11G, and 11H show that miR-485 inhibitor can reduce neuroinflammation in glial cells. FIG. 11A provides western blot analysis showing SIRT1, NF-κB (p65), TNF-α and IL-1b protein expression in control or miR-485 inhibitor (“miR485-3p ASO”) transfected primary mixed glial cells treated with oAβ(1-42) (1 μM) for 3 (top immunoblot) or 6 hours (bottom immunoblot). “(1)” corresponds to cells transfected with the control oligonucleotide alone. “(2)” corresponds to cells treated with oAβ(1-42) alone. “(3)” corresponds to cells transfected with the miR-485 inhibitor and treated with oAβ(1-42). FIG. 11B provides a quantitative comparison of the results provided in FIG. 11A. FIG. 11C provides immunoblot detection of Iba1, NF-κB (p65), TNF-α and IL-1b protein in control (n=3) or miR-485 inhibitor (“miR485-3p ASO”) (n=3) injected 10 mo-old 5XFAD mice. Results from two independent experiments are shown (i.e., Set #1 and Set #2). FIG. 11D provides a quantitative comparison of the results shown in FIG. 11C. FIG. 11E provides an immunohistochemistry analysis of Iba1 and TNF-α expression in the control (n=11 images from five mice) or miR-485 inhibitor (“miR485-3p ASO”) (n=11 images from five mice) injected 5XFAD mice. FIG. 11F provides a quantitative comparison (mean number of Iba1 and TNF-α-stained cells per mm²) of the results shown in FIG. 11E. FIG. 11G provides an immunohistochemistry analysis of Iba1 and TNF-α expression in the control (n=11 images from five mice) or miR-485 inhibitor (“miR485-3p ASO”) (n=11 images from five mice) injected 5XFAD mice. FIG. 11H provides a quantitative comparison (mean number of Iba1 and IL-1β-stained cells per mm²) of the results shown in FIG. 11G.

FIGS. 12A, 12B, 12C, 12D, 12E, and 12F show that miR-485 inhibitor ameliorates neuronal loss and increases post-synapse. FIG. 12A provides immunoblot showing NeuN and cleaved caspase 3 protein expression in the hippocampus (left) and cortex (right) of control (n=3) or miR-485 inhibitor (“miR485-3p ASO”) (n=5) injected 10 mo-old 5XFAD mice. FIG. 12B provides a quantitative comparison of the results provided in FIG. 12A. FIG. 12C provides an immunohistochemistry analysis showing NeuN and cleaved caspase-3 expression in coronal brain sections from control (n=4) or miR-485 inhibitor (“miR485-3p ASO”) (n=5) injected 10 mo-old 5XFAD mice. FIG. 12D provides a quantitative comparison (mean number of NeuN and cleaved caspase-3-stained cells per mm²) of the results shown in FIG. 12C. FIG. 12E provides immunoblot analysis of PSD-95 protein expression in control (n=3) or miR-485 inhibitor (“miR485-3p ASO”) (n=5) injected 10 mo-old 5XFAD mice. Results from two independent experiments are shown (i.e., Set #1 and Set #2). FIG. 12F provides a quantitative comparison of the results shown in FIG. 12E.

FIGS. 13A and 13B show that miR-485 inhibitor improves cognitive decline in 5XFAD mice. FIGS. 13A and 13B provides the results from the Y-maze and passive avoidance tests, respectively for mice (10 mo-old 5XFAD mice) treated with either the control oligonucleotide or the miR-485 inhibitor (“miR485-3p ASO”). Average alternation (%) for control or miR485-3p injected 5XFAD mice and total entry number into each arm on Y-maze. Average step through latency and time in dark compartment in seconds for control or miR485-3p injected 5XFAD mice on passive avoidance test.

FIG. 14 provides a schematic diagram of possible non-limiting different means by which a miR-485 inhibitor can treat Alzheimer's disease as demonstrated through 5XFAD mice. miR-485 inhibitor in 5XFAD can increase SIRT1 expression in neurons. SIRT1 in turn can reduce amyloid beta production through regulation of amyloid production enzymes. Also, miR-485 inhibitor can enhance CD36 expression and phagocytosis of Aβ plaque in glial cells. At the same time, miR-485 inhibitor can induce SIRT1 expression and reduce neuroinflammation and neuronal damage.

FIGS. 15A, 15B, and 15C show that the expression of SIRT1 and PGC-1α increases in mouse brain cortex after a single intravenous administration of a miR-485 inhibitor. FIG. 15A provides the expression level of SIRT1 (left graph) and PGC-1α (right graph) at 6, 24, 48, and 72 hours after administration of the miR-485 inhibitor (100 μg/mouse). FIGS. 15B and 15C show the positive correlation between SIRT1 and PGC-1α expression, respectively, and time over a course of about 50 hours. In each of FIGS. 15A, 15B, and 15C, SIRT1 and PGC-1α expression level are shown normalized to the control (i.e., expression level in mice not treated with the miR-485 inhibitor). The percent values provided in FIG. 15A represent the average percent increase in SIRT1 and PGC-1α expression over the control at 48 hours post miR-485 inhibitor administration. In FIG. 15A, the p values provided represent the p value of t test. In FIGS. 15B and 15C, the p values provided represent the p value of Pearson's correlation. “C.C” represents the correlation coefficient of Pearson's correlation.

FIGS. 16A, 16B, and 16C show that the expression of SIRT1 and PGC-1α increases in the hippocampus of mouse brain after a single intravenous administration of a miR-485 inhibitor. FIG. 16A provides the expression level of SIRT1 (left graph) and PGC-1α (right graph) at 6, 24, 48, and 72 hours after administration of the miR-485 inhibitor (100 μg/mouse).

FIGS. 16B and 16C show the positive correlation between SIRT1 and PGC-1α expression, respectively, and time over a course of about 25 hours. In each of FIGS. 16A, 16B, and 16C, SIRT1 and PGC-1α expression level are shown normalized to the control (i.e., expression level in mice not treated with the miR-485 inhibitor). The percent values provided in FIG. 16A represent the average percent increase in SIRT1 and PGC-1α expression over the control at 24 hours post miR-485 inhibitor administration. In FIG. 16A, the p values provided represent the p value of t test. In FIGS. 16B and 16C, the p values provided represent the p value of Pearson's correlation. “C.C” represents the correlation coefficient of Pearson's correlation.

FIGS. 17A and 17B show that the expression of CD36 increases in mouse brain after a single intravenous administration of a miR-485 inhibitor (100 μg/mouse). FIG. 17A provides the expression level of CD36 at 24, 48, 72, and 120 hours after administration of the miR-485 inhibitor (100 μg/mouse). FIG. 17B shows the positive correlation between CD36 expression and time over a course of about 80 hours. In each of FIGS. 17A and 17B, CD36 expression is shown normalized to the control (i.e., expression level in mice not treated with the miR-485 inhibitor). The percent value provided in FIG. 17A represents the average percent increase in CD36 expression over the control at 48 hours post miR-485 inhibitor administration. In FIG. 17A, the p values provided represent the p value of t test. In FIG. 17B, the p value provided represents the p value of Pearson's correlation. “CC” represents the correlation coefficient of Pearson's correlation.

FIG. 18A shows the level of SIRT1 protein expression in the serum of mice after administration of the miR-485 inhibitor (“miR485-3p ASO”). FIG. 18B shows the level of PGC-1α protein expression in the serum of mice after administration of the miR-485 inhibitor (“miR485-3p ASO”). The X axis for FIGS. 18A and 18B show the time after the administration, and the y axis shows the level of protein normalized to control. The miR-485 inhibitor (“miR485-3p ASO”) is administered at a single IV dose of 100 μg/mouse. FIG. 18C shows the level of SIRT1 protein expression of FIG. 18A in line graph. FIG. 18D shows the level of PGC-1α protein expression in FIG. 18B in line graph.

FIG. 19 shows an exemplary architecture of a carrier unit of the present disclosure. The example presented includes a cationic carrier moiety, which can interact electrostatically with anionic payloads, e.g., nucleic acids such as antisense oligonucleotides targeting a gene, e.g., miRNA (antimirs). In some aspects, AM can be located between WP and CC. The CC and AM components are portrayed in a linear arrangement for simplicity. However, as described herein, in some aspects, CC and AM can be arranged in a scaffold fashion.

### DETAILED DESCRIPTION OF THE DISCLOSURE

The present disclosure is directed to diagnosing a subject who is responsive to a miR-485 inhibitor therapy, comprising measuring a level of a SIRT1 protein and/or a SIRT1 gene in the subject. The miR-485 inhibitor comprises a nucleotide molecule that comprises at least one miR-485 binding site, wherein the nucleotide molecule does not encode a protein. In some aspects, the miRNA binding site or sites can bind to endogenous miR-485, which inhibits and/or reduces the expression level of an endogenous SIRT1 protein and/or a SIRT1 gene.

Before the present disclosure is described in greater detail, it is to be understood that this disclosure is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order which is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, ‘a’ represents adenine, ‘c’ represents cytosine, ‘g’ represents guanine, ‘t’ represents thymine, and ‘u’ represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAVrh.74, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers). In some aspects, an “AAV” includes a derivative of a known AAV. In some aspects, an “AAV” includes a modified or an artificial AAV.

The terms “administration,” “administering,” and grammatical variants thereof refer to introducing a composition, such as a miRNA inhibitor of the present disclosure, into a subject via a pharmaceutically acceptable route. The introduction of a composition, such as a micelle comprising a miRNA inhibitor of the present disclosure, into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “associated with” refers to a close relationship between two or more entities or properties. For instance, when used to describe a disease or condition that can be treated with the present disclosure (e.g., disease or condition associated with an abnormal level of a SIRT1 protein and/or SIRT1 gene), the term “associated with” refers to an increased likelihood that a subject suffers from the disease or condition when the subject exhibits an abnormal expression of the protein and/or gene. In some aspects, the abnormal expression of the protein and/or gene causes the disease or condition. In some aspects, the abnormal expression does not necessarily cause but is correlated with the disease or condition. Non-limiting examples of suitable methods that can be used to determine whether a subject exhibits an abnormal expression of a protein and/or gene associated with a disease or condition are provided elsewhere in the present disclosure.

As used herein, the term “abnormal level” refers to a level (expression and/or activity) that differs (e.g., increased) from a reference subject who does not suffer from a disease or condition described herein (e.g., neurodegenerative disease and disorders, e.g., Alzheimer's disease). In some aspects, an abnormal level (e.g., SIRT1) refers to a level that is decreased by at least about 0.1-fold, at least about 0.2-fold, at least about 0.3-fold, at least about 0.4-fold, at least about 0.5-fold, at least about 0.6-fold, at least about 0.7-fold, at least about 0.8-fold, at least about 0.9-fold, at least about 1-fold, at least about 2-fold, at least about 3-fold, at least about 4-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, at least about 750-fold, or at least about 1,000-fold or more compared to the corresponding level in a reference subject (e.g., subject who does not suffer from a disease or condition described herein).

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least 70% identical, at least 80% identical, at least 90% identical, or at least 95% identical to one another. In some aspects, two or more sequences are said to be “highly conserved” if they are about 70% identical, about 80% identical, about 90% identical, about 95%, about 98%, or about 99% identical to one another. In some aspects, two or more sequences are said to be “conserved” if they are at least 30% identical, at least 40% identical, at least 50% identical, at least 60% identical, at least 70% identical, at least 80% identical, at least 90% identical, or at least 95% identical to one another. In some aspects, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence can apply to the entire length of a polynucleotide or polypeptide or can apply to a portion, region or feature thereof.

The term “derived from,” as used herein, refers to a component that is isolated from or made using a specified molecule or organism, or information (e.g., amino acid or nucleic acid sequence) from the specified molecule or organism. For example, a nucleic acid sequence that is derived from a second nucleic acid sequence can include a nucleotide sequence that is identical or substantially similar to the nucleotide sequence of the second nucleic acid sequence. In the case of nucleotides or polypeptides, the derived species can be obtained by, for example, naturally occurring mutagenesis, artificial directed mutagenesis or artificial random mutagenesis. The mutagenesis used to derive nucleotides or polypeptides can be intentionally directed or intentionally random, or a mixture of each. The mutagenesis of a nucleotide or polypeptide to create a different nucleotide or polypeptide derived from the first can be a random event (e.g., caused by polymerase infidelity) and the identification of the derived nucleotide or polypeptide can be made by appropriate screening methods, e.g., as discussed herein. In some aspects, a nucleotide or amino acid sequence that is derived from a second nucleotide or amino acid sequence has a sequence identity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% to the second nucleotide or amino acid sequence, respectively, wherein the first nucleotide or amino acid sequence retains the biological activity of the second nucleotide or amino acid sequence.

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide.

The terms “complementary” and “complementarity” refer to two or more oligomers (i.e., each comprising a nucleobase sequence), or between an oligomer and a target gene, that are related with one another by Watson-Crick base-pairing rules. For example, the nucleobase sequence “T-G-A (5′→3′),” is complementary to the nucleobase sequence “A-C-T (3→5′).” Complementarity can be “partial,” in which less than all of the nucleobases of a given nucleobase sequence are matched to the other nucleobase sequence according to base pairing rules. For example, in some aspects, complementarity between a given nucleobase sequence and the other nucleobase sequence can be about 70%, about 75%, about 80%, about 85%, about 90%, or about 95%. Accordingly, in certain aspects, the term “complementary” refers to at least about 80%, at least about 85%, at least about 90%, at least about 91%, at least about 92%, at least about 93%, at least about 94%, at least about 95%, at least about 96%, at least about 97%, at least about 98%, or at least about 99% match or complementarity to a target nucleic acid sequence (e.g., miR-485 nucleic acid sequence). Or, there can be “complete” or “perfect” (100%) complementarity between a given nucleobase sequence and the other nucleobase sequence to continue the example. In some aspects, the degree of complementarity between nucleobase sequences has significant effects on the efficiency and strength of hybridization between the sequences.

The term “downstream” refers to a nucleotide sequence that is located 3′ to a reference nucleotide sequence. In certain aspects, downstream nucleotide sequences relate to sequences that follow the starting point of transcription. For example, the translation initiation codon of a gene is located downstream of the start site of transcription.

The terms “excipient” and “carrier” are used interchangeably and refer to an inert substance added to a pharmaceutical composition to further facilitate administration of a compound, e.g., a miRNA inhibitor of the present disclosure.

The term “expression,” as used herein, refers to a process by which a polynucleotide produces a gene product, e.g., RNA or a polypeptide. It includes without limitation transcription of the polynucleotide into micro RNA binding site, small hairpin RNA (shRNA), small interfering RNA (siRNA), or any other RNA product. It includes, without limitation, transcription of the polynucleotide into messenger RNA (mRNA), and the translation of mRNA into a polypeptide. Expression produces a “gene product.” As used herein, a gene product can be, e.g., a nucleic acid, such as an RNA produced by transcription of a gene. As used herein, a gene product can be either a nucleic acid, RNA or miRNA produced by the transcription of a gene, or a polypeptide which is translated from a transcript. Gene products described herein further include nucleic acids with post transcriptional modifications, e.g., polyadenylation or splicing, or polypeptides with post translational modifications, e.g., phosphorylation, methylation, glycosylation, the addition of lipids, association with other protein subunits, or proteolytic cleavage.

As used herein, the term “homology” refers to the overall relatedness between polymeric molecules, e.g. between nucleic acid molecules. Generally, the term “homology” implies an evolutionary relationship between two molecules. Thus, two molecules that are homologous will have a common evolutionary ancestor. In the context of the present disclosure, the term homology encompasses both to identity and similarity.

In some aspects, polymeric molecules are considered to be “homologous” to one another if at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of the monomers in the molecule are identical (exactly the same monomer) or are similar (conservative substitutions). The term “homologous” necessarily refers to a comparison between at least two sequences (e.g., polynucleotide sequences).

In the context of the present disclosure, substitutions (even when they are referred to as amino acid substitution) are conducted at the nucleic acid level, i.e., substituting an amino acid residue with an alternative amino acid residue is conducted by substituting the codon encoding the first amino acid with a codon encoding the second amino acid.

As used herein, the term “identity” refers to the overall monomer conservation between polymeric molecules, e.g., between polynucleotide molecules. The term “identical” without any additional qualifiers, e.g., polynucleotide A is identical to polynucleotide B, implies the polynucleotide sequences are 100% identical (100% sequence identity). Describing two sequences as, e.g., “70% identical,” is equivalent to describing them as having, e.g., “70% sequence identity.”

Calculation of the percent identity of two polypeptide or polynucleotide sequences, for example, can be performed by aligning the two sequences for optimal comparison purposes (e.g., gaps can be introduced in one or both of a first and a second polypeptide or polynucleotide sequences for optimal alignment and non-identical sequences can be disregarded for comparison purposes). In certain aspects, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding amino acid positions, or bases in the case of polynucleotides, are then compared.

When a position in the first sequence is occupied by the same amino acid or nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs that can be used to align different sequences (e.g., polynucleotide sequences) are available from various sources. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

As used herein, the terms “isolated,” “purified,” “extracted,” and grammatical variants thereof are used interchangeably and refer to the state of a preparation of desired composition of the present disclosure, e.g., a miRNA inhibitor of the present disclosure, that has undergone one or more processes of purification. In some aspects, isolating or purifying as used herein is the process of removing, partially removing (e.g., a fraction) of a composition of the present disclosure, e.g., a miRNA inhibitor of the present disclosure from a sample containing contaminants.

In some aspects, an isolated composition has no detectable undesired activity or, alternatively, the level or amount of the undesired activity is at or below an acceptable level or amount. In other aspects, an isolated composition has an amount and/or concentration of desired composition of the present disclosure, at or above an acceptable amount and/or concentration and/or activity. In other aspects, the isolated composition is enriched as compared to the starting material from which the composition is obtained. This enrichment can be by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%, at least about 99.9999%, or greater than 99.9999% as compared to the starting material.

In some aspects, isolated preparations are substantially free of residual biological products. In some aspects, the isolated preparations are 100% free, at least about 99% free, at least about 98% free, at least about 97% free, at least about 96% free, at least about 95% free, at least about 94% free, at least about 93% free, at least about 92% free, at least about 91% free, or at least about 90% free of any contaminating biological matter. Residual biological products can include abiotic materials (including chemicals) or unwanted nucleic acids, proteins, lipids, or metabolites.

The term “linked” as used herein refers to a first amino acid sequence or polynucleotide sequence covalently or non-covalently joined to a second amino acid sequence or polynucleotide sequence, respectively. The first amino acid or polynucleotide sequence can be directly joined or juxtaposed to the second amino acid or polynucleotide sequence or alternatively an intervening sequence can covalently join the first sequence to the second sequence. The term “linked” means not only a fusion of a first polynucleotide sequence to a second polynucleotide sequence at the 5′-end or the 3′-end, but also includes insertion of the whole first polynucleotide sequence (or the second polynucleotide sequence) into any two nucleotides in the second polynucleotide sequence (or the first polynucleotide sequence, respectively). The first polynucleotide sequence can be linked to a second polynucleotide sequence by a phosphodiester bond or a linker. The linker can be, e.g., a polynucleotide.

A “miRNA inhibitor,” as used herein, refers to a compound that can decrease, alter, and/or modulate miRNA expression, function, and/or activity. The miRNA inhibitor can be a polynucleotide sequence that is at least partially complementary to the target miRNA nucleic acid sequence, such that the miRNA inhibitor hybridizes to the target miRNA sequence. For instance, in some aspects, a miR-485 inhibitor of the present disclosure comprises a nucleotide sequence encoding a nucleotide molecule that is at least partially complementary to the target miR-485 nucleic acid sequence, such that the miR-485 inhibitor hybridizes to the miR-485 sequence. In further aspects, the hybridization of the miR-485 to the miR-485 sequence decreases, alters, and/or modulates the expression, function, and/or activity of miR-485 (e.g., hybridization results in an increase in the expression of SIRT1 protein and/or SIRT1 gene).

The terms “miRNA,” “miR,” and “microRNA” are used interchangeably and refer to a microRNA molecule found in eukaryotes that is involved in RNA-based gene regulation. The term will be used to refer to the single-stranded RNA molecule processed from a precursor. In some aspects, the term “antisense oligomers” can also be used to describe the microRNA molecules of the present disclosure. Names of miRNAs and their sequences related to the present disclosure are provided herein. MicroRNAs recognize and bind to target mRNAs through imperfect base pairing leading to destabilization or translational inhibition of the target mRNA and thereby downregulate target gene expression. Conversely, targeting miRNAs via molecules comprising a miRNA binding site (generally a molecule comprising a sequence complementary to the seed region of the miRNA) can reduce or inhibit the miRNA-induced translational inhibition leading to an upregulation of the target gene.

The terms “mismatch” or “mismatches” refer to one or more nucleobases (whether contiguous or separate) in an oligomer nucleobase sequence (e.g., miR-485 inhibitor) that are not matched to a target nucleic acid sequence (e.g., miR-485) according to base pairing rules. While perfect complementarity is often desired, in some aspects, one or more (e.g., 6, 5, 4, 3, 2, or 1 mismatches) can occur with respect to the target nucleic acid sequence. Variations at any location within the oligomer are included. In certain aspects, antisense oligomers of the disclosure (e.g., miR-485 inhibitor) include variations in nucleobase sequence near the termini, variations in the interior, and if present are typically within about 6, 5, 4, 3, 2, or 1 subunits of the 5′ and/or 3′ terminus. In some aspects, one, two, or three nucleobases can be removed and still provide on-target binding.

As used herein, the terms “modulate,” “modify,” and grammatical variants thereof, generally refer when applied to a specific concentration, level, expression, function or behavior, to the ability to alter, by increasing or decreasing, e.g., directly or indirectly promoting/stimulating/up-regulating or interfering with/inhibiting/down-regulating the specific concentration, level, expression, function or behavior, such as, e.g., to act as an antagonist or agonist. In some instances, a modulator can increase and/or decrease a certain concentration, level, activity or function relative to a control, or relative to the average level of activity that would generally be expected or relative to a control level of activity. In some aspects, a miRNA inhibitor disclosed herein, e.g., a miR-485 inhibitor, can modulate (e.g., decrease, alter, or abolish) miR-485 expression, function, and/or activity, and thereby, modulate SIRT1 protein or gene expression and/or activity.

“Nucleic acid,” “nucleic acid molecule,” “nucleotide sequence,” “polynucleotide,” and grammatical variants thereof are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein.

The terms “pharmaceutically acceptable carrier,” “pharmaceutically acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

As used herein, the term “pharmaceutical composition” refers to one or more of the compounds described herein, such as, e.g., a miRNA inhibitor of the present disclosure, mixed or intermingled with, or suspended in one or more other chemical components, such as pharmaceutically acceptable carriers and excipients. One purpose of a pharmaceutical composition is to facilitate administration of preparations comprising a miRNA inhibitor of the present disclosure to a subject.

The term “polynucleotide,” as used herein, refers to polymers of nucleotides of any length, including ribonucleotides, deoxyribonucleotides, analogs thereof, or mixtures thereof.

In some aspects, the term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide.

In some aspects, the term “polynucleotide” includes polydeoxyribonucleotides (containing 2-deoxy-D-ribose), polyribonucleotides (containing D-ribose), including tRNA, rRNA, shRNA, siRNA, miRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA.

In some aspects of the present disclosure, a polynucleotide can be, e.g., an oligonucleotide, such as an antisense oligonucleotide. In some aspects, the oligonucleotide is an RNA. In some aspects, the RNA is a synthetic RNA. In some aspects, the synthetic RNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine).

The terms “polypeptide,” “peptide,” and “protein” are used interchangeably herein to refer to polymers of amino acids of any length, e.g., that are encoded by the SIRT1 gene. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. The term “polypeptide,” as used herein, refers to proteins, polypeptides, and peptides of any size, structure, or function.

Polypeptides include gene products, naturally occurring polypeptides, synthetic polypeptides, homologs, orthologs, paralogs, fragments and other equivalents, variants, and analogs of the foregoing.

A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to about 50 amino acids long, e.g., about 5, about 10, about 15, about 20, about 25, about 30, about 35, about 40, about 45, or about 50 amino acids long.

The terms “prevent,” “preventing,” and variants thereof as used herein, refer partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, preventing an outcome is achieved through prophylactic treatment.

As used herein, the terms “promoter” and “promoter sequence” are interchangeable and refer to a DNA sequence capable of controlling the expression of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

The promoter sequence is typically bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase. In some aspects, a promoter that can be used with the present disclosure includes a tissue specific promoter.

As used herein, “prophylactic” refers to a therapeutic or course of action used to prevent the onset of a disease or condition, or to prevent or delay a symptom associated with a disease or condition.

As used herein, a “prophylaxis” refers to a measure taken to maintain health and prevent the onset of a disease or condition, or to prevent or delay a symptom associated with a disease or condition.

As used herein, the term “gene regulatory region” or “regulatory region” refers to nucleotide sequences located upstream (5′ non-coding sequences), within, or downstream (3′ non-coding sequences) of a coding region, and which influence the transcription, RNA processing, stability, or translation of the associated coding region. Regulatory regions can include promoters, translation leader sequences, introns, polyadenylation recognition sequences, RNA processing sites, effector binding sites, or stem-loop structures. If a coding region is intended for expression in a eukaryotic cell, a polyadenylation signal and transcription termination sequence will usually be located 3′ to the coding sequence.

In some aspects, a miR-485 inhibitor disclosed herein (e.g., a polynucleotide encoding a RNA comprising one or more miR-485 binding site) can include a promoter and/or other expression (e.g., transcription) control elements operably associated with one or more coding regions. In an operable association a coding region for a gene product is associated with one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed. Other expression control elements, besides a promoter, for example enhancers, operators, repressors, and transcription termination signals, can also be operably associated with a coding region to direct gene product expression.

As used herein, the term “similarity” refers to the overall relatedness between polymeric molecules, e.g. between polynucleotide molecules (e.g. miRNA molecules). Calculation of percent similarity of polymeric molecules to one another can be performed in the same manner as a calculation of percent identity, except that calculation of percent similarity takes into account conservative substitutions as is understood in the art. It is understood that percentage of similarity is contingent on the comparison scale used, i.e., whether the nucleic acids are compared, e.g., according to their evolutionary proximity, charge, volume, flexibility, polarity, hydrophobicity, aromaticity, isoelectric point, antigenicity, or combinations thereof.

The terms “subject,” “patient,” “individual,” and “host,” and variants thereof are used interchangeably herein and refer to any mammalian subject, including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The methods described herein are applicable to both human therapy and veterinary applications.

As used herein, the phrase “subject in need thereof” includes subjects, such as mammalian subjects, that would benefit from administration of a miRNA inhibitor of the disclosure (e.g., miR-485 inhibitor), e.g., to increase the expression level of SIRT1 protein and/or SIRT1 gene.

As used herein, the term “therapeutically effective amount” is the amount of reagent or pharmaceutical compound comprising a miRNA inhibitor of the present disclosure that is sufficient to a produce a desired therapeutic effect, pharmacologic and/or physiologic effect on a subject in need thereof. A therapeutically effective amount can be a “prophylactically effective amount” as prophylaxis can be considered therapy.

The terms “treat,” “treatment,” or “treating,” as used herein refers to, e.g., the reduction in severity of a disease or condition; the reduction in the duration of a disease course; the amelioration or elimination of one or more symptoms associated with a disease or condition (e.g., Alzheimer's); the provision of beneficial effects to a subject with a disease or condition, without necessarily curing the disease or condition. The term also includes prophylaxis or prevention of a disease or condition or its symptoms thereof.

The term “upstream” refers to a nucleotide sequence that is located 5′ to a reference nucleotide sequence.

A “vector” refers to any vehicle for the cloning of and/or transfer of a nucleic acid into a host cell. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “vector” includes both viral and nonviral vehicles for introducing the nucleic acid into a cell in vitro, ex vivo or in vivo. A large number of vectors are known and used in the art including, for example, plasmids, modified eukaryotic viruses, or modified bacterial viruses. Insertion of a polynucleotide into a suitable vector can be accomplished by ligating the appropriate polynucleotide fragments into a chosen vector that has complementary cohesive termini.

Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters.

### II. Diagnostic Methods

The present disclosure provides a method of identifying a subject responsive to a miR-485 inhibitor therapy comprising measuring a level of a SIRT1 protein and/or a SIRT1 gene in the subject. The SIRT1 protein and/or SIRT1 gene can be measured using any suitable methods known in the art, including those described herein (see, e.g., Example 1). In some aspects, the subject is previously administered a compound that inhibits miR-485 expression and/or activity (miRNA inhibitor).

In some aspects, the methods further comprise administering a compound that inhibits miR-485 (miRNA inhibitor). In some aspects, the subject has a disease or a condition associated with a decreased level of a SIRT1 protein and/or a SIRT1 gene. In some aspects, the miRNA inhibitor induces autophagy and/or treats or prevents inflammation.

The present disclosure comprises a method of treating a disease or condition associated with an abnormal level of a SIRT1 protein and/or a SIRT1 gene in a subject in need thereof comprising administering to the subject a compound that inhibits miR-485 (miRNA inhibitor) and measuring a level of a SIRT1 protein and/or a SIRT1 gene in the subject. In some aspects, the level of the SIRT1 protein and/or SIRT1 gene is increased after the administration. In some aspects, the method further comprises administering a second dose of the miRNA inhibitor to the subject.

In some aspects, the level of a SIRT1 protein and/or a SIRT1 gene in the subject is increased at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% in a frontal cortex section compared to the level prior to the administration. In some aspects, the level of a SIRT1 protein and/or a SIRT1 gene is increased at least about 300% in the frontal cortex of mice after administration of a single IV dose of the miR485 inhibitor (100 μg/mouse). In some aspects, the SIRT1 protein expression is increased within about 24 hours after the administration. In some aspects, the SIRT1 protein expression is increased within about 48 hours after the administration of a miRNA inhibitor.

In some aspects, the level of a SIRT1 protein and/or a SIRT1 gene in the subject is increased at least about 30%, at least about 40%, at least about 50%, at least about 60%, at least about 70%, at least about 80%, at least about 90%, at least about 100%, at least about 110%, at least about 120%, at least about 130%, at least about 140%, or at least about 150% in a hippocampus section compared to the level prior to the administration. In some aspects, the level of a SIRT1 protein is increased in a hippocampus section of mice after administration of a single IV dose of the miR485 inhibitor (100 μg/mouse). In some aspects, the SIRT1 protein expression is increased within about 24 hours. In some aspects, the SIRT1 protein expression is increased within about 48 hours.

In some aspects, the level of a SIRT1 protein and/or a SIRT1 gene in the subject is increased at least about 30%, at least about 40%, at least about 50%, at least about 60%, at least about 70%, at least about 80%, at least about 90%, or at least about 100% in serum compared to the level prior to the administration. In some aspects, the level of a SIRT1 protein is increased at least about 100% in serum of mice after administration of a single IV dose of the miR485 inhibitor (100 μg/mouse). In some aspects, the SIRT1 protein expression is increased within about 24 hours. In some aspects, the SIRT1 protein expression is increased within about 48 hours.

As described herein, in some aspects, the present disclosure is also directed to a method of identifying a subject who is responsive to an miR485 inhibitor therapy comprising measuring a level of a SIRT1 protein and/or a SIRT1 gene in the serum of a subject prior to and/or after the miR485 inhibitor therapy. In some aspects, the present methods comprise measuring a level of a SIRT1 protein and/or a SIRT1 gene in the serum of a subject prior to and/or after the miR485 inhibitor therapy, wherein the subject is treated with the miR485 inhibitor prior to the measuring. In any of the identification methods provided herein, in some aspects, the increase in the level of a SIRT1 and/or a SIRT1 gene indicates that the subject is responsive to the miRNA inhibitor therapy. As described herein, one or more additional doses of a miRNA inhibitor (e.g., such as those described herein) can be administered to such subjects identified as being responsive to a miRNA inhibitor therapy.

### III. Methods of Treatment

SIRT1 Regulation

In some aspects, the present disclosure provides a method of increasing an expression of a SIRT1 protein and/or a SIRT1 gene in a subject in need thereof, comprising administering to the subject a compound that inhibits miR-485 activity (i.e., miR-485 inhibitor; also referred to herein as “miRNA inhibitor”). In certain aspects, inhibiting miR-485 activity increases the expression of a SIRT1 protein and/or SIRT1 gene in the subject. The methods can be further followed by the diagnostic methods disclosed above to determine the efficacy of the compound.

Sirtuin 1 (SIRT1), also known as NAD-dependent deacetylase sirtuin-1, is a protein that in humans is encoded by the SIRT1 gene. The SIRT1 gene is located on chromosome 10 in humans (nucleotides 67,884,656 to 67,918,390 of GenBank Accession Number NC_000010.11, plus strand orientation). Synonyms of the SIRT1 gene, and the encoded protein thereof, are known and include “regulatory protein SIR2 homolog 1,” “silent mating-type information regulation 2 homolog 1,” “SIR2,” “SIR2-Like Protein 1,” “SIR2L1,” “SIR2alpha,” “Sirtuin Type 1,” “hSIRT1,” or “hSIR2.”

There are at least two known isoforms of human SIRT1 protein, resulting from alternative splicing. SIRT1 isoform 1 (UniProt identifier: Q96EB6-1) consists of 747 amino acids and has been chosen as the canonical sequence (SEQ ID NO: 31). SIRT1 isoform 2 (also known as “delta-exon8) (UniProt identifier: Q96EB6-2) consists of 561 amino acids and differs from the canonical sequence as follows: 454-639: missing (SEQ ID NO: 32). Table 1 below provides the sequences for the two SIRT1 isoforms.

TABLE 1
SIRT1 Protein Isoforms

| | |
|---|---|
| Isoform 1 | MADEAALALQPGGSPSAAGADREAASSPAGEPLRKRPRRDGPGLERSPGEPGGAAPEREV |
| (UniProt: | PAAARGCPGAAAAALWREAEAEAAAAGGEQEAQATAAAGEGDNGPGLQGPSREPPLADNL |
| Q96EB6-1) | YDEDDDDEGEEEEEAAAAAIGYRDNLLFGDEIITNGFHSCESDEEDRASHASSSDWTPRP |
| (SEQ ID | RIGPYTFVQQHLMIGTDPRTILKDLLPETIPPPELDDMTLWQIVINILSEPPKRKKRKDI |
| NO: 31) | NTIEDAVKLLQECKKIIVLTGAGVSVSCGIPDFRSRDGIYARLAVDFPDLPDPQAMFDIE |
| | YFRKDPRPFFKFAKEIYPGQFQPSLCHKFIALSDKEGKLLRNYTQNIDTLEQVAGIQRII |
| | QCHGSFATASCLICKYKVDCEAVRGDIFNQVVPRCPRCPADEPLAIMKPEIVFFGENLPE |
| | QFHRAMKYDKDEVDLLIVIGSSLKVRPVALIPSSIPHEVPQILINREPLPHLHFDVELLG |
| | DCDVIINELCHRLGGEYAKLCCNPVKLSEITEKPPRTQKELAYLSELPPTPLHVSEDSSS |
| | PERTSPPDSSVIVTLLDQAAKSNDDLDVSESKGCMEEKPQEVQTSRNVESIAEQMENPDL |
| | KNVGSSTGEKNERTSVAGTVRKCWPNRVAKEQISRRLDGNQYLFLPPNRYIFHGAEVYSD |
| | SEDDVLSSSSCGSNSDSGTCQSPSLEEPMEDESEIEEFYNGLEDEPDVPERAGGAGFGTD |
| | GDDQEAINEAISVKQEVTDMNYPSNKS |
| Isoform 2 | MADEAALALQPGGSPSAAGADREAASSPAGEPLRKRPRRDGPGLERSPGEPGGAAPEREV |
| (UniProt: | PAAARGCPGAAAAALWREAEAEAAAAGGEQEAQATAAAGEGDNGPGLQGPSREPPLADNL |
| Q96EB6-2) | YDEDDDDEGEEEEEAAAAAIGYRDNLLFGDEIITNGFHSCESDEEDRASHASSSDWTPRP |
| (SEQ ID | RIGPYTFVQQHLMIGTDPRTILKDLLPETIPPPELDDMTLWQIVINILSEPPKRKKRKDI |
| NO: 32) | NTIEDAVKLLQECKKIIVLTGAGVSVSCGIPDFRSRDGIYARLAVDFPDLPDPQAMFDIE |
| | YFRKDPRPFFKFAKEIYPGQFQPSLCHKFIALSDKEGKLLRNYTQNIDTLEQVAGIQRII |
| | QCHGSFATASCLICKYKVDCEAVRGDIFNQVVPRCPRCPADEPLAIMKPEIVFFGENLPE |
| | QFHRAMKYDKDEVDLLIVIGSSLKVRPVALIPSNQYLFLPPNRYIFHGAEVYSDSEDDVL |
| | SSSSCGSNSDSGTCQSPSLEEPMEDESEIEEFYNGLEDEPDVPERAGGAGFGTDGDDQEA |
| | INEAISVKQEVTDMNYPSNKS |

As used herein, the term “SIRT1” includes any variants or isoforms of SIRT1 which are naturally expressed by cells. Accordingly, in some aspects, a miR-485 inhibitor disclosed herein can increase the expression of SIRT1 isoform 1. In some aspects, a miR-485 inhibitor disclosed herein can increase the expression of SIRT1 isoform 2. In further aspects, a miR-485 inhibitor disclosed herein can increase the expression of both SIRT1 isoform 1 and isoform 2. Unless indicated otherwise, both isoform 1 and isoform 2 are collectively referred to herein as “SIRT1.”

In some aspects, a miR-485 inhibitor of the present disclosure increases the expression of SIRT1 protein and/or SIRT1 gene by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300% compared to a reference (e.g., expression of SIRT1 protein and/or SIRT1 gene in a corresponding subject that did not receive an administration of the miR-485 inhibitor).

Not to be bound by any one theory, in some aspects, a miR-485 inhibitor disclosed herein increases the expression of SIRT1 protein and/or SIRT1 gene by reducing the expression and/or activity of miR-485, e.g., miR-485-3p. In some aspects, a miR-485 inhibitor of the present disclosure can reduce the expression and/or activity of miR-485-3p.

In some aspects, a miR-485 inhibitor disclosed herein decreases the expression and/or activity of miR-485-3p by at least about 5%, at least about 10%, at least about 20%, at least about 30%, at least about 40%, at least about 50%, at least about 60%, at least about 70%, at least about 80%, at least about 90%, or about 100% compared to a reference (e.g., miR-485-3p expression in a corresponding subject that did not receive an administration of the miR-485 inhibitor). In certain aspects, a miR-485 inhibitor disclosed herein decreases the expression and/or activity of miR-485-5p by at least about 5%, at least about 10%, at least about 20%, at least about 30%, at least about 40%, at least about 50%, at least about 60%, at least about 70%, at least about 80%, at least about 90%, or about 100% compared to a reference (e.g., miR-485-5p expression in a corresponding subject that did not receive an administration of the miR-485 inhibitor). In further aspects, a miR-485 inhibitor disclosed herein decreases the expression and/or activity of both miR-485-3p and miR-485-5p by at least about 5%, at least about 10%, at least about 20%, at least about 30%, at least about 40%, at least about 50%, at least about 60%, at least about 70%, at least about 80%, at least about 90%, or about 100% compared to a reference (e.g., miR-485-3p and miR-485-5p expression in a corresponding subject that did not receive an administration of the miR-485 inhibitor). In some aspects, the expression of miR-485-3p and/or miR-485-5p is completely inhibited after the administration of the miR-485 inhibitor.

As described herein, a miR-485 inhibitor of the present disclosure can increase the expression of SIRT1 protein and/or SIRT1 gene when administered to a subject. Accordingly, in some aspects, the present disclosure provides a method of treating a disease or condition associated with an abnormal (e.g., reduced) level of a SIRT1 protein and/or SIRT1 gene in a subject in need thereof. In certain aspects, the method comprises administering to the subject a compound that inhibits miR-485 activity (i.e., miR-485 inhibitor), wherein the miR-485 inhibitor increases the level of the SIRT1 protein and/or SIRT1 gene.

CD36 Regulation

As described herein, Applicant has identified that the human CD36 3′-UTR comprises a target site for miR-485-3p and that the binding of miR-485-3p can decrease CD36 expression (see, e.g., Examples 7 and 8). Accordingly, in some aspects, the present disclosure provides a method of increasing an expression of a CD36 protein and/or a CD36 gene in a subject in need thereof, comprising administering to the subject a compound that inhibits miR-485 activity (i.e., miR-485 inhibitor). In certain aspects, inhibiting miR-485 activity increases the expression of a CD36 protein and/or CD36 gene in the subject.

Cluster determinant 36 (CD36) is also known as platelet glycoprotein 4, is a protein that in humans is encoded by the CD36 gene. The CD36 gene is located on chromosome 7 (nucleotides 80,602,656 to 80,679,277 of GenBank Accession Number NC_000007.14, plus strand orientation). Synonyms of the CD36 gene, and the encoded protein thereof, are known and include “platelet glycoprotein IV,” “fatty acid translocase,” “scavenger receptor class B member 3,” “glycoprotein 88,” “glycoprotein IIIb,” “glycoprotein IV,” “thrombospondin receptor,” “GPIIIB,” “PAS IV,” “GP3B,” “GPIV,” “FAT,” “GP4,” “BDPLT10,” “SCARB3,” “CHDS7,” “PASIV,” or “PAS-4.”

There are at least four known isoform of human CD36 protein, resulting from alternative splicing. CD36 isoform 1 (UniProtidentifier: P16671-1) consists of 472 amino acids and has been chosen as the canonical sequence (SEQ ID NO: 36). CD36 isoform 2 (also known as “ex8-del”) (UniProt identifier: P16671-2) consists of 288 amino acids and differs from the canonical sequence as follows: 274-288: SIYAVFESDVNLKGI→ETCVHFTSSFSVCKS; and 289-472: missing (SEQ ID NO: 37). CD36 Isoform 3 (also known as “ex6-7-del”) (UniProt identifier: P16671-3) consists of 433 amino acids and differs from the canonical sequence as follows: 234-272: missing (SEQ ID NO: 38). CD36 isoform 4 (also known as “ex4-del” (UniProt identifier: P16671-4) consists of 412 amino acids and differs from the canonical sequence as follows: 144-203: missing (SEQ ID NO: 39). Table 2 below provides the sequences for the four CD36 isoforms.

TABLE 2
CD36 Protein Isoforms

| | |
|---|---|
| Isoform 1 | MGCDRNCGLIAGAVIGAVLAVFGGILMPVGDLLIQKTIKKQVVLEEGTIAFKNWVKTGTE |
| (UniProt: | VYRQFWIFDVQNPQEVMMNSSNIQVKQRGPYTYRVRFLAKENVTQDAEDNTVSFLQPNGA |
| P16671-1) | IFEPSLSVGTEADNFTVLNLAVAAASHIYQNQFVQMILNSLINKSKSSMFQVRTLRELLW |
| (SEQ ID NO: | GYRDPFLSLVPYPVTTTVGLFYPYNNTADGVYKVFNGKDNISKVAIIDTYKGKRNLSYWE |
| 36) | SHCDMINGTDAASFPPFVEKSQVLQFFSSDICRSIYAVFESDVNLKGIPVYRFVLPSKAF |
| | ASPVENPDNYCFCTEKIISKNCTSYGVLDISKCKEGRPVYISLPHFLYASPDVSEPIDGL |
| | NPNEEEHRTYLDIEPITGFTLQFAKRLQVNLLVKPSEKIQVLKNLKRNYIVPILWLNETG |
| | TIGDEKANMFRSQVTGKINLLGLIEMILLSVGVVMFVAFMISYCACRSKTIK |
| Isoform 2 | MGCDRNCGLIAGAVIGAVLAVFGGILMPVGDLLIQKTIKKQVVLEEGTIAFKNWVKTGTE |
| (UniProt: | VYRQFWIFDVQNPQEVMMNSSNIQVKQRGPYTYRVRFLAKENVTQDAEDNTVSFLQPNGA |
| P16671-2) | IFEPSLSVGTEADNFTVLNLAVAAASHIYQNQFVQMILNSLINKSKSSMFQVRTLRELLW |
| (SEQ ID NO: 37) | GYRDPFLSLVPYPVTTTVGLFYPYNNTADGVYKVFNGKDNISKVAIIDTYKGKRNLSYWE |
| | SHCDMINGTDAASFPPFVEKSQVLQFFSSDICRETCVHFTSSFSVCKS |
| Isoform 3 | MGCDRNCGLIAGAVIGAVLAVFGGILMPVGDLLIQKTIKKQVVLEEGTIAFKNWVKTGTE |
| (UniProt: | VYRQFWIFDVQNPQEVMMNSSNIQVKQRGPYTYRVRFLAKENVTQDAEDNTVSFLQPNGA |
| P16671-3) (SEQ | IFEPSLSVGTEADNFTVLNLAVAAASHIYQNQFVQMILNSLINKSKSSMFQVRTLRELLW |
| ID NO: 38) | GYRDPFLSLVPYPVTTTVGLFYPYNNTADGVYKVFNGKDNISKVAIIDTYKGKRSIYAVF |
| | ESDVNLKGIPVYRFVLPSKAFASPVENPDNYCFCTEKIISKNCTSYGVLDISKCKEGRPV |
| | YISLPHFLYASPDVSEPIDGLNPNEEEHRTYLDIEPITGFTLQFAKRLQVNLLVKPSEKI |
| | QVLKNLKRNYIVPILWLNETGTIGDEKANMFRSQVTGKINLLGLIEMILLSVGVVMFVAF |
| | MISYCACRSKTIK |
| Isoform 4 | MGCDRNCGLIAGAVIGAVLAVFGGILMPVGDLLIQKTIKKQVVLEEGTIAFKNWVKTGTE |
| (UniProt: | VYRQFWIFDVQNPQEVMMNSSNIQVKQRGPYTYRVRFLAKENVTQDAEDNTVSFLQPNGA |
| P16671-4) (SEQ | IFEPSLSVGTEADNFTVLNLAVAYNNTADGVYKVFNGKDNISKVAIIDTYKGKRNLSYWE |
| ID NO: 39) | SHCDMINGTDAASFPPFVEKSQVLQFFSSDICRSIYAVFESDVNLKGIPVYRFVLPSKAF |
| | ASPVENPDNYCFCTEKIISKNCTSYGVLDISKCKEGRPVYISLPHFLYASPDVSEPIDGL |
| | NPNEEEHRTYLDIEPITGFTLQFAKRLQVNLLVKPSEKIQVLKNLKRNYIVPILWLNETG |
| | TIGDEKANMFRSQVTGKINLLGLIEMILLSVGVVMFVAFMISYCACRSKTIK |

As used herein, the term “CD36” includes any variants or isoforms of CD36 which are naturally expressed by cells. Accordingly, in some aspects, a miR-485 inhibitor disclosed herein can increase the expression of CD36 isoform 1. In some aspects, a miR-485 inhibitor disclosed herein can increase the expression of CD36 isoform 2. In some aspect, a miR-485 inhibitor disclosed herein can increase the expression of CD36 isoform 3. In some aspects, a miR-485 inhibitor disclosed herein can increase the expression of CD36 isoform 4. In further aspects, a miR-485 inhibitor disclosed herein can increase the expression of both CD36 isoform 1 and isoform 2, and/or isoform 3 and isoform 4, and/or isoform 1 and isoform 4, and/or isoform 2 and isoform 3. In some aspects, a miR-485 inhibitor disclosed herein can increase the expression of all CD36 isoforms. Unless indicated otherwise, isoform 1, isoform 2, isoform 3, and isoform 4 are collectively referred to herein as “CD36.”

In some aspects, a miR-485 inhibitor of the present disclosure increases the expression of CD36 protein and/or CD36 gene by at least about 500, at least about 10%, at least about 20%, at least about 30%, at least about 40%, at least about 50%, at least about 60%, at least about 70%, at least about 80%, at least about 90%, at least about 100%, at least about 150&, at least about 200%, or at least about 300% compared to a reference (e.g., expression of CD36 protein and/or CD36 gene in a corresponding subject that did not receive an administration of the miR-485 inhibitor).

Not to be bound by any one theory, in some aspects, a miR-485 inhibitor disclosed herein increases the expression of CD36 protein and/or CD36 gene by reducing the expression and/or activity of miR-485. There are two known mature forms of miR-485: miR-485-3p and miR-485-5p. As disclosed herein, in some aspects, a miR-485 inhibitor of the present disclosure can reduce the expression and/or activity of miR-485-3p. In some aspects, a miR-485 inhibitor can reduce the expression and/or activity of miR-485-5p. In further aspects, a miR-485 inhibitor disclosed herein can reduce the expression and/or activity of both miR-485-3p and miR-485-5p.

PGC1 Regulation

The disclosures provided herein demonstrates that the miR-485 inhibitors of the present disclosure can further regulate the expression of PGC-1α, e.g., in a subject suffering from a disease or disorder disclosed herein (see, e.g., Example 5). Therefore, in some aspects, the present disclosure provides a method of increasing an expression of a PGC-1α protein and/or a PGC-1α gene in a subject in need thereof, comprising administering to the subject a compound that inhibits miR-485 activity (i.e., miR-485 inhibitor). In certain aspects, inhibiting miR-485 activity increases the expression of a PGC-1α protein and/or PGC-1α gene in the subject.

Peroxisome proliferator-activated receptor gamma coactivator 1-alpha (PGC1-α), also known as PPARG Coactivator 1 Alpha or Ligand Effect Modulator-6, is a protein that in humans is encoded by the PPARGC1A gene. The PGC1-α gene is located on chromosome 4 in humans (nucleotides 23,792,021 to 24,472,905 of GenBank Accession Number NC_000004.12, plus strand orientation). Synonyms of the PGC1-α gene, and the encoded protein thereof, are known and include “PPARGC1A,” “LEM6,” “PGC1,” “PGC1A,” “PGC-lv,” “PPARGC1, “PGC1alpha,” or “PGC-1(alpha).”

There are at least nine known isoforms of human PGC1-α protein, resulting from alternative splicing. PGC1-α isoform 1 (UniProt identifier: Q9UBK2-1) consists of 798 amino acids and has been chosen as the canonical sequence (SEQ ID NO: 40). PGC1-α isoform 2 (also known as “Isoform NT-7a”) (UniProt identifier: Q9UBK2-2) consists of 271 amino acids and differs from the canonical sequence as follows: 269-271: DPK→LFL; 272-798: Missing (SEQ ID NO: 41). PGC1-α isoform 3 (also known as “Isoform B5”) (UniProt identifier: Q9UBK2-3) consists of 803 amino acids and differs from the canonical sequence as follows: 1-18: MAWDMCNQDSESVWSDIE→MDETSPRLEEDWKKVLQREAGWQ (SEQ ID NO: 42). PGC1-α isoform 4 (also known as “Isoform B4”) (UniProt identifier: Q9UBK2-4) consists of 786 amino acids and differs from the canonical sequence as follows: 1-18: MAWDMCNQDSESVWSDIE→MDEGYF (SEQ ID NO: 43). PGC1-α isoform 5 (also known as “Isoform B4-8a”) (UniProt identifier: Q9UBK2-5) consists of 289 amino acids and differs from the canonical sequence as follows: 1-18: MAWDMCNQDSESVWSDIE→MDEGYF; 294-301: LTPPTTPP→VKTNLISK; 302-798: Missing (SEQ ID NO: 44). PGC1-α isoform 6 (also known as “Isoform B5-NT”) (UniProt identifier: Q9UBK2-6) consists of 276 amino acids and differs from the canonical sequence as follows: 1-18: MAWDMCNQDSESVWSDIE→MDETSPRLEEDWKKVLQREAGWQ; 269-271: DPK→LFL; 272-798: Missing (SEQ ID NO: 45). PGC1-α isoform 7 (also known as “B4-3ext”) (UniProt identifier: Q9UBK2-7) consists of 138 amino acids and differs from the canonical sequence as follows: 1-18: MAWDMCNQDSESVWSDIE→MDEGYF; 144-150: LKKLLLA→VRTLPTV; 151-798: Missing (SEQ ID NO: 46). PGC1-α isoform 8 (also known as “Isoform 8a”) (UniProt identifier: Q9UBK2-8) consists of 301 amino acids and differs from the canonical sequence as follows: 294-301: LTPPTTPP→VKTNLISK; 302-798: Missing (SEQ ID NO: 47). PGC1-α isoform 9 (also known as “Isoform 9” or “L-PGG-1alpha”) (UniProt identifier: Q9UBK2-9) consists of 671 amino acids and differs from the canonical sequence as follows: 1-127: Missing (SEQ ID NO: 48). Table 3 below provides the sequences for the nine PGC1-α isoforms.

TABLE 3
PGC1-α Protein Isoforms

| | |
|---|---|
| Isoform 1 | MAWDMCNQDSESVWSDIECAALVGEDQPLCPDLPELDLSELDVNDLDTDSFLGGLKWCSD |
| (UniProt: | QSEIISNQYNNEPSNIFEKIDEENEANLLAVLTETLDSLPVDEDGLPSFDALTDGDVTTD |
| Q9UBK2-1) | NEASPSSMPDGTPPPQEAEEPSLLKKLLLAPANTQLSYNECSGLSTQNHANHNHRIRTNP |
| (SEQ ID | AIVKTENSWSNKAKSICQQQKPQRRPCSELLKYLTTNDDPPHTKPTENRNSSRDKCTSKK |
| NO: 40) | KSHTQSQSQHLQAKPTTLSLPLTPESPNDPKGSPFENKTIERTLSVELSGTAGLTPPTTP |
| | PHKANQDNPFRASPKLKSSCKTVVPPPSKKPRYSESSGTQGNNSTKKGPEQSELYAQLSK |
| | SSVLTGGHEERKTKRPSLRLFGDHDYCQSINSKTEILINISQELQDSRQLENKDVSSDWQ |
| | GQICSSTDSDQCYLRETLEASKQVSPCSTRKQLQDQEIRAELNKHFGHPSQAVFDDEADK |
| | TGELRDSDFSNEQFSKLPMFINSGLAMDGLFDDSEDESDKLSYPWDGTQSYSLFNVSPSC |
| | SSFNSPCRDSVSPPKSLFSQRPQRMRSRSRSFSRHRSCSRSPYSRSRSRSPGSRSSSRSC |
| | YYYESSHYRHRTHRNSPLYVRSRSRSPYSRRPRYDSYEEYQHERLKREEYRREYEKRESE |
| | RAKQRERQRQKAIEERRVIYVGKIRPDTTRTELRDRFEVFGEIEECTVNLRDDGDSYGFI |
| | TYRYTCDAFAALENGYTLRRSNETDFELYFCGRKQFFKSNYADLDSNSDDFDPASTKSKY |
| | DSLDFDSLLKEAQRSLRR |
| Isoform 2 | MAWDMCNQDSESVWSDIECAALVGEDQPLCPDLPELDLSELDVNDLDTDSFLGGLKWCSD |
| (UniProt: | QSEIISNQYNNEPSNIFEKIDEENEANLLAVLTETLDSLPVDEDGLPSFDALTDGDVTTD |
| Q9UBK2-2) | NEASPSSMPDGTPPPQEAEEPSLLKKLLLAPANTQLSYNECSGLSTQNHANHNHRIRTNP |
| (SEQ ID | AIVKTENSWSNKAKSICQQQKPQRRPCSELLKYLTTNDDPPHTKPTENRNSSRDKCTSKK |
| NO: 41) | KSHTQSQSQHLQAKPTTLSLPLTPESPNLFL |
| Isoform 3 | MDETSPRLEEDWKKVLQREAGWQCAALVGEDQPLCPDLPELDLSELDVNDLDTDSFLGGL |
| (UniProt: | KWCSDQSEIISNQYNNEPSNIFEKIDEENEANLLAVLTETLDSLPVDEDGLPSFDALTDG |
| Q9UBK2-3) | DVTTDNEASPSSMPDGTPPPQEAEEPSLLKKLLLAPANTQLSYNECSGLSTQNHANHNHR |
| (SEQ ID | IRTNPAIVKTENSWSNKAKSICQQQKPQRRPCSELLKYLTTNDDPPHTKPTENRNSSRDK |
| NO: 42) | CTSKKKSHTQSQSQHLQAKPTTLSLPLTPESPNDPKGSPFENKTIERTLSVELSGTAGLT |
| | PPTTPPHKANQDNPFRASPKLKSSCKTVVPPPSKKPRYSESSGTQGNNSTKKGPEQSELY |
| | AQLSKSSVLTGGHEERKTKRPSLRLFGDHDYCQSINSKTEILINISQELQDSRQLENKDV |
| | SSDWQGQICSSTDSDQCYLRETLEASKQVSPCSTRKQLQDQEIRAELNKHFGHPSQAVFD |
| | DEADKTGELRDSDFSNEQFSKLPMFINSGLAMDGLFDDSEDESDKLSYPWDGTQSYSLFN |
| | VSPSCSSFNSPCRDSVSPPKSLFSQRPQRMRSRSRSFSRHRSCSRSPYSRSRSRSPGSRS |
| | SSRSCYYYESSHYRHRTHRNSPLYVRSRSRSPYSRRPRYDSYEEYQHERLKREEYRREYE |
| | KRESERAKQRERQRQKAIEERRVIYVGKIRPDTTRTELRDRFEVFGEIEECTVNLRDDGD |
| | SYGFITYRYTCDAFAALENGYTLRRSNETDFELYFCGRKQFFKSNYADLDSNSDDFDPAS |
| | TKSKYDSLDFDSLLKEAQRSLRR |
| Isoform 4 | MDEGYFCAALVGEDQPLCPDLPELDLSELDVNDLDTDSFLGGLKWCSDQSEIISNQYNNE |
| (UniProt: | PSNIFEKIDEENEANLLAVLTETLDSLPVDEDGLPSFDALTDGDVTTDNEASPSSMPDGT |
| Q9UBK2-4) | PPPQEAEEPSLLKKLLLAPANTQLSYNECSGLSTQNHANHNHRIRTNPAIVKTENSWSNK |
| (SEQ ID | AKSICQQQKPQRRPCSELLKYLTTNDDPPHTKPTENRNSSRDKCTSKKKSHTQSQSQHLQ |
| NO: 43) | AKPTTLSLPLTPESPNDPKGSPFENKTIERTLSVELSGTAGLTPPTTPPHKANQDNPFRA |
| | SPKLKSSCKTWPPPSKKPRYSESSGTQGNNSTKKGPEQSELYAQLSKSSVLTGGHEERK |
| | TKRPSLRLFGDHDYCQSINSKTEILINISQELQDSRQLENKDVSSDWQGQICSSTDSDQC |
| | YLRETLEASKQVSPCSTRKQLQDQEIRAELNKHFGHPSQAVFDDEADKTGELRDSDFSNE |
| | QFSKLPMFINSGLAMDGLFDDSEDESDKLSYPWDGTQSYSLFNVSPSCSSFNSPCRDSVS |
| | PPKSLFSQRPQRMRSRSRSFSRHRSCSRSPYSRSRSRSPGSRSSSRSCYYYESSHYRHRT |
| | HRNSPLYVRSRSRSPYSRRPRYDSYEEYQHERLKREEYRREYEKRESERAKQRERQRQKA |
| | IEERRVIYVGKIRPDTTRTELRDRFEVFGEIEECTVNLRDDGDSYGFITYRYTCDAFAAL |
| | ENGYTLRRSNETDFELYFCGRKQFFKSNYADLDSNSDDFDPASTKSKYDSLDFDSLLKEA |
| | QRSLRR |
| Isoform 5 | MDEGYFCAALVGEDQPLCPDLPELDLSELDVNDLDTDSFLGGLKWCSDQSEIISNQYNNE |
| (UniProt: | PSNIFEKIDEENEANLLAVLTETLDSLPVDEDGLPSFDALTDGDVTTDNEASPSSMPDGT |
| Q9UBK2-5) | PPPQEAEEPSLLKKLLLAPANTQLSYNECSGLSTQNHANHNHRIRTNPAIVKTENSWSNK |
| (SEQ ID | AKSICQQQKPQRRPCSELLKYLTTNDDPPHTKPTENRNSSRDKCTSKKKSHTQSQSQHLQ |
| NO: 44) | AKPTTLSLPLTPESPNDPKGSPFENKTIERTLSVELSGTAGVKTNLISK |
| Isoform 6 | MDETSPRLEEDWKKVLQREAGWQCAALVGEDQPLCPDLPELDLSELDVNDLDTDSFLGGL |
| (UniProt: | KWCSDQSEIISNQYNNEPSNIFEKIDEENEANLLAVLTETLDSLPVDEDGLPSFDALTDG |
| Q9UBK2-6) | DVTTDNEASPSSMPDGTPPPQEAEEPSLLKKLLLAPANTQLSYNECSGLSTQNHANHNHR |
| (SEQ ID | IRTNPAIVKTENSWSNKAKSICQQQKPQRRPCSELLKYLTTNDDPPHTKPTENRNSSRDK |
| NO: 45) | CTSKKKSHTQSQSQHLQAKPTTLSLPLTPESPNLFL |
| Isoform 7 | MDEGYFCAALVGEDQPLCPDLPELDLSELDVNDLDTDSFLGGLKWCSDQSEIISNQYNNE |
| (UniProt: | PSNIFEKIDEENEANLLAVLTETLDSLPVDEDGLPSFDALTDGDVTTDNEASPSSMPDGT |
| Q9UBK2-7) | PPPQEAEEPSLVRTLPTV |
| (SEQ ID | |
| NO: 46) | |
| Isoform 8 | MAWDMCNQDSESVWSDIECAALVGEDQPLCPDLPELDLSELDVNDLDTDSFLGGLKWCSD |
| (UniProt: | QSEIISNQYNNEPSNIFEKIDEENEANLLAVLTETLDSLPVDEDGLPSFDALTDGDVTTD |
| Q9UBK2-8) | NEASPSSMPDGTPPPQEAEEPSLLKKLLLAPANTQLSYNECSGLSTQNHANHNHRIRTNP |
| (SEQ ID | AIVKTENSWSNKAKSICQQQKPQRRPCSELLKYLTTNDDPPHTKPTENRNSSRDKCTSKK |
| NO: 47) | KSHTQSQSQHLQAKPTTLSLPLTPESPNDPKGSPFENKTIERTLSVELSGTAGVKTNLIS |
| | K |
| Isoform 9 | MPDGTPPPQEAEEPSLLKKLLLAPANTQLSYNECSGLSTQNHANHNHRIRTNPAIVKTEN |
| (UniProt: | SWSNKAKSICQQQKPQRRPCSELLKYLTTNDDPPHTKPTENRNSSRDKCTSKKKSHTQSQ |
| Q9UBK2-9) | SQHLQAKPTTLSLPLTPESPNDPKGSPFENKTIERTLSVELSGTAGLTPPTTPPHKANQD |
| (SEQ ID | NPFRASPKLKSSCKTVVPPPSKKPRYSESSGTQGNNSTKKGPEQSELYAQLSKSSVLTGG |
| NO: 48) | HEERKTKRPSLRLFGDHDYCQSINSKTEILINISQELQDSRQLENKDVSSDWQGQICSST |
| | DSDQCYLRETLEASKQVSPCSTRKQLQDQEIRAELNKHFGHPSQAVFDDEADKTGELRDS |
| | DFSNEQFSKLPMFINSGLAMDGLFDDSEDESDKLSYPWDGTQSYSLFNVSPSCSSFNSPC |
| | RDSVSPPKSLFSQRPQRMRSRSRSFSRHRSCSRSPYSRSRSRSPGSRSSSRSCYYYESSH |
| | YRHRTHRNSPLYVRSRSRSPYSRRPRYDSYEEYQHERLKREEYRREYEKRESERAKQRER |
| | QRQKAIEERRVIYVGKIRPDTTRTELRDRFEVFGEIEECTVNLRDDGDSYGFITYRYTCD |
| | AFAALENGYTLRRSNETDFELYFCGRKQFFKSNYADLDSNSDDFDPASTKSKYDSLDFDS |
| | LLKEAQRSLRR |

As used herein, the term “PGC1-α” includes any variants or isoforms of PGC1-α which are naturally expressed by cells. Accordingly, in some aspects, a miR-485 inhibitor disclosed herein can increase the expression of PGC1-α isoform 1. In some aspects, a miR-485 inhibitor disclosed herein can increase the expression of PGC1-α isoform 2. Accordingly, in some aspects, a miR-485 inhibitor disclosed herein can increase the expression of PGC1-α isoform 1. In some aspects, a miR-485 inhibitor disclosed herein can increase the expression of PGC1-α isoform 2. Accordingly, in some aspects, a miR-485 inhibitor disclosed herein can increase the expression of PGC1-α isoform 3. In some aspects, a miR-485 inhibitor disclosed herein can increase the expression of PGC1-α isoform 4. Accordingly, in some aspects, a miR-485 inhibitor disclosed herein can increase the expression of PGC1-α isoform 5. In some aspects, a miR-485 inhibitor disclosed herein can increase the expression of PGC1-α isoform 6. Accordingly, in some aspects, a miR-485 inhibitor disclosed herein can increase the expression of PGC1-α isoform 7. In some aspects, a miR-485 inhibitor disclosed herein can increase the expression of PGC1-α isoform 8. Accordingly, in some aspects, a miR-485 inhibitor disclosed herein can increase the expression of PGC1-α isoform 9. In further aspects, a miR-485 inhibitor disclosed herein can increase the expression of PGC1-α isoform 1, isoform 2, isoform 3, isoform 4, isoform 5, isoform 6, isoform 7, isoform 8, and isoform 9. Unless indicated otherwise, both isoform 1 and isoform 2 are collectively referred to herein as “PGC1-α.”

In some aspects, a miR-485 inhibitor of the present disclosure increases the expression of PGC1-α protein and/or PGC1-α gene by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300% compared to a reference (e.g., expression of PGC1-α protein and/or PGC1-α gene in a corresponding subject that did not receive an administration of the miR-485 inhibitor).

Not to be bound by any one theory, in some aspects, a miR-485 inhibitor disclosed herein increases the expression of PGC1-α protein and/or PGC1-α gene by reducing the expression and/or activity of miR-485. There are two known mature forms of miR-485: miR-485-3p and miR-485-5p. In some aspects, a miR-485 inhibitor of the present disclosure can reduce the expression and/or activity of miR-485-3p. In some aspects, a miR-485 inhibitor can reduce the expression and/or activity of miR-485-5p. In further aspects, a miR-485 inhibitor disclosed herein can reduce the expression and/or activity of both miR-485-3p and miR-485-5p.

As will be apparent from the present disclosure, any disease or condition associated with abnormal (e.g., reduced) level of a SIRT1 protein and/or SIRT1 gene can be treated with the present disclosure. In some aspects, the present disclosure can be useful in treating any disease or condition associated with abnormal (e.g., reduced) level of a CD36 protein and/or CD36 gene. In some aspects, the present disclosure can also be used to treat a disease or disorder associated with abnormal (e.g., reduced) level of a PGC1-α protein and/or PGC1-α gene. In some aspects, a disease or condition associated with abnormal (e.g., reduced) level of such proteins and/or genes comprises a neurodegenerative disease or disorder. As used herein, the term “neurodegenerative disease or disorder” refers to a disease or disorder caused by the progressive pathologic changes within the nervous system, particularly within the neurons of the brain. In some aspects, such progressive destruction of the nervous system can result in physical (e.g., ataxias) and/or mental (e.g., dementia) impairments. Non-limiting examples of neurodegenerative diseases or disorders that can be treated with the present disclosure include Alzheimer's disease, Parkinson's disease, or any combination thereof. Other diseases or conditions that can be treated with the present disclosure include, but are not limited to, autism spectrum disorder, mental retardation, seizure, stroke, spinal cord injury, or any combination thereof.

In some aspects, a disease or disorder that can be treated with the present disclosure comprises Alzheimer's disease. In certain aspects, Alzheimer's disease comprises pre-dementia Alzheimer's disease, early Alzheimer's disease, moderate Alzheimer's disease, advanced Alzheimer's disease, early onset familial Alzheimer's disease, inflammatory Alzheimer's disease, non-inflammatory Alzheimer's disease, cortical Alzheimer's disease, early-onset Alzheimer's disease, late-onset Alzheimer's disease, or any combination thereof.

In some aspects, administering a miR-485 inhibitor disclosed herein can improve one or more symptoms of a disease or condition associated with abnormal (e.g., reduced) levels of SIRT1 protein and/or SIRT1 gene. In some aspects, administering a miR-485 inhibitor disclosed herein can improve one or more symptoms of a disease or condition associated with abnormal (e.g., reduced) levels of CD36 protein and/or CD36 gene. In some aspects, administering a miR-485 inhibitor disclosed herein can improve one or more symptoms of a disease or condition associated with abnormal (e.g., reduced) levels of PGC1-α protein and/or PGC1-α gene. Non-limiting examples of such symptoms are described below.

In some aspects, administering a miR-485 inhibitor of the present disclosure reduces the occurrence or risk of occurrence of one or more symptoms of cognitive impairments in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a reference (e.g., subjects that did not receive an administration of the miR-485 inhibitor).

In some aspects, administering a miR-485 inhibitor of the present disclosure reduces memory loss in a subject (e.g., suffering from a neurodegenerative disease) compared to a reference (e.g., memory loss in the subject prior to the administering). In some aspects, administering a miR-485 inhibitor of the present disclosure reduces memory loss or the risk of occurrence of memory loss in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a reference (e.g., subjects that did not receive an administration of the miR-485 inhibitor).

In some aspects, administering a miR-485 inhibitor of the present disclosure improves memory retention in a subject (e.g., suffering from a neurodegenerative disease) compared to a reference (e.g., memory retention in the subject prior to the administering). In some aspects, administering a miR-485 inhibitor of the present disclosure improves and/or increases memory retention in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 150%, at least about 200%, at least about 250%, or at least about 300% or more compared to a reference (e.g., subjects that did not receive an administration of the miR-485 inhibitor).

In some aspects, administering a miR-485 inhibitor of the present disclosure improves spatial working memory in a subject (e.g., suffering from a neurodegenerative disease) compared to a reference (e.g., spatial working memory in the subject prior to the administering). As used herein, the term “spatial working memory” refers to the ability to keep spatial information activity in working memory over a short period of time. In some aspects, spatial working memory is improved and/or increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, or at least about 300% or more compared to a reference (e.g., subjects that did not receive an administration of the miR-485 inhibitor).

In some aspects, administering a miR-485 inhibitor of the present disclosure increases the phagocytic activity of scavenger cells (e.g., glial cells) (e.g., by increasing the expression of CD36 protein and/or CD36 gene) in a subject (e.g., suffering from a neurodegenerative disease) compared to a reference (e.g., phagocytic activity in the subject prior to the administering). In some aspects, administering a miR-485 inhibitor of the present disclosure increases dendritic spine density of a neuron in a subject (e.g., suffering from a neurodegenerative disease) by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, or at least about 300% or more compared to a reference (e.g., subjects that did not receive an administration of the miR-485 inhibitor).

In some aspects, administering a miR-485 inhibitor of the present disclosure reduces an amyloid beta (Aβ) plaque load in a subject (e.g., suffering from a neurodegenerative disease) (e.g., by increasing the expression of CD36 protein and/or CD36 gene) compared to a reference (e.g., amyloid beta (Aβ) plaque load in the subject prior to the administering). As used herein, “amyloid beta plaque” refers to all forms of aberrant deposition of amyloid beta including large aggregates and small associations of a few amyloid beta peptides and can contain any variation of the amyloid beta peptides. Amyloid beta (Aβ) plaque is known to cause neuronal changes, e.g., aberrations in synapse composition, synapse shape, synapse density, loss of synaptic conductivity, changes in dendrite diameter, changes in dendrite length, changes in spine density, changes in spine area, changes in spine length, or changes in spine head diameter. In some aspects, administering a miR-485 inhibitor of the present disclosure reduces an amyloid beta plaque load in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 85%, at least about 90%, at least about 95%, or about 100% compared to a reference (e.g., subjects that did not receive an administration of the miR-485 inhibitor).

In some aspects, administering a miR-485 inhibitor disclosed herein increases neurogenesis in a subject (e.g., suffering from a neurodegenerative disease) (e.g., by increasing the expression of CD36 protein and/or CD36 gene) compared to a reference (e.g., neurogenesis in the subject prior to the administering). As used herein, the term “neurogenesis” refers to the process by which neurons are created. Neurogenesis encompasses proliferation of neural stem and progenitor cells, differentiation of these cells into new neural cell types, as well as migration and survival of the new cells. The term is intended to cover neurogenesis as it occurs during normal development, predominantly during pre-natal and peri-natal development, as well as neural cells regeneration that occurs following disease, damage or therapeutic intervention. Adult neurogenesis is also termed “nerve” or “neural” regeneration. In some aspects, administering a miR-485 inhibitor of the present disclosure increases neurogenesis in a subject (e.g., suffering from a neurodegenerative disease) by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, or at least about 300% or more compared to a reference (e.g., subjects that did not receive an administration of the miR-485 inhibitor).

In some aspects, increasing and/or inducing neurogenesis is associated with increased proliferation, differentiation, migration, and/or survival of neural stem cells and/or progenitor cells. Accordingly, in some aspects, administering a miR-485 inhibitor of the present disclosure can increase the proliferation of neural stem cells and/or progenitor cells in the subject. In certain aspects, the proliferation of neural stem cells and/or progenitor cells is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, or at least about 300% or more compared to a reference (e.g., subjects that did not receive an administration of the miR-485 inhibitor). In some aspects, the survival of neural stem cells and/or progenitor cells is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, or at least about 300% or more compared to a reference (e.g., subjects that did not receive an administration of the miR-485 inhibitor).

In some aspects, increasing and/or inducing neurogenesis is associated with an increased number of neural stem cells and/or progenitor cells. In certain aspects, the number of neural stem cells and/or progenitor cells is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, or at least about 300% or more compared to a reference (e.g., subjects that did not receive an administration of the miR-485 inhibitor).

In some aspects, increasing and/or inducing neurogenesis is associated with increased axon, dendrite, and/or synapse development. In certain aspects, axon, dendrite, and/or synapse development is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, or at least about 300% or more compared to a reference (e.g., subjects that did not receive an administration of the miR-485 inhibitor).

In some aspects, administering a miR-485 inhibitor disclosed herein prevents and/or inhibits the development of an amyloid beta plaque load in a subject (e.g., suffering from a neurodegenerative disease). In some aspects, administering a miR-485 inhibitor disclosed herein delays the onset of the development of an amyloid beta plaque load in a subject (e.g., suffering from a neurodegenerative disease). In some aspects, administering a miR-485 inhibitor of the present disclosure lowers the risk of development an amyloid beta plaque load in a subject (e.g., suffering from a neurodegenerative disease).

In some aspects, administering a miR-485 inhibitor of the present disclosure increases dendritic spine density of a neuron in a subject (e.g., suffering from a neurodegenerative disease) compared to a reference (e.g., dendritic spine density of a neuron in the subject prior to the administering). In some aspects, administering a miR-485 inhibitor of the present disclosure increases dendritic spine density of a neuron in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 150%, at least about 200%, at least about 250%, or at least about 300% or more compared to a reference (e.g., subjects that did not receive an administration of the miR-485 inhibitor).

In some aspects, administering a miR-485 inhibitor disclosed herein decreases the loss of dendritic spines of a neuron in a subject (e.g., suffering from a neurodegenerative disease) compared to a reference (e.g., loss of dendritic spines of a neuron in the subject prior to the administering). In certain aspects, administering a miR-485 inhibitor decreases the loss of dendritic spines of a neuron in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a reference (e.g., subjects that did not receive an administration of the miR-485 inhibitor).

In some aspects, administering a miR-485 inhibitor of the present disclosure decreases neuroinflammation (e.g., by increasing the expression of SIRT1 protein and/or SIRT1 gene) in a subject (e.g., suffering from a neurodegenerative disease) compared to a reference (e.g., neuroinflammation in the subject prior to the administering). In certain aspects, administering a miR-485 inhibitor decreases neuroinflammation in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a reference (e.g., subjects that did not receive an administration of the miR-485 inhibitor). In some aspects, decreased neuroinflammation comprises glial cells producing decreased amounts of inflammatory mediators. Accordingly, in certain aspects, administering a miR-485 inhibitor disclosed herein to a subject (e.g., suffering from a neurodegenerative disease) decreases the amount of inflammatory mediators produced by glial cells by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a reference (e.g., subjects that did not receive an administration of the miR-485 inhibitor). In some aspects, an inflammatory mediator produced by glial cells comprises TNF-α. In some aspects, the inflammatory mediator comprises IL-1β. In some aspects, an inflammatory mediator produced by glial cells comprises both TNF-α and IL-1β.

In some aspects, administering a miR-485 inhibitor disclosed herein increases autophagy (e.g., by increasing the expression of a SIRT1 protein and/or SIRT1 gene) in a subject (e.g., suffering from a neurodegenerative disease). As used herein, the term “autophagy” refers to cellular stress response and a survival pathway that is responsible for the degradation of long-lived proteins, protein aggregates, as well as damaged organelles in order to maintain cellular homeostasis. Not surprisingly, abnormalities of autophagy have been associated with number of diseases, including many neurodegenerative diseases (e.g., Alzheimer's disease and Parkinson's disease). In some aspects, administering a miR-485 inhibitor disclosed herein to a subject (e.g., suffering from a degenerative disease) increases autophagy by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 150%, at least about 200%, or at least about 300% or more, compared to a reference (e.g., subjects that did not receive an administration of the miR-485 inhibitor).

As is known in the art, many neurodegenerative diseases exhibit certain motor and/or non-motor symptoms. For instance, non-limiting examples of motor symptoms associated with Parkinson's disease include resting tremor, reduction of spontaneous movement (bradykinesia), rigidity, postural instability, freezing of gait, impaired handwriting (micrographia), decreased facial expression, and uncontrolled rapid movements. Non-limiting examples of non-motor symptoms associated with Parkinson's disease include autonomic dysfunction, neuropsychiatric problems (mood, cognition, behavior, or thought alterations), sensory alterations (especially altered sense of smell), and sleep difficulties.

In some aspects, administering a miR-485 inhibitor of the present disclosure improves one or more motor symptoms in a subject (e.g., suffering from a neurodegenerative disease) compared to a reference (e.g., corresponding motor symptoms in the subject prior to the administering). In certain aspects, administering a miR-485 inhibitor of the present disclosure improves one or more motor symptoms in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 150%, at least about 200%, at least about 250%, or at least about 300% or more compared to a reference (e.g., subjects that did not receive an administration of the miR-485 inhibitor).

In some aspects, administering a miR-485 inhibitor of the present disclosure improves one or more non-motor symptoms in a subject (e.g., suffering from a neurodegenerative disease) compared to a reference (e.g., corresponding non-motor symptom in the subject prior to the administering). In certain aspects, administering a miR-485 inhibitor disclosed herein improves one or more non-motor symptoms in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 150%, at least about 200%, at least about 250%, or at least about 300% or more compared to a reference (e.g., subjects that did not receive an administration of the miR-485 inhibitor).

In some aspects, administering a miR-485 inhibitor disclosed herein improves synaptic function in a subject (e.g., suffering from a neurodegenerative disease) compared to a reference (e.g., synaptic function in the subject prior to the administering). As used herein, the term “synaptic function,” refers to the ability of the synapse of a cell (e.g., a neuron) to pass an electrical or chemical signal to another cell (e.g., a neuron). In some aspects, administering a miR-485 inhibitor of the present disclosure improves synaptic function in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 150%, at least about 200%, at least about 250%, or at least about 300% or more compared to a reference (e.g., subjects that did not receive an administration of the miR-485 inhibitor).

In some aspects, administering a miR-485 inhibitor of the present disclosure can prevent, delay, and/or ameliorate the loss of synaptic function in a subject (e.g., suffering from a neurodegenerative disease) compared to a reference (e.g., loss of synaptic function in the subject prior to the administering). In some aspects, administering a miR-485 inhibitor prevents, delays, and/or ameliorates the loss of synaptic function in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a reference (e.g., subjects that did not receive an administration of the miR-485 inhibitor).

In some aspects, a miR-485 inhibitor disclosed herein can be administered by any suitable route known in the art. In certain aspects, a miR-485 inhibitor is administered intranasally, parenthetically, intramuscularly, subcutaneously, ophthalmic, intravenously, intraperitoneally, intradermally, intraorbitally, intracerebrally, intracranially, intracerebroventricularly, intraspinally, intraventricular, intrathecally, intracistemally, intracapsularly, intratumorally, or any combination thereof.

In some aspects, a miR-485 inhibitor of the present disclosure can be used in combination with one or more additional therapeutic agents. In some aspects, the additional therapeutic agent and the miR-485 inhibitor are administered concurrently. In certain aspects, the additional therapeutic agent and the miR-485 inhibitor are administered sequentially.

In some aspects, the administration of a miR-485 inhibitor disclosed herein does not result in any adverse effects. In certain aspects, miR-485 inhibitors of the present disclosure do not adversely affect body weight when administered to a subject. In some aspects, miR-485 inhibitors disclosed herein do not result in increased mortality or cause pathological abnormalities when administered to a subject.

IV. miRNA-485 Inhibitors Useful for the Present Disclosure

Disclosed herein are compounds that can inhibit miR-485 activity (miR-485 inhibitor). In some aspects, a miR-485 inhibitor of the present disclosure comprises a nucleotide sequence encoding a nucleotide molecule that comprises at least one miR-485 binding site, wherein the nucleotide molecule does not encode a protein. As described herein, in some aspects, the miR-485 binding site is at least partially complementary to the target miRNA nucleic acid sequence (i.e., miR-485), such that the miR-485 inhibitor hybridizes to the miR-485 nucleic acid sequence.

In some aspects, the miR-485 binding site of a miR inhibitor disclosed herein has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence complementarity to the nucleic acid sequence of a miR-485. In certain aspects, the miR-485 binding site is fully complementary to the nucleic acid sequence of a miR-485.

The miR-485 hairpin precursor can generate both miR-485-5p and miR-485-3p. In the context of the present disclosure “miR-485” encompasses both miR-485-5p and miR-485-3p unless specified otherwise. The human mature miR-485-3p has the sequence 5′-GUCAUACACGGCUCUCCUCUCU-3′ (SEQ ID NO: 1; miRBase Acc. No. MIMAT0002176). A 5′ terminal subsequence of miR-485-3p 5′-UCAUACA-3′ (SEQ ID NO: 49) is the seed sequence. The human mature miR-485-5p has the sequence 5′-AGAGGCUGGCCGUGAUGAAUUC-3′ (SEQ ID NO: 33; miRBase Acc. No. MIMAT0002175). A 5′ terminal subsequence of miR-485-5p 5′-GAGGCUG-3′ (SEQ ID NO: 50) is the seed sequence.

As will be apparent to those in the art, the human mature miR-485-3p has significant sequence similarity to that of other species. For instance, the mouse mature miR-485-3p differs from the human mature miR-485-3p by a single amino acid at each of the 5′- and 3′-ends (i.e., has an extra “A” at the 5′-end and missing “C” at the 3′-end). The mouse mature miR-485-3p has the following sequence:

5′-AGUCAUACACGGCUCUCCUCUC-3′ (SEQ ID NO: 34;
miRBase Acc. No. MIMAT0003129;

underlined portion corresponds to overlap to human mature miR-485-3p). The sequence for the mouse mature miR-485-5p is identical to that of the human: 5′-agaggcuggccgugaugaauuc-3′ (SEQ ID NO: 33; miRBase Acc. No. MIMAT0003128). Because of the similarity in sequences, in some aspects, a miR-485 inhibitor of the present disclosure is capable of binding miR-485-3p and/or miR-485-5p from one or more species. In certain aspects, a miR-485 inhibitor disclosed herein is capable of binding to miR-485-3p and/or miR-485-5p from both human and mouse.

In some aspects, the miR-485 binding site is a single-stranded polynucleotide sequence that is complementary (e.g., fully complementary) to a sequence of a miR-485-3p (or a subsequence thereof). In some aspects, the miR-485-3p subsequence comprises the seed sequence. Accordingly, in certain aspects, the miR-485 binding site has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence complementarity to the nucleic acid sequence set forth in SEQ ID NO: 49. In certain aspects, the miR-485 binding site is complementary to miR-485-3p except for 1, 2, 3, 4, 5, 6, 7, 8, 9, or 10 mismatches. In further aspects, the miR-485 binding site is fully complementary to the nucleic acid sequence set forth in SEQ ID NO: 1.

In some aspects, the miR-485 binding site is a single-stranded polynucleotide sequence that is complementary (e.g., fully complementary) to a sequence of a miR-485-5p (or a subsequence thereof). In some aspects, the miR-485-5p subsequence comprises the seed sequence. In certain aspects, the miR-485 binding site has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence complementarity to the nucleic acid sequence set forth in SEQ ID NO: 50. In certain aspects, the miR-485 binding site is complementary to miR-485-5p except for 1, 2, 3, 4, 5, 6, 7, 8, 9, or 10 mismatches. In further aspects, the miR-485 binding site is fully complementary to the nucleic acid sequence set forth in SEQ ID NO: 33.

The seed region of a miRNA forms a tight duplex with the target mRNA. Most miRNAs imperfectly base-pair with the 3′ untranslated region (UTR) of target mRNAs, and the 5′ proximal “seed” region of miRNAs provides most of the pairing specificity. Without being bound to any theory, it is believed that the first nine miRNA nucleotides (encompassing the seed sequence) provide greater specificity whereas the miRNA ribonucleotides 3′ of this region allow for lower sequence specificity and thus tolerate a higher degree of mismatched base pairing, with positions 2-7 being the most important. Accordingly, in specific aspects of the present disclosure, the miR-485 binding site comprises a subsequence that is fully complementary (i.e., 100% complementary) over the entire length of the seed sequence of miR-485.

miRNA sequences and miRNA binding sequences that can be used in the context of the disclosure include, but are not limited to, all or a portion of those sequences in the sequence listing provided herein, as well as the miRNA precursor sequence, or complement of one or more of these miRNAs. Any aspects of the disclosure involving specific miRNAs or miRNA binding sites by name is contemplated also to cover miRNAs or complementary sequences thereof whose sequences are at least about at least about 50%, at least about 55%, at least about 60%, at least about 65%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to the mature sequence of the specified miRNA sequence or complementary sequence thereof.

In some aspects, miRNA binding sequences of the present disclosure can include additional nucleotides at the 5′, 3′, or both 5′ and 3′ ends of those sequences in the sequence listing provided herein, as long as the modified sequence is still capable of specifically binding to miR-485. In some aspects, miRNA binding sequences of the present disclosure can differ in at least 1, 2, 3, 4, 5, 6, 7, 8, 9, 10 or more nucleotides with respect to those sequence in the sequence listing provided, as long as the modified sequence is still capable of specifically binding to miR-485.

It is also specifically contemplated that any methods and compositions discussed herein with respect to miRNA binding molecules or miRNA can be implemented with respect to synthetic miRNAs binding molecules. It is also understood that the disclosures related to RNA sequences in the present disclosure are equally applicable to corresponding DNA sequences.

In some aspects, a miRNA-485 inhibitor of the present disclosure comprises at least about 1 nucleotide, at least about 2 nucleotides, at least about 3 nucleotides, at least about 4 nucleotides, at least about 5 nucleotides, at least about 6 nucleotides, at least about 7 nucleotides, at least about 8 nucleotides, at least about 9 nucleotides, at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, or at least about 20 nucleotides at the 5′ of the nucleotide sequence. In some aspects, a miRNA-485 inhibitor comprises at least about 1 nucleotide, at least about 2 nucleotides, at least about 3 nucleotides, at least about 4 nucleotides, at least about 5 nucleotides, at least about 6 nucleotides, at least about 7 nucleotides, at least about 8 nucleotides, at least about 9 nucleotides, at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, or at least about 20 nucleotides at the 3′ of the nucleotide sequence.

In some aspects, a miR-485 inhibitor disclosed herein is about 6 to about 30 nucleotides in length. In certain aspects, a miR-485 inhibitor disclosed herein is about 7 nucleotides in length. In further aspects, a miR-485 inhibitor disclosed herein is about 8 nucleotides in length. In some aspects, a miR-485 inhibitor is about 9 nucleotides in length. In some aspects, a miR-485 inhibitor of the present disclosure is about 10 nucleotides in length. In certain aspects, a miR-485 inhibitor is about 11 nucleotides in length. In further aspects, a miR-485 inhibitor is about 12 nucleotides in length. In some aspects, a miR-485 inhibitor disclosed herein is about 13 nucleotides in length. In certain aspects, a miR-485 inhibitor disclosed herein is about 14 nucleotides in length. In some aspects, a miR-485 inhibitor disclosed herein is about 15 nucleotides in length. In further aspects, a miR-485 inhibitor is about 16 nucleotides in length. In certain aspects, a miR-485 inhibitor of the present disclosure is about 17 nucleotides in length. In some aspects, a miR-485 inhibitor is about 18 nucleotides in length. In some aspects, a miR-485 inhibitor is about 19 nucleotides in length. In certain aspects, a miR-485 inhibitor is about 20 nucleotides in length. In further aspects, a miR-485 inhibitor of the present disclosure is about 21 nucleotides in length. In some aspects, a miR-485 inhibitor is about 22 nucleotides in length.

In some aspects, a miR-485 inhibitor disclosed herein comprises a nucleotide sequence that is at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to a sequence selected from SEQ ID NOs: 2 to 30. In certain aspects, a miR-485 inhibitor comprises a nucleotide sequence selected from the group consisting of SEQ ID NOs: 2 to 30, wherein the nucleotide sequence can optionally comprise 1, 2, 3, 4, 5, 6, 7, 8, 9, or 10 mismatches.

In some aspects, a miRNA inhibitor comprises 5′-UGUAUGA-3′ (SEQ ID NO: 2), 5′-GUGUAUGA-3′ (SEQ ID NO: 3), 5′-CGUGUAUGA-3′ (SEQ ID NO: 4), 5′-CCGUGUAUGA-3′ (SEQ ID NO: 5), 5′-GCCGUGUAUGA-3′ (SEQ ID NO: 6), 5′-AGCCGUGUAUGA-3′ (SEQ ID NO: 7), 5′-GAGCCGUGUAUGA-3′ (SEQ ID NO: 8), 5′-AGAGCCGUGUAUGA-3′ (SEQ ID NO: 9), 5′-GAGAGCCGUGUAUGA-3′ (SEQ ID NO: 10), 5′-GGAGAGCCGUGUAUGA-3′ (SEQ ID NO: 11), 5′-AGGAGAGCCGUGUAUGA-3′ (SEQ ID NO: 12), 5′-GAGGAGAGCCGUGUAUGA-3′ (SEQ ID NO: 13), 5′-AGAGGAGAGCCGUGUAUGA-3′ (SEQ ID NO: 14), or 5′-GAGAGGAGAGCCGUGUAUGA-3′ (SEQ ID NO: 15).

In some aspects, the miRNA inhibitor has 5′-UGUAUGAC-3′ (SEQ ID NO: 16), 5′-GUGUAUGAC-3′ (SEQ ID NO: 17), 5′-CGUGUAUGAC-3′ (SEQ ID NO: 18), 5′-CCGUGUAUGAC-3′ (SEQ ID NO: 19), 5′-GCCGUGUAUGAC-3′ (SEQ ID NO: 20), 5′-AGCCGUGUAUGAC-3′ (SEQ ID NO: 21), 5′-GAGCCGUGUAUGAC-3′ (SEQ ID NO: 22), 5′-AGAGCCGUGUAUGAC-3′ (SEQ ID NO: 23), 5′-GAGAGCCGUGUAUGAC-3′ (SEQ ID NO: 24), 5′-GGAGAGCCGUGUAUGAC-3′ (SEQ ID NO: 25), 5′-AGGAGAGCCGUGUAUGAC-3′ (SEQ ID NO: 26), 5′-GAGGAGAGCCGUGUAUGAC-3′ (SEQ ID NO: 27), 5′-AGAGGAGAGCCGUGUAUGAC-3′ (SEQ ID NO: 28), 5′-GAGAGGAGAGCCGUGUAUGAC-3′ (SEQ ID NO: 29), or 5′-AGAGAGGAGAGCCGUGUAUGAC-3′ (SEQ ID NO: 30).

In some aspects, the miRNA inhibitor has a sequence selected from the group consisting of: 5′-TGTATGA-3′ (SEQ ID NO: 62), 5′-GTGTATGA-3′ (SEQ ID NO: 63), 5′-CGTGTATGA-3′ (SEQ ID NO: 64), 5′-CCGTGTATGA-3′ (SEQ ID NO: 65), 5′-GCCGTGTATGA-3′ (SEQ ID NO: 66), 5′-AGCCGTGTATGA-3′ (SEQ ID NO: 67), 5′-GAGCCGTGTATGA-3′ (SEQ ID NO: 68), 5′-AGAGCCGTGTATGA-3′ (SEQ ID NO: 69), 5′-GAGAGCCGTGTATGA-3′ (SEQ ID NO: 70), 5′-GGAGAGCCGTGTATGA-3′ (SEQ ID NO: 71), 5′-AGGAGAGCCGTGTATGA-3′ (SEQ ID NO: 72), 5′-GAGGAGAGCCGTGTATGA-3′ (SEQ ID NO: 73), 5′-AGAGGAGAGCCGTGTATGA-3′ (SEQ ID NO: 74), 5′-GAGAGGAGAGCCGTGTATGA-3′ (SEQ ID NO: 75); 5′-TGTATGAC-3′ (SEQ ID NO: 76), 5′-GTGTATGAC-3′ (SEQ ID NO: 77), 5′-CGTGTATGAC-3′ (SEQ ID NO: 78), 5′-CCGTGTATGAC-3′ (SEQ ID NO: 79), 5′-GCCGTGTATGAC-3′ (SEQ ID NO: 80), 5′-AGCCGTGTATGAC-3′ (SEQ ID NO: 81), 5′-GAGCCGTGTATGAC-3′ (SEQ ID NO: 82), 5′-AGAGCCGTGTATGAC-3′ (SEQ ID NO: 83), 5′-GAGAGCCGTGTATGAC-3′ (SEQ ID NO: 84), 5′-GGAGAGCCGTGTATGAC-3′ (SEQ ID NO: 85), 5′-AGGAGAGCCGTGTATGAC-3′ (SEQ ID NO: 86), 5′-GAGGAGAGCCGTGTATGAC-3′ (SEQ ID NO: 87), 5′-AGAGGAGAGCCGTGTATGAC-3′ (SEQ ID NO: 88), 5′-GAGAGGAGAGCCGTGTATGAC-3′ (SEQ ID NO: 89); and 5′-AGAGAGGAGAGCCGTGTATGAC-3′ (SEQ ID NO: 90).

In some aspects, a miRNA inhibitor disclosed herein (i.e., miR-485 inhibitor) comprises a nucleotide sequence that is at least about 50%, at least about 55%, at least about 60%, at least about 65%, at least about 70%, at least about 75%, at least about 80%, at least about 85%, at least about 90%, or at least about 95% identical to 5′-AGAGGAGAGCCGUGUAUGAC-3′ (SEQ ID NO: 28) or 5′-AGAGGAGAGCCGTGTATGAC-3′ (SEQ ID NO: 88). In some aspects, the miRNA inhibitor comprises a nucleotide sequence that has at least 90% similarity to 5′-AGAGGAGAGCCGUGUAUGAC-3′ (SEQ ID NO: 28) or 5′-AGAGGAGAGCCGTGTATGAC-3′ (SEQ ID NO: 88). In some aspects, the miRNA inhibitor comprises the nucleotide sequence 5′-AGAGGAGAGCCGUGUAUGAC-3′ (SEQ ID NO: 28) or 5′-AGAGGAGAGCCGTGTATGAC-3′ (SEQ ID NO: 88) with one substitution or two substitutions. In certain aspects, the miRNA inhibitor comprises the nucleotide sequence 5′-AGAGGAGAGCCGUGUAUGAC-3′ (SEQ ID NO: 28). In some aspects, the miRNA inhibitor comprises the nucleotide sequence 5′-AGAGGAGAGCCGTGTATGAC-3′ (SEQ ID NO: 88).

In some aspects, a miRNA inhibitor disclosed herein (i.e., miR-485 inhibitor) comprises a nucleotide sequence that is at least about 50%, at least about 55%, at least about 60%, at least about 65%, at least about 70%, at least about 75%, at least about 80%, at least about 85%, at least about 90%, or at least about 95% identical to 5′-AGAGAGGAGAGCCGUGUAUGAC-3′ (SEQ ID NO: 30) or 5′-AGAGAGGAGAGCCGTGTATGAC-3′ (SEQ ID NO: 90). In some aspects, the miRNA inhibitor comprises a nucleotide sequence that has at least 90% similarity to 5′-AGAGAGGAGAGCCGUGUAUGAC-3′ (SEQ ID NO: 30) or 5′-AGAGAGGAGAGCCGTGTATGAC-3′ (SEQ ID NO: 90). In some aspects, the miRNA inhibitor comprises the nucleotide sequence 5′-AGAGAGGAGAGCCGUGUAUGAC-3′ (SEQ ID NO: 30) or 5′-AGAGAGGAGAGCCGTGTATGAC-3′ (SEQ ID NO: 90) with one substitution or two substitutions. In certain aspects, the miRNA inhibitor comprises the nucleotide sequence 5′-AGAGAGGAGAGCCGUGUAUGAC-3′ (SEQ ID NO: 30). In some aspects, the miRNA inhibitor comprises the nucleotide sequence 5′-AGAGAGGAGAGCCGTGTATGAC-3′ (SEQ ID NO: 90).

In some aspects, a miR-485 inhibitor of the present disclosure comprises the sequence disclosed herein, e.g., any one of SEQ ID NOs: 2 to 30, and at least one, at least two, at least three, at least four or at least five additional nucleic acid at the N terminus, at least one, at least two, at least three, at least four, or at least five additional nucleic acid at the C terminus, or both. In some aspects, a miR-485 inhibitor of the present disclosure comprises the sequence disclosed herein, e.g., any one of SEQ ID NOs: 2 to 30, and one additional nucleic acid at the N terminus and/or one additional nucleic acid at the C terminus. In some aspects, a miR-485 inhibitor of the present disclosure comprises the sequence disclosed herein, e.g., any one of SEQ ID NOs: 2 to 30, and one or two additional nucleic acids at the N terminus and/or one or two additional nucleic acids at the C terminus. In some aspects, a miR-485 inhibitor of the present disclosure comprises the sequence disclosed herein, e.g., any one of SEQ ID NOs: 2 to 30, and one to three additional nucleic acids at the N terminus and/or one to three additional nucleic acids at the C terminus. In some aspects, a miR-485 inhibitor comprises 5′-GAGAGGAGAGCCGUGUAUGAC-3′ (SEQ ID NO: 29). In certain aspects, a miR-485 inhibitor comprises 5′-AGAGAGGAGAGCCGUGUAUGAC-3′ (SEQ ID NO: 30).

In some aspects, a miR-485 inhibitor of the present disclosure comprises one miR-485 binding site. In further aspects, a miR-485 inhibitor disclosed herein comprises at least two miR-485 binding sites. In certain aspects, a miR-485 inhibitor comprises three miR-485 binding sites. In some aspects, a miR-485 inhibitor comprises four miR-485 binding sites. In some aspects, a miR-485 inhibitor comprises five miR-485 binding sites. In certain aspects, a miR-485 inhibitor comprises six or more miR-485 binding sites. In some aspects, all the miR-485 binding sites are identical. In some aspects, all the miR-485 binding sites are different. In some aspects, at least one of the miR-485 binding sites is different. In some aspects, all the miR-485 binding sites are miR-485-3p binding sites. In other aspects, all the miR-485 binding sites are miR-485-5p binding sites. In further aspects, a miR-485 inhibitor comprises at least one miR-485-3p binding site and at least one miR-485-5p binding site.

IV a. Chemically Modified Polynucleotides

In some aspects, a miR-485 inhibitor disclosed herein comprises a polynucleotide which includes at least one chemically modified nucleoside and/or nucleotide. When the polynucleotides of the present disclosure are chemically modified the polynucleotides can be referred to as “modified polynucleotides.”

A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside including a phosphate group. Modified nucleotides can be synthesized by any useful method, such as, for example, chemically, enzymatically, or recombinantly, to include one or more modified or non-natural nucleosides.

Polynucleotides can comprise a region or regions of linked nucleosides. Such regions can have variable backbone linkages. The linkages can be standard phosphodiester linkages, in which case the polynucleotides would comprise regions of nucleotides.

The modified polynucleotides disclosed herein can comprise various distinct modifications. In some aspects, the modified polynucleotides contain one, two, or more (optionally different) nucleoside or nucleotide modifications. In some aspects, a modified polynucleotide can exhibit one or more desirable properties, e.g., improved thermal or chemical stability, reduced immunogenicity, reduced degradation, increased binding to the target microRNA, reduced non-specific binding to other microRNA or other molecules, as compared to an unmodified polynucleotide.

In some aspects, a polynucleotide of the present disclosure (e.g., a miR-485 inhibitor) is chemically modified. As used herein, in reference to a polynucleotide, the terms “chemical modification” or, as appropriate, “chemically modified” refer to modification with respect to adenosine (A), guanosine (G), uridine (U), thymidine (T) or cytidine (C) ribo- or deoxyribonucleosides in one or more of their position, pattern, percent or population, including, but not limited to, its nucleobase, sugar, backbone, or any combination thereof.

In some aspects, a polynucleotide of the present disclosure (e.g., a miR-485 inhibitor) can have a uniform chemical modification of all or any of the same nucleoside type or a population of modifications produced by downward titration of the same starting modification in all or any of the same nucleoside type, or a measured percent of a chemical modification of all any of the same nucleoside type but with random incorporation In further aspects, the polynucleotide of the present disclosure (e.g., a miR-485 inhibitor) can have a uniform chemical modification of two, three, or four of the same nucleoside type throughout the entire polynucleotide (such as all uridines and/or all cytidines, etc. are modified in the same way).

Modified nucleotide base pairing encompasses not only the standard adenine-thymine, adenine-uracil, or guanine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleobase inosine and adenine, cytosine or uracil. Any combination of base/sugar or linker can be incorporated into polynucleotides of the present disclosure.

The skilled artisan will appreciate that, except where otherwise noted, polynucleotide sequences set forth in the instant application will recite “T”s in a representative DNA sequence but where the sequence represents RNA, the “T”s would be substituted for “U”s. For example, TD's of the present disclosure can be administered as RNAs, as DNAs, or as hybrid molecules comprising both RNA and DNA units.

In some aspects, the polynucleotide (e.g., a miR-485 inhibitor) includes a combination of at least two (e.g., 2, 3, 4, 5, 6, 7, 8, 8, 10, 11, 12, 13, 14, 15, 16, 17, 18, 18, 20 or more) modified nucleobases.

In some aspects, the nucleobases, sugar, backbone linkages, or any combination thereof in a polynucleotide are modified by at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100%.

(i) Base Modification

In certain aspects, the chemical modification is at nucleobases in a polynucleotide of the present disclosure (e.g., a miR-485 inhibitor). In some aspects, the at least one chemically modified nucleoside is a modified uridine (e.g., pseudouridine (ψ), 2-thiouridine (s2U), 1-methyl-pseudouridine (m1ψ), 1-ethyl-pseudouridine (e1ψ), or 5-methoxy-uridine (mo5U)), a modified cytosine (e.g., 5-methyl-cytidine (m5C)) a modified adenosine (e.g, 1-methyl-adenosine (m1A), N6-methyl-adenosine (m6A), or 2-methyl-adenine (m2A)), a modified guanosine (e.g., 7-methyl-guanosine (m7G) or 1-methyl-guanosine (m1G)), or a combination thereof.

In some aspects, the polynucleotide of the present disclosure (e.g., a miR-485 inhibitor) is uniformly modified (e.g., fully modified, modified throughout the entire sequence) for a particular modification. For example, a polynucleotide can be uniformly modified with the same type of base modification, e.g., 5-methyl-cytidine (m5C), meaning that all cytosine residues in the polynucleotide sequence are replaced with 5-methyl-cytidine (m5C). Similarly, a polynucleotide can be uniformly modified for any type of nucleoside residue present in the sequence by replacement with a modified nucleoside such as any of those set forth above.

In some aspects, the polynucleotide of the present disclosure (e.g., a miR-485 inhibitor) includes a combination of at least two (e.g., 2, 3, 4 or more) of modified nucleobases. In some aspects, at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of a type of nucleobases in a polynucleotide of the present disclosure (e.g., a miR-485 inhibitor) are modified nucleobases.

(ii) Backbone Modifications

In some aspects, the polynucleotide of the present disclosure (i.e., miR-485 inhibitor) can include any useful linkage between the nucleosides. Such linkages, including backbone modifications, that are useful in the composition of the present disclosure include, but are not limited to the following: 3-alkylene phosphonates, 3′-amino phosphoramidate, alkene containing backbones, aminoalkylphosphoramidates, aminoalkylphosphotriesters, boranophosphates, —CH₂—O—N(CH₃)—CH₂—, —CH₂—N(CH₃)—N(CH₃)—CH₂—, —CH₂—NH—CH₂—, chiral phosphonates, chiral phosphorothioates, formacetyl and thioformacetyl backbones, methylene (methylimino), methylene formacetyl and thioformacetyl backbones, methyleneimino and methylenehydrazino backbones, morpholino linkages, —N(CH₃)—CH₂—CH₂—, oligonucleosides with heteroatom internucleoside linkage, phosphinates, phosphoramidates, phosphorodithioates, phosphorothioate internucleoside linkages, phosphorothioates, phosphotriesters, PNA, siloxane backbones, sulfamate backbones, sulfide sulfoxide and sulfone backbones, sulfonate and sulfonamide backbones, thionoalkylphosphonates, thionoalkylphosphotriesters, and thionophosphoramidates.

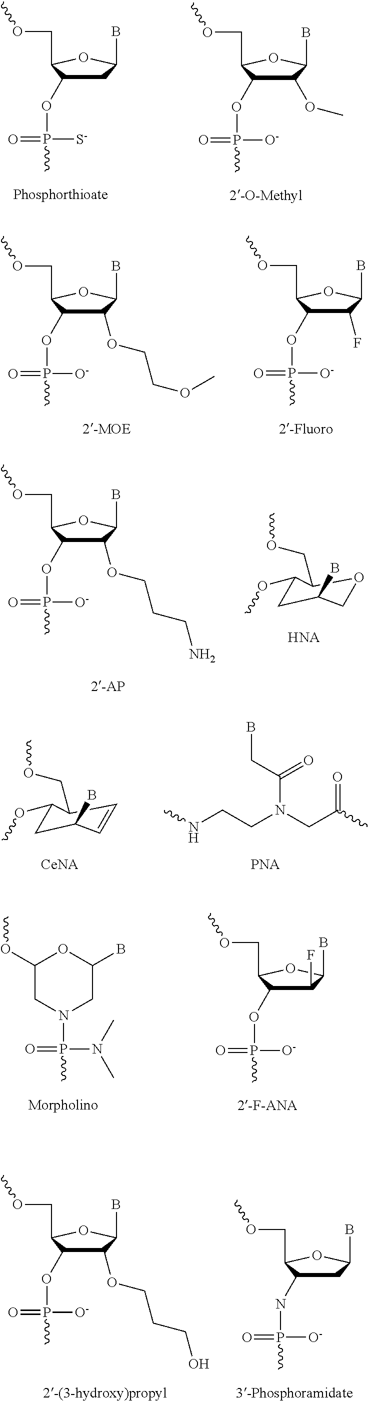

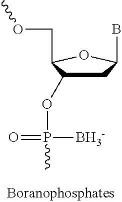

In some aspects, the presence of a backbone linkage disclosed above increase the stability and resistance to degradation of a polynucleotide of the present disclosure (i.e., miR-485 inhibitor).

In some aspects, at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of the backbone linkages in a polynucleotide of the present disclosure (i.e., miR-485 inhibitor) are modified (e.g., all of them are phosphorothioate).

In some aspects, a backbone modification that can be included in a polynucleotide of the present disclosure (i.e., miR-485 inhibitor) comprises phosphorodiamidate morpholino oligomer (PMO) and/or phosphorothioate (PS) modification.

(iii) Sugar Modifications

The modified nucleosides and nucleotides which can be incorporated into a polynucleotide of the present disclosure (i.e., miR-485 inhibitor) can be modified on the sugar of the nucleic acid. In some aspects, the sugar modification increases the affinity of the binding of a miR-485 inhibitor to miR-485 nucleic acid sequence. Incorporating affinity-enhancing nucleotide analogues in the miR-485 inhibitor, such as LNA or 2′-substituted sugars, can allow the length and/or the size of the miR-485 inhibitor to be reduced.

In some aspects, at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of the nucleotides in a polynucleotide of the present disclosure (i.e., miR-485 inhibitor) contain sugar modifications (e.g., LNA).

In some aspects, 1, 2, 3, 4, 5, 6, 7, 8, 9, 10, 11, 12, 13, 14, 15, 16, 17, 18, 19, 20, 21, or 22 nucleotide units in a polynucleotide of the present disclosure are sugar modified (e.g., LNA).

Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar.

The 2′ hydroxyl group (OH) of ribose can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C₁₋₆ alkylene or C₁₋₆ heteroalkylene bridge to the 4′-carbon of the same ribose sugar, where exemplary bridges include methylene, propylene, ether, amino bridges, aminoalkyl, aminoalkoxy, amino, and amino acid.

In some aspects, nucleotide analogues present in a polynucleotide of the present disclosure (i.e., mir-485 inhibitor) comprise, e.g., 2′-O-alkyl-RNA units, 2′-OMe-RNA units, 2′-O-alkyl-SNA, 2′-amino-DNA units, 2′-fluoro-DNA units, LNA units, arabino nucleic acid (ANA) units, 2′-fluoro-ANA units, HNA units, INA (intercalating nucleic acid) units, 2′MOE units, or any combination thereof. In some aspects, the LNA is, e.g., oxy-LNA (such as beta-D-oxy-LNA, or alpha-L-oxy-LNA), amino-LNA (such as beta-D-amino-LNA or alpha-L-amino-LNA), thio-LNA (such as beta-D-thio0-LNA or alpha-L-thio-LNA), ENA (such a beta-D-ENA or alpha-L-ENA), or any combination thereof. In further aspects, nucleotide analogues that can be included in a polynucleotide of the present disclosure (i.e., miR-485 inhibitor) comprises a locked nucleic acid (LNA), an unlocked nucleic acid (UNA), an arabino nucleic acid (ABA), a bridged nucleic acid (BNA), and/or a peptide nucleic acid (PNA).

In some aspects, a polynucleotide of the present disclosure (i.e., miR-485 inhibitor) can comprise both modified RNA nucleotide analogues (e.g., LNA) and DNA units. In some aspects, a miR-485 inhibitor is a gapmer. See, e.g., U.S. Pat. Nos. 8,404,649; 8,580,756; 8,163,708; 9,034,837; all of which are herein incorporated by reference in their entireties. In some aspects, a miR-485 inhibitor is a micromir. See U.S. Pat. Appl. Publ. No. US20180201928, which is herein incorporated by reference in its entirety.

In some aspects, a polynucleotide of the present disclosure (i.e., miR-485 inhibitor) can include modifications to prevent rapid degradation by endo- and exo-nucleases. Modifications include, but are not limited to, for example, (a) end modifications, e.g., 5′ end modifications (phosphorylation, dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages.

### V. Vectors and Delivery Systems

In some aspects, the miR-485 inhibitors of the present disclosure can be administered, e.g., to a subject suffering from a disease or condition associated with abnormal (e.g., reduced) level of a SIRT1 protein and/or SIRT1 gene, using any relevant delivery system known in the art. In certain aspects, the delivery system is a vector. Accordingly, in some aspects, the present disclosure provides a vector comprising a miR-485 inhibitor of the present disclosure.

In some aspects, the vector is viral vector. In some aspects, the viral vector is an adenoviral vector or an adenoassociated viral vector. In certain aspects, the viral vector is an AAV that has a serotype of AAV2, AAV3, AAV4, AAV5, AAV6, AAV7, AAV8, AAV9, AAV10, or any combination thereof. In some aspects, the adenoviral vector is a third generation adenoviral vector. ADEASY™ is by far the most popular method for creating adenoviral vector constructs. The system consists of two types of plasmids: shuttle (or transfer) vectors and adenoviral vectors. The transgene of interest is cloned into the shuttle vector, verified, and linearized with the restriction enzyme PmeI. This construct is then transformed into ADEASIER-1 cells, which are BJ5183 E. coli cells containing PADEASY™ PADEEASY™ is a ˜33 Kb adenoviral plasmid containing the adenoviral genes necessary for virus production. The shuttle vector and the adenoviral plasmid have matching left and right homology arms which facilitate homologous recombination of the transgene into the adenoviral plasmid. One can also co-transform standard BJ5183 with supercoiled PADEASY™ and the shuttle vector, but this method results in a higher background of non-recombinant adenoviral plasmids. Recombinant adenoviral plasmids are then verified for size and proper restriction digest patterns to determine that the transgene has been inserted into the adenoviral plasmid, and that other patterns of recombination have not occurred. Once verified, the recombinant plasmid is linearized with Pac to create a linear dsDNA construct flanked by ITRs. 293 or 911 cells are transfected with the linearized construct, and virus can be harvested about 7-10 days later. In addition to this method, other methods for creating adenoviral vector constructs known in the art at the time the present application was filed can be used to practice the methods disclosed herein.

In some aspects, the viral vector is a retroviral vector, e.g., a lentiviral vector (e.g., a third or fourth generation lentiviral vector). Lentiviral vectors are usually created in a transient transfection system in which a cell line is transfected with three separate plasmid expression systems. These include the transfer vector plasmid (portions of the HIV provirus), the packaging plasmid or construct, and a plasmid with the heterologous envelop gene (env) of a different virus. The three plasmid components of the vector are put into a packaging cell which is then inserted into the HIV shell. The virus portions of the vector contain insert sequences so that the virus cannot replicate inside the cell system. Current third generation lentiviral vectors encode only three of the nine HIV-1 proteins (Gag, Pol, Rev), which are expressed from separate plasmids to avoid recombination-mediated generation of a replication-competent virus. In fourth generation lentiviral vectors, the retroviral genome has been further reduced (see, e.g., TAKARA® LENTI-X™ fourth-generation packaging systems).

Any AAV vector known in the art can be used in the methods disclosed herein. The AAV vector can comprise a known vector or can comprise a variant, fragment, or fusion thereof. In some aspects, the AAV vector is selected from the group consisting of AAV type 1 (AAV1), AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, bovine AAV, shrimp AVV, snake AVV, and any combination thereof.

In some aspects, the AAV vector is derived from an AAV vector selected from the group consisting of AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof.

In some aspects, the AAV vector is a chimeric vector derived from at least two AAV vectors selected from the group consisting of AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof.

In certain aspects, the AAV vector comprises regions of at least two different AAV vectors known in the art.

In some aspects, the AAV vector comprises an inverted terminal repeat from a first AAV (e.g., AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, or any derivative thereof) and a second inverted terminal repeat from a second AAV (e.g., AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, or any derivative thereof).

In some aspects, the AVV vector comprises a portion of an AAV vector selected from the group consisting of AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof. In some aspects, the AAV vector comprises AAV2.

In some aspects, the AVV vector comprises a splice acceptor site. In some aspects, the AVV vector comprises a promoter. Any promoter known in the art can be used in the AAV vector of the present disclosure. In some aspects, the promoter is an RNA Pol III promoter. In some aspects, the RNA Pol III promoter is selected from the group consisting of the U6 promoter, the H1 promoter, the 7SK promoter, the 5S promoter, the adenovirus 2 (Ad2) VAI promoter, and any combination thereof. In some aspects, the promoter is a cytomegalovirus immediate-early gene (CMV) promoter, an EF1a promoter, an SV40 promoter, a PGK1 promoter, a Ubc promoter, a human beta actin promoter, a CAG promoter, a TRE promoter, a UAS promoter, a Ac5 promoter, a polyhedrin promoter, a CaMKIIa promoter, a GAL1 promoter, a GAL10 promoter, a TEF promoter, a GDS promoter, a ADH1 promoter, a CaMV35S promoter, or a Ubi promoter. In a specific aspect, the promoter comprises the U6 promoter.

In some aspects, the AAV vector comprises a constitutively active promoter (constitutive promoter). In some aspects, the constitutive promoter is selected from the group consisting of hypoxanthine phosphoribosyl transferase (HPRT), adenosine deaminase, pyruvate kinase, beta-actin promoter, cytomegalovirus (CMV), simian virus (e.g., SV40), papilloma virus, adenovirus, human immunodeficiency virus (HIV), Rous sarcoma virus, a retrovirus long terminal repeat (LTR), Murine stem cell virus (MSCV) and the thymidine kinase promoter of herpes simplex virus.

In some aspects, the promoter is an inducible promoter. In some aspects, the inducible promoter is a tissue specific promoter. In certain aspects, the tissue specific promoter drives transcription of the coding region of the AVV vector in a neuron, a glial cell, or in both a neuron and a glial cell.

In some aspects, the AVV vector comprises one or more enhancers. In some aspects, the one or more enhancer are present in the AAV alone or together with a promoter disclosed herein. In some aspects, the AAV vector comprises a 3′UTR poly(A) tail sequence. In some aspects, the 3′UTR poly(A) tail sequence is selected from the group consisting of bGH poly(A), actin poly(A), hemoglobin poly(A), and any combination thereof. In some aspects, the 3′UTR poly(A) tail sequence comprises bGH poly(A).

In some aspects, a miR-485 inhibitor disclosed herein is administered with a delivery agent. Non-limiting examples of delivery agents that can be used include an exosome, a lipidoid, a liposome, a lipoplex, a lipid nanoparticle, an extracellular vesicle, a synthetic vesicle, a polymeric compound, a peptide, a protein, a cell, a nanoparticle mimic, a nanotube, a micelle, a viral vector, or a conjugate.

Thus, in some aspects, the present disclosure also provides a composition comprising a miRNA inhibitor of the present disclosure (i.e., miR-485 inhibitor) and a delivery agent. In some aspects, the delivery agent comprises a cationic carrier unit comprising

[WP]-L1-[CC]-L2-[AM] (formula I)

or

[WP]-L1-[AM]-L2-[CC] (formula II)

wherein
WP is a water-soluble biopolymer moiety;
CC is a positively charged (i.e., cationic) carrier moiety;
AM is an adjuvant moiety; and,
L1 and L2 are independently optional linkers, and
wherein when mixed with a nucleic acid at an ionic ratio of about 1:1, the cationic carrier unit forms a micelle. Accordingly, in some aspects, the miRNA inhibitor and the cationic carrier unit are capable of associating with each other (e.g., via a covalent bond or a non-valent bond) to form a micelle when mixed together.

In some aspects, composition comprising a miRNA inhibitor of the present disclosure (i.e., miR-485 inhibitor) interacts with the cationic carrier unit via an ionic bond.

In some aspects, the water-soluble polymer comprises poly(alkylene glycols), poly(oxyethylated polyol), poly(olefinic alcohol), poly(vinylpyrrolidone), poly(hydroxyalkylmethacrylamide), poly(hydroxyalkylmethacrylate), poly(saccharides), poly(v-hydroxy acid), poly(vinyl alcohol), polyglycerol, polyphosphazene, polyoxazolines (“POZ”) poly(N-acryloylmorpholine), or any combinations thereof. In some aspects, the water-soluble polymer comprises polyethylene glycol (“PEG”), polyglycerol, or poly(propylene glycol) (“PPG”). In some aspects, the water-soluble polymer comprises:

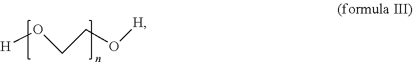

wherein n is 1-1000.

In some aspects, the n is at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, or at least about 141. In some aspects, the n is about 80 to about 90, about 90 to about 100, about 100 to about 110, about 110 to about 120, about 120 to about 130, about 140 to about 150, about 150 to about 160.

In some aspects, the water-soluble polymer is linear, branched, or dendritic. In some aspects, the cationic carrier moiety comprises one or more basic amino acids. In some aspects, the cationic carrier moiety comprises at least three, at least four, at least five, at least six, at least seven, at least eight, at least nine, at least ten, at least 11, at least 12, at least 13, at least 14, at last 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 basic amino acids. In some aspects, the cationic carrier moiety comprises about 30 to about 50 basic amino acids. In some aspects, the basic amino acid comprises arginine, lysine, histidine, or any combination thereof. In some aspects, the cationic carrier moiety comprises about 40 lysine monomers.

In some aspects, the adjuvant moiety is capable of modulating an immune response, an inflammatory response, and/or a tissue microenvironment. In some aspects, the adjuvant moiety comprises an imidazole derivative, an amino acid, a vitamin, or any combination thereof. In some aspects, the adjuvant moiety comprises:

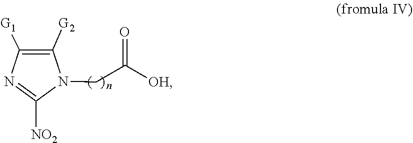

wherein each of G1 and G2 is H, an aromatic ring, or 1-10 alkyl, or G1 and G2 together form an aromatic ring, and wherein n is 1-10.

In some aspects, the adjuvant moiety comprises nitroimidazole. In some aspects, the adjuvant moiety comprises metronidazole, tinidazole, nimorazole, dimetridazole, pretomanid, ornidazole, megazol, azanidazole, benznidazole, or any combination thereof. In some aspects, the adjuvant moiety comprises an amino acid.

In some aspects, the adjuvant moiety comprises

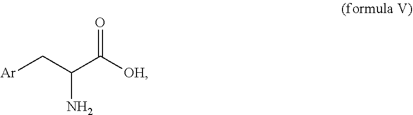

wherein Ar is

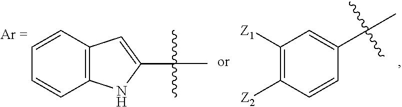

and
wherein each of Z1 and Z2 is H or OH.

In some aspects, the adjuvant moiety comprises a vitamin. In some aspects, the vitamin comprises a cyclic ring or cyclic hetero atom ring and a carboxyl group or hydroxyl group. In some aspects, the vitamin comprises:

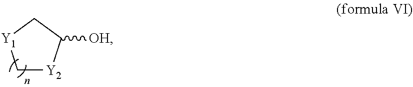

wherein each of Y1 and Y2 is C, N, O, or S, and wherein n is 1 or 2.

In some aspects, the vitamin is selected from the group consisting of vitamin A, vitamin B1, vitamin B2, vitamin B3, vitamin B6, vitamin B7, vitamin B9, vitamin B12, vitamin C, vitamin D2, vitamin D3, vitamin E, vitamin M, vitamin H, and any combination thereof. In some aspects, the vitamin is vitamin B3.

In some aspects, the adjuvant moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, or at least about 20 vitamin B3. In some aspects, the adjuvant moiety comprises about 10 vitamin B3.

In some aspects, the composition comprises a water-soluble biopolymer moiety with about 120 to about 130 PEG units, a cationic carrier moiety comprising a poly-lysine with about 30 to about 40 lysines, and an adjuvant moiety with about 5 to about 10 vitamin B3.

In some aspects, the composition comprises (i) a water-soluble biopolymer moiety with about 100 to about 200 PEG units, (ii) about 30 to about 40 lysines with an amine group (e.g., about 32 lysines), (iii) about 15 to 20 lysines, each having a thiol group (e.g., about 16 lysines, each with a thiol group), and (iv) about 30 to 40 lysines fused to vitamin B3 (e.g., about 32 lysines, each fused to vitamin B3). In some aspects, the composition further comprises a targeting moiety, e.g., a LAT1 targeting ligand, e.g., phenyl alanine, linked to the water soluble polymer. In some aspects, the thiol groups in the composition form disulfide bonds.

In some aspects, the composition comprises (1) a micelle comprising (i) about 100 to about 200 PEG units, (ii) about 30 to about 40 lysines with an amine group (e.g., about 32 lysines), (iii) about 15 to 20 lysines, each having a thiol group (e.g., about 16 lysines, each with a thiol group), and (iv) about 30 to 40 lysines fused to vitamin B3 (e.g., about 32 lysines, each fused to vitamin B3), and (2) a miR485 inhibitor (e.g., SEQ ID NO: 30), wherein the miR485 inhibitor is encapsulated within the micelle. In some aspects, the composition further comprises a targeting moiety, e.g., a LAT1 targeting ligand, e.g., phenyl alanine, linked to the PEG units. In some aspects, the thiol groups in the micelle form disulfide bonds.

The present disclosure also provides a micelle comprising a miRNA inhibitor of the present disclosure (i.e., miR-485 inhibitor) wherein the miRNA inhibitor and the delivery agent are associated with each other.

In some aspects, the association is a covalent bond, a non-covalent bond, or an ionic bond. In some aspects, the positive charge of the cationic carrier moiety of the cationic carrier unit is sufficient to form a micelle when mixed with the miR-485 inhibitor disclosed herein in a solution, wherein the overall ionic ratio of the positive charges of the cationic carrier moiety of the cationic carrier unit and the negative charges of the miR-485 inhibitor (or vector comprising the inhibitor) in the solution is about 1:1.

In some aspects, the cationic carrier unit is capable of protecting the miRNA inhibitor of the present disclosure (i.e., miR-485 inhibitor) from enzymatic degradation. See U.S. PCT Publication No. WO2020/261227, published Dec. 30, 2020, which is herein incorporated by reference in its entirety.

### VI. Pharmaceutical Compositions

In some aspects, the present disclosure also provides pharmaceutical compositions comprising a miR-485 inhibitor disclosed herein (e.g., a polynucleotide or a vector comprising the miR-485 inhibitor) that are suitable for administration to a subject. The pharmaceutical compositions generally comprise a miR-485 inhibitor described herein (e.g., a polynucleotide or a vector) and a pharmaceutically-acceptable excipient or carrier in a form suitable for administration to a subject. Pharmaceutically acceptable excipients or carriers are determined in part by the particular composition being administered, as well as by the particular method used to administer the composition.

Accordingly, there is a wide variety of suitable formulations of pharmaceutical compositions comprising a miR-485 inhibitor of the present disclosure. (See, e.g., Remington's Pharmaceutical Sciences, Mack Publishing Co., Easton, Pa. 18th ed. (1990)). The pharmaceutical compositions are generally formulated sterile and in full compliance with all Good Manufacturing Practice (GMP) regulations of the U.S. Food and Drug Administration.

### VII. Kits

The present disclosure also provides kits or products of manufacture, comprising a miRNA inhibitor of the present disclosure (e.g., a polynucleotide, vector, or pharmaceutical composition disclosed herein) and optionally instructions for use, e.g., instructions for use according to the methods disclosed herein. In some aspects, the kit or product of manufacture comprises a miR-485 inhibitor (e.g., vector, e.g., an AAV vector, a polynucleotide, or a pharmaceutical composition of the present disclosure) in one or more containers. In some aspects, the kit or product of manufacture comprises miR-485 inhibitor (e.g., a vector, e.g., an AAV vector, a polynucleotide, or a pharmaceutical composition of the present disclosure) and a brochure. One skilled in the art will readily recognize that miR-485 inhibitors disclosed herein (e.g., vectors, polynucleotides, and pharmaceutical compositions of the present disclosure, or combinations thereof) can be readily incorporated into one of the established kit formats which are well known in the art.

The following examples are offered by way of illustration and not by way of limitation.

### EXAMPLES

### Example 1: Materials and Methods

The Examples described below use one or more of the following materials and methods:

### Human Tissue

Brain precentral gyrus samples from patients with Alzheimer's disease (AD) and from controls were purchased from Netherlands brain bank. Information related to these patients and controls are shown in Table 1.

### Mice

B6SJLF1/J (JAX #100012), and five familial AD mutation (5XFAD) transgenic mice (#MMRRC #034848) were purchased from The Jackson Laboratory (Bar Harbor, Me., USA). 5XFAD mice overexpress mutant human amyloid precursor protein (APP) with the Swedish (K670N, M671L), Florida (I716V), and London (V717I) mutations, along with mutant human presenilin 1 (PS1) that carries two FAD mutations (M146L and L286V). These transgenes are regulated by the Thy1 promoter in neurons. The genotype of 5XFAD mice was confirmed by PCR analysis of tail DNA following standard PCR conditions provided by The Jackson Laboratory. Mice of mixed genotypes were housed four to five per cage with a 12-hour light/12-hour dark cycle and food and water ad libitum. All animal procedures were performed according to the Konyang University guidelines for care and use of laboratory animals. The animal studies were approved by the Konyang University Committee (Permit number: P-18-18-A-01).

### Next Generation Sequencing Using Mouse Frontal Cortex Tissue

NGS was performed in a NovaSeq 6000 system (Illumina, https://www.illumina.com/) by the Theragen Etex Bio Institute (Seoul, Republic of Korea, www.theragenetex.com/kr/bio). TruSeq Stranded mRNA Library Kit (Illumina) was used to build the library. Afterwards, data was processed using ‘Raw read’ for mRNA sequencing. Raw reads were aligned to GRCm38.96 (NCBI) using STAR aligner v2.7.1 for calculation of ‘RSEM’ expression values. Dobin et al., Bioinformatics 29(1): 15-21 (2013). We performed the STAR aligner as the default option. Since the total number of reads for each sample was different, normalization was performed by TMM method. Thirteen mouse samples were processed in the same way. All data is available in the GEO (Gene Expression Omnibus, https://www.ncbi.nlm.nih.gov/geo/) as GSE142633.

### Public Database Usage, Reanalysis, and Network Analysis

We used results from Weinberg et al. to confirm miRNAs that are highly related to cognitive impairment. Weinberg et al., Front Neurosci 9:430 (2015). The 100 genes shown in Figure EV1 were extracted from Table 1 of Weinberg et al. We took log 2 in Weinberg et al.'s results, ordered them, and marked the target miRNAs. The “miRDB” was used to search for miRNA targeting specific genes. Wong et al., Nucleic Acids Res 43: D146-52 (2015). The “Genecard” database was used to search for genes related to disease or biological symptoms. Rebhan et al., Bioinformatics 14(8):656-64 (1998). The results in Figure EV2A show search results from using keywords, “Inflammation”, “Amyloid beta degradation” and “Alzheimer” in August 2019. We used “VennDiagram” package of R for analysis for Venn diagram. The “GeneMAINA” (version 3.5.1) package of Cytoscape (version 3.7.1) was used for protein to protein interaction analysis. Franz et al., Nucleic Acids Res 46 (W1): W60-W64 (2018). We used 265 common genes that included hsa-miR-485-3p target genes and Alzheimer-related genes as inputs for protein interaction analysis. Among them, 139 genes interacted without neighbor gene. In addition, 9 genes were highly associated with cerebral nervous system diseases (including AD) and at the same time, low expression was reported in the patient group or in a dementia mouse model.

Intraventricular Injection of the miR-485 Inhibitor

The miR485-3p antisense oligonucleotide (ASO) (i.e., miR-485 inhibitor) (AGAGAGGAGAGCCGUGUAUGAC) (SEQ ID NO: 30) and a control oligonucleotide (“miR-control”) (CCTTCCCTGAAGGTTCCTCCTT) (SEQ ID NO: 61) were synthesized by Integrated DNA Technologies (USA). All animals were initially anesthetized with 3-5% isoflurane in oxygen and fixed on a stereotaxic frame (JeongDo). For intracerebroventricular (ICV) injection, miR-485 inhibitor or non-targeting control oligonucleotides were formulated with in vivo jetPEI reagent (Polyplus). miR-485 inhibitor (1.5 μg) or control oligonucleotide, formulated with in vivo jetPEI reagent, was injected with a 10 μL Hamilton syringe (26-gauge blunt needle) at 1.5 μL/min. The miR-485 inhibitor and the control oligonucleotides were infused in a volume of 5 μL into 10-month old 5XFAD mice by intracerebroventricular (ICV). miR-485 inhibitor or non-targeting control oligonucleotides were given once a week for 2 weeks. Intracerebroventricular (ICV) position was identified using the coordinates from the bregma: AP=−0.2 mm. L=±1.0 mm, ventral (V)=−2.5 mm.

### Glial Cell and Cortical Neuron Culture and Transfection

Mouse primary mixed glial cells were cultured from the cerebral cortices of 1- to 3-day-old C57BL/6 mice. The cerebral cortex was dissected and triturated into single-cell suspensions by pipetting. Then, single-cell suspensions were plated into 6-well plates pre-coated with 0.05 mg/ml poly-D-lysine (PDL) and cultured in DMEM medium supplemented with 25 mM glucose, 10% (vol/vol) heat-inactivated foetal bovine serum, 2 mM glutamine and 1,000 units/mL penicillin-streptomycin (P/S) for 2 weeks. Primary cortical neurons were cultured from embryonic day 17 mice. In brief, cortices were dissected and incubated in ice-cold HBSS (Welgene, LB003-02) solution and dissociated in accumax (Sigma, Cat #A7089) for 15 min at 37° C. The cultures were rinsed twice in HBSS. Mouse neurons were resuspended in neurobasal media (Gibco, Cat #21103049) containing 2% B27 (Gibco, Cat #17504), 1% sodium pyruvate, and 1% P/S. Cells were filtered through a 70 μM cell strainer (SPL, 93070), plated on culture plates and maintained at 37° C. in a humidified 5% CO2 incubator. The medium was changed every 3 days and then after 12-13 days in vitro, cells were used for experiments. Primary glial cell or cortical neurons were transfected with 100 nM miR-control, 100 nM has-miR485-3p mimic or 100 nM miR-485 inhibitor using TRANSIT-X2© Transfection Reagent (Mirus Bio).

### Luciferase Assays

Human SIRT1 3′-UTR containing the target site for miR-485-3p was amplified from cDNA by PCR amplification and inserted into the psiCHECK2 vector (Promega, Cat #C8021). HEK293T cells in a 96-well plate were co-transfected with psiCHECK2-Sirt1-3′UTR wild-type (WT) or psiCHECK2-Sirt1-3′UTR mutant (MT) and miR-485-3p using Lipofectamine 2000 (Invitrogen, Cat #11668-027). Cells were harvested 48 hours later, and the Dual Luciferase Assay System (Promega, Cat #E1910) was used to measure the luciferase reporter activities. Three independent experiment were performed in triplicate.

Human CD36 3′-UTR containing the target site for miR-485-3p was amplified from cDNA by PCR amplification and inserted into the pMir-Target vector (Addgene). HEK293T cells in 96-well plates were co-transfected with pMir-CD36-3′UTR WT or pMir-CD36-3′UTR MT and pRL-SV40 vector (Addgene) and miR-485-3p using Lipofectamine 2000 (Invitrogen, Cat #11668-027). Cells were harvested 24-48 hours later, and the Dual Luciferase Assay System (Promega, Cat #E1910) was used to measure the luciferase reporter activities. Three independent experiment were performed in triplicate.

### In Vitro Binding Assay

Streptavidin magnetic beads (Invitrogen, Cat #11205D) were prepared for in vitro binding assay as follows. Beads (50 μL) were washed five times with 500 μL of 1×B&W buffer (5 mM Tris-HCl, pH 7.4; 0.5 mM EDTA; 1 M NaCl). After removing the supernatant, beads were incubated with 500 μL of 1×B&W buffer containing 100 μg of yeast tRNA (Invitrogen, Cat #AM7119) for 2 hours at 4° C. Beads were washed twice with 500 ul of 1×B&W buffer and incubated with 200 μL of 1×B&W buffer containing 400 pmol of biotin-miR485-3p for 10 minutes at room temperature. The supernatant was removed and beads were washed twice with 500 μL of 1×B&W buffer and collected with a magnetic stand. miRNA-coated beads were incubated with 500 μL of 1× B&W buffer containing 1 μg of in vitro transcribed target mRNA overnight at 4° C. The following day, beads were washed with 1 ml of 1×B&W buffer five times and then resuspended in 200 μL of RNase-free water. Bound RNA was extracted with QiaZol Lysis reagent (Qiagen, Cat #79306) under manufacturer's instructions. Extracted RNA was quantified by StepOnePlus Real-time PCR system (Applied Biosystems, REF: 4376592).

### Western Blot

Brain tissue, primary glial cells or cortical neuron cells were homogenized in ice-cold RIPA buffer (iNtRON Biotechnology) containing protease/phosphatase inhibitor cocktail (Cell Signaling Technology, Cat #5872) on ice for 30 min. The lysates were centrifuged at 13,000 rpm for 15 min at 4° C., and supernatants were collected. The samples were separated by SDS-polyacrylamide gel electrophoresis, transferred to PVDF membranes and incubated with the following primary antibodies: rabbit anti-PGC-1α (Abcam, Cat #ab54481, 1:1000), rabbit anti-APP (Cell Signaling Technology, Cat #2452, 1:1000), mouse anti-sAPPα (IBL, Cat #11088, 1:1000), mouse anti-sAPPα (IBL, Cat #10321, 1:1000), rabbit anti-Adam10 (Abcam, Cat #ab1997, 1:100), mouse anti-CTFs (Biolegend, Cat #SIG-39152, 1:1000), rabbit anti-β-amyloid (1-42) (Cell Signaling Technology, Cat #14974, 1:1000), rabbit anti-BACE1 (Abcam, Cat #ab2077, 1:1000), mouse anti-NeuN (Millipore, #MAB377, 1:1000), rabbit anti-cleaved caspase 3 (Cell Signaling Technology, Cat #9664, 1:1000), mouse anti-GFAP (Merck, Cat #MAB360, 1:1000), rabbit anti-IL-1β (abcam, Cat #9722, 1:1000), rabbit anti-NF-kB (p65) (Cell Signaling Technology, Cat #8242, 1:1000), goat anti-Iba1 (Abcam, Cat #ab5076, 1:1000), rabbit anti-SIRT1 (Abcam, Cat #04-1557), mouse anti-TNF-α (Santa Cruz, Cat #sc-52746), anti-actin (Santa Cruz, Cat #sc-47778). The results were visualized using an enhanced chemiluminescence system, and quantified by densitometric analysis (Image J software, NIH). All experiments were performed independently at least three times.

### Soluble and Insoluble Aβ Extraction

Brain tissue samples were homogenized with RIPA buffer containing protease/phosphatase inhibitors on ice, followed by centrifugation at 12,000 rpm for 15 min. The supernatants were collected. To obtain the insoluble fraction from brain tissues, the pellet of brain lysates was lysed in insoluble extraction buffer [50 mM Tris-HCl (pH7.5)+2% SDS] containing protease/phosphatase inhibitor cocktail on ice for 30 min. The lysates were centrifuged at 4° C. for 15 min at 13,000 rpm. Protein was quantified using bicinchoninic acid (BCA) assay kit (Bio-Rad Laboratories, Cat #5000116) and adjusted to the same final concentration. After denaturation, the lysates were processed for western blotting to measure insoluble Aβ.

### Immunohistochemistry

For immunohistochemistry, miR-485 inhibitor or control oligonucleotide injected 5XFAD brains were removed, postfixed and embedded in paraffin. Coronal sections (10-μM thick) through the infarct were cut using a microtome and mounted on slides. The paraffin was removed, and the sections were washed with PBS-T and blocked in 10% bovine serum albumin for 2 hours. Thereafter, the following primary antibodies were applied: purified mouse anti-b-Amyloid, 1-16 (Biolegend, #803001, 1 μg/ml), rabbit anti-b-amyloid (1-42) (Cell Signaling Technology, #14974s, 1:100), rabbit anti-Iba-1 (Wako, #019-19741, 2 μg/ml), goat anti-Iba-1 (Abcam, #ab5076, 2 μg/ml), rabbit anti-CD68 (Abcam, #ab125212, 1 μg/ml), rabbit anti-GFAP (Abcam, #ab16997, 1:100), mouse anti-GFAP (Millipore, #MAB360, 1:500) rat anti-CD36 (Abcam, #ab80080, 1:100), mouse anti-TNF-α (Santa Cruz, #sc-52746, 1:100), rabbit anti-IL-1b (Abcam, #ab9722, 1 μg/ml), rabbit anti-cleaved caspase-3 (Cell Signaling Technology, #9662S, 1:300), mouse anti-NeuN (Millipore, #MAB377, 10 μg/ml). Images were obtained using a confocal microscope (Leica DMi8). Relative band intensity was normalized relative to actin using ImageJ software (NIH).

### Thioflavin-S Staining

For thioflavin-S (ThS) staining, the sliced brains were stained with filtered 1% aqueous Thioflavin-S solution for 8 minutes. The sections were then rinsed with 80%, 95% ethanol and three washes with distilled water. Afterward, brain slices were mounted and slides allowed to dry in the dark overnight. Images were taken on a Leica fluorescence microscope.

### Preparation of Oligomeric Aβ¹⁻⁴²

Oligomeric Aβ-42 Hexafluoroisopropanal (HFIP) peptide (#AS-64129) was obtained from AnaSpec (Fremont, Ca, USA). Aβ 1-42 oligomer (oAβ) was prepared as described previously. Coraci et al., American J of Pathology 160(1): 101-12 (2002). To form oAb synthetic human Aβ₁₋₄₂, Aβ₁₋₄₂. HFIP peptide was dissolved in DMSO to a stock concentration of 5 mM. Stocks were then diluted to 100 mM in serum free DMEM and incubated at 4° C. for 24 hours. Oligomeric Ab (oAβ) were confirmed by SDS-PAGE. In vitro phagocytosis assays (ELISA and immunocytochemistry)

BV2 microglial cells (2×10⁵) were plated in 6-well plates overnight. Cells were transfected using a TRANSIT-X2© Transfection Reagent (Mirus Bio, Cat #MIR6000) according to the manufacturer's instructions and treated with oAb for 4 hours at a final concentration of 1 mM. When applicable, anti-CD36 antibody was applied to the media with oAb. After 4 hours, media was collected from BV2 microglia. Levels of human Ab (1-42) in supernatant were measured by the human Ab42 ELISA kit (Invitrogen, Cat #KHB3441), according to the manufacturer's instructions.

In addition, glial phagocytosis was verified by fluorescence microscope. Coverslips were coated with poly-1-lysine before plating 8×10⁴ primary glial cells per coverslip resting in wells of a 24-well plate overnight. Primary glial cells were transfected using TRANSIT-X2© Transfection Reagent (Mirus Bio) according to the manufacturer's instructions and incubated in unlabeled oAb for 4 hours at a final concentration of 1 mM. After the four-hour incubation, the cells were washed with cold PBS. For Ab uptake measurement, primary glial cells were then fixed with 100% methanol for 1 hour at −20° C., washed with PBS-T and incubated at 4° C. with mouse anti-b-Amyloid 1-16, rabbit anti-GFAP (abcam, #ab16997, 1:100) and rabbit anti-Iba-1 (Wako, #019-19741, 2 μg/ml)

### FACS Analysis

All staining steps were performed in the dark and blocked with BD Fc Block. Primary glial cells were stained using the following antibodies: Alexa 488-conjugated anti-mouse CD36 (Biolegend, Cat #102607, 5 μg/ml) or isotype control Ab (Biolegend, Cat #400923, 5 μg/ml) for 30 min at 4° C. After 30 min, cells were washed with FACS buffer (PBS+1%). Data were analyzed with CellQuest (BD Bioscience) and FlowJo software (Treestar) packages.

### Real Time PCR

Total RNA was isolated using the Isolation of small and large RNA kit (Macherey Nagel, Dfiren). cDNA was synthesized using miScript II RT Kit (Qiagen, Hilden, Germany). For analysis the expression of miR-485-3p was performed by TaqMan miRNA analysis using TOPREAL™ qPCR 2× PreMIX (Enzynomics, Korea) on CFX connect system (Bio-Rad). The real-time PCR measurement of individual cDNAs was performed using SYBR green and Taq man probe to measure duplex DNA formation with the Bio-Rad real-time PCR system. Primers were as follows: Probe: FAM-CGAGGTCGACTTCCTAGA-NFQ. (SEQ ID NO: 51) miR-485-3p forward: 5′-CATACACGGCTCTCCTCTCTAAA-3′ (SEQ ID NO: 52); Mouse primer: Actin forward: 5′-TCCTGTGGCATCCATGAAAC-3′ (SEQ ID NO: 53), reverse: 5′-CAATGCCTGGGTACATGGTG-3′ (SEQ ID NO: 54); TNF forward: 5′-CCAAGTGGAGGAGCAGCT-3′ (SEQ ID NO: 55), reverse: 5′-GACAAGGTACAACCCATCGG-3′ (SEQ ID NO: 56); IL-1β forward: 5′-TTCGACACATGGGATAACGAGG-3′ (SEQ ID NO: 57), reverse: 5′-TTTTTGCTGTGAGTCCCGGAG-3′ (SEQ ID NO: 58); miR-16 forward: 5′-CAGCCTAGCAGCACGTAAAT-3′ (SEQ ID NO: 59); reverse: 5′-GAATCGAGCACCAGTTACG-3′ (SEQ ID NO: 60); miR-16 level was used for normalization. The relative gene expression was analyzed by the 2-ΔΔct method.

### Behavior Tests (Y-Maze and Passive Avoidance)

The Y-maze consisted of three black, opaque, plastic arms (30 cm×8 cm×15 cm) 120° from each other. The 5XFAD mice were placed in the center and were allowed to explore all three arms. The number of arm entries and number of trials (a shift is 10 cm from the center, entries into three separate arms) were recorded to calculate the percentage of alternation. An entry was defined as all three appendages entering a Y-maze arm. Alternation behavior was defined as the number of triads divided by the number of arm entries minus 2 and multiplied by 100. The passive avoidance chamber was divided into a white (light) and a black (dark) compartment (41 cm×21 cm×30 cm). The light compartment contained a 60 W electric lamp. The floor (of the dark) department contained a number of (2-mm) stainless steel rods spaced 5 mm apart. The test was done for 3 days. The first day adapts the mouse for 5 minutes in a bright zone. The second day is the training phase. The study consists of two steps. The first step places each mouse in the light zone which is then moved to the dark zone twice. One hour after the first step, each mouse is placed in the light compartment. The door separating the two compartments was opened 30 seconds later and after mice enter the dark compartment, the door was closed and an electrical foot shock (0.3 mA/10 g) was delivered through the grid floor for 3 seconds. If the mouse does not go into the dark zone for more than 5 minutes, it is considered to have learned avoidance, and the training was done up to 5 times. Twenty-four hours after the training trial, mice were placed in the light chamber for testing. Latency was defined as the time it took for a mouse to enter the dark chamber after the door separating the two compartments opened. The time taken for the mouse to enter the dark zone and exit to the bright zone was defined as TDC (time spent in the dark compartment).

### Data Analysis

All data are presented as the mean±SD. NGS data were analyzed using R (version 3.5.2). Statistical significance in the values obtained for two different groups were determined using unpaired t-test. Statistical tests were performed using GraphPad Prism 5 (GraphPad Software, La Jolla, Calif.). Behavior tests were assessed by nonparametric statistical procedures.

### Example 2: Preparation of miR-485 Inhibitor

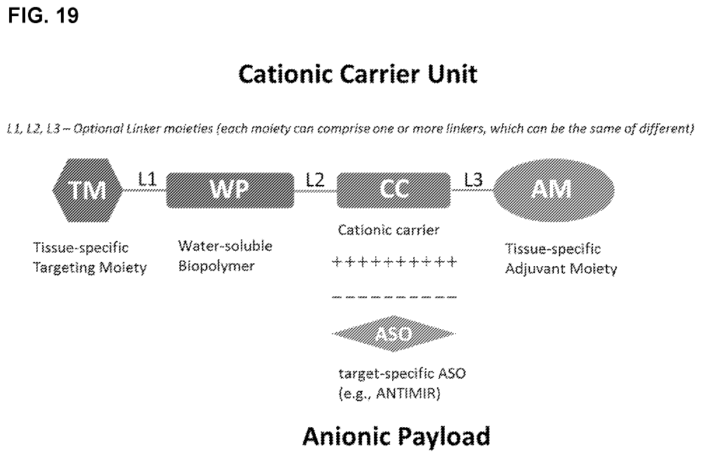

(a) Synthesis of alkyne modified tyrosine: An alkyne modified tyrosine was generated as an intermediate for the synthesis of a tissue specific targeting moiety (TM, see FIG. 19) of a cationic carrier unit to direct micelles of the present disclosure to the LAT1 transporter in the BBB.

A mixture of N-(tert-butoxycarbonyl)-L-tyrosine methyl ester (Boc-Tyr-OMe) (0.5 g, 1.69 mmol) and K₂CO₃ (1.5 equiv., 2.54 mmol) in acetonitrile (4.0 ml) was added drop by drop to propargyl bromide (1.2 equiv., 2.03 mmol). The reaction mixture was heated at 60° C. overnight. After the reaction, the reaction mixture was extracted using water:ethyl acetate (EA). Then, the organic layer was washed using a brine solution. The crude material was purified by flash column (EA in hexane 10%). Next, the resulting product was dissolved in 1,4-dioxane (1.0 ml) and 6.0 M HCl (1.0 ml). The reaction mixture was heated at 100° C. overnight. Next, the dioxane was removed and extracted by EA. Aqueous NaOH (0.5 M) solution was added to the mixture until the pH value become 7. The reactant was concentrated by evaporator and centrifuged at 12,000 rpm at 0° C. The precipitate was washed with deionized water and lyophilized.

(b) Synthesis of poly(ethylene glycol)-b-poly(L-lysine) (PEG-PLL): This synthesis step generated the water-soluble biopolymer (WP) and cationic carrier (CC) of a cationic carrier unit of the present disclosure (see FIG. 19).

Poly(ethylene glycol)-b-poly(L-lysine) was synthesized by ring opening polymerization of Lys(TFA)-NCA with monomethoxy PEG (MeO-PEG) as a macroinitiator. In brief, MeO-PEG (600 mg, 0.12 mmol) and Lys(TFA)-NCA (2574 mg, 9.6 mmol) were separately dissolved in DMF containing 1M thiourea and DMF (or NMP). Lys(TFA)-NCA solution was dropped into the MeO-PEG solution by micro syringe and the reaction mixture was stirred at 37° C. for 4 days. The reaction bottles were purged with argon and vacuum. All reactions were conducted in argon atmosphere. After the reaction, the mixture was precipitated into an excess amount of diethyl ether. The precipitate was re-dissolved in methanol and precipitated again into cold diethyl ether. Then it was filtered and white powder was obtained after drying in vacuo. For the deprotection of TFA group in PEG-PLL (TFA), the next step was followed.

MeO-PEG-PLL (TFA) (500 mg) was dissolved in methanol (60 mL) and 1N NaOH (6 mL) was dropped into the polymer solution with stirring. The mixture was maintained for 1 day with stirring at 37° C. The reaction mixture was dialyzed against 10 mM HEPES for 4 times and distilled water. White powder of PEG-PLL was obtained after lyophilization.

(b) Synthesis of azido-poly(ethylene glycol)-b-poly(L-lysine) (N₃-PEG-PLL): This synthesis step generated the water-soluble biopolymer (WP) and cationic carrier (CC) of a cationic carrier unit of the present disclosure (see FIG. 19).

Azido-poly(ethylene glycol)-b-poly(L-lysine) was synthesized by ring opening polymerization of Lys(TFA)-NCA with azido-PEG (N₃—PEG). In brief, N₃—PEG (300 mg, 0.06 mmol) and Lys(TFA)-NCA (1287 mg, 4.8 mmol) were separately dissolved in DMF containing 1M thiourea and DMF (or NMP). Lys(TFA)-NCA solution was dropped into the N₃-PEG solution by micro syringe and the reaction mixture was stirred at 37° C. for 4 days. The reaction bottles were purged with argon and vacuum. All reactions were conducted in argon atmosphere. After the reaction, the mixture was precipitated into an excess amount of diethyl ether. The precipitate was re-dissolved in methanol and precipitated again into cold diethyl ether. Then it was filtered and white powder was obtained after drying in vacuo. For the deprotection of TFA group in PEG-PLL (TFA), the next step was followed.

N₃-PEG-PLL (500 mg) was dissolved in methanol (60 mL) and 1N NaOH (6 mL) was dropped into the polymer solution with stirring. The mixture was maintained for 1 day with stirring at 37° C. The reaction mixture was dialyzed against 10 mM HEPES for 4 times and distilled water. White powder of N₃-PEG-PLL was obtained after lyophilization.

(c) Synthesis of (methoxy or) azido-poly(ethylene glycol)-b-poly(L-lysine/nicotinamide/mercaptopropanamide) (N₃-PEG-PLL(Nic/SH)): In this step, the tissue-specific adjuvant moieties (AM, see FIG. 19) were attached to the WP-CC component of a cationic carrier unit of the present disclosure. The tissue-specific adjuvant moiety (AM) used in the cationic carrier unit was nicotinamide (vitamin B3). This step would yield the WP-CC-AM components of the cationic carrier unit depicted in FIG. 19.

Azido-poly(ethylene glycol)-b-poly(L-lysine/nicotinamide/mercaptopropanamide) (N₃-PEG-PLL(Nic/SH)) was synthesized by chemical modification of N₃-PEG-PLL and nicotinic acid in the presence of EDC/NHS. N₃—PEG-PLL (372 mg, 25.8 μmol) and nicotinic acid (556.7 mg, 1.02 equiv. to NH2 of PEG-PLL) were separately dissolved in mixture of deionized water and methanol (1:1). EDC.HCl (556.7 mg, 1.5 equiv. to NH₂ of N₃-PEG-PLL) was added into nicotinic acid solution and NHS (334.2 mg, 1.5 equiv. to NH2 of PEG-PLL) stepwise added into the mixture.

The reaction mixture was added into the N₃-PEG-PLL solution. The reaction mixture was maintained at 37° C. for 16 hours with stirring. After 16 hours, 3,3′-dithiodiproponic acid (36.8 mg, 0.1 equiv.) was dissolved in methanol, EDC.HCl (40.3 mg, 0.15 equiv.), and NHS (24.2 mg, 0.15 equiv.) were dissolved each in deionized water. Then, NHS and EDC.HCl were added sequentially into 3,3′-dithiodiproponic acid solution. The mixture solution was stirred for 4 hours at 37° C. after adding crude N₃-PEG-PLL(Nic) solution.

For purification, the mixture was dialyzed against methanol for 2 hours, added DL-dithiothreitol (DTT, 40.6 mg, 0.15 equiv.), then activated for 30 min.

For removing the DTT, the mixture was dialyzed sequentially methanol, 50% methanol in deionized water, deionized water.

d) Synthesis of Phenyl alanine-poly(ethylene glycol)-b-poly(L-lysine/nicotinamide/mercaptopropanamide) (Phe-PEG-PLL(Nic/SH)): In this step, the tissue-specific targeting moiety (TM) was attached to the WP-CC-AM component synthesized in the previous step. The TM component (phenyl alanine) was generated by reaction of the intermediate generated in step (a) with the product of step (c).

To target brain endothelial tissue in blood vessels, as a LAT1 targeting amino acid, phenyl alanine was introduced by click reaction between N₃-PEG-PLL(Nic/SH) and alkyne modified tyrosine in the presence of copper catalyst In brief, N₃—PEG-PLL(Nic/SH) (130 mg, 6.5 μmol) and alkyne modified phenyl alanine (5.7 mg, 4.0 equiv.) were dissolved in deionized water (or 50 mM sodium phosphate buffer). Then, CuSO4.H2O (0.4 mg, 25 mol %) and Tris(3-hydroxypropyltriazolylmethyl)amine (THPTA, 3.4 mg, 1.2 equiv.) were dissolved deionized water and added N₃-PEG-PLL(Nic/SH) solution. Then, sodium ascorbate (3.2 mg, 2.5 equiv.) were added into the mixture solution. The reaction mixture was maintained with stirring for 16 hours at room temperature. After the reaction, the mixture was transferred into dialysis membranes (MWCO=7,000) and dialyzed against deionized water for 1 day. The final product was obtained after lyophilization.

(e) Polyion Complex (PIC) micelle preparation—Once the cationic carrier units of the present disclosure were generated as described above, micelles were produced. The micelles described in the present example comprised cationic carrier units combined with an antisense oligonucleotide payload.

Nano sized PIC micelles were prepared by mixing MeO- or Phe-PEG-PLL(Nic) and miRNA. PEG-PLL(Nic) was dissolved in HEPES buffer (10 mM) at 0.5 mg/mL concentration. Then a miRNA solution (22.5 μM) in RNAse free water was mixed with the polymer solution at 2:1 (v/v) ratio of miRNA inhibitor (SEQ ID NOs: 2-30) (e.g., AGAGAGGAGAGCCGUGUAUGAC; SEQ ID NO: 30) to polymer.

The mixing ratio of polymer to anti-miRNA was determined by optimizing micelle forming conditions, i.e., ratio between amine in polymer (carrier of the present disclosure) to phosphate in anti-miRNA (payload). The mixture of polymer (carrier) and anti-miRNA (payload) was vigorously mixed for 90 seconds by multi-vortex at 3000 rpm, and kept at room temperature for 30 min to stabilize the micelles.

Micelles (10 μM of Anti-miRNA concentration) were stored at 4° C. prior to use. MeO- or Phe-micelles were prepared using the same method, and different amounts of Phe-containing micelles (25%˜75%) were also prepared by mixing both polymers during micelle preparation.

### Example 3: Analysis of SIRT1 Expression in Alzheimer's Disease

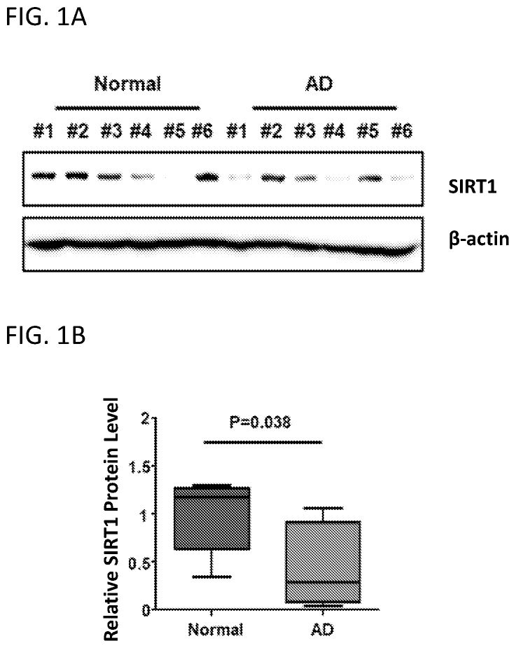

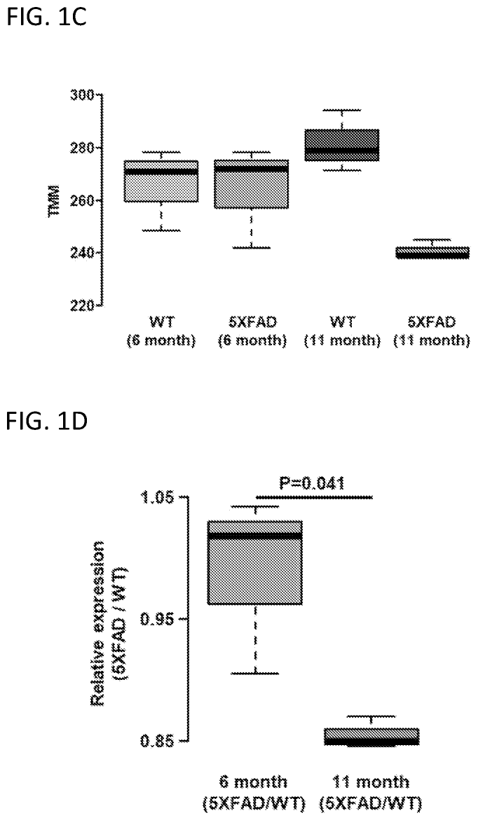

Previous studies reported that SIRT1 levels were reduced in brains of human AD patients and this reduction affected AD progression from early to late stages (Julien et al, 2009, Lutz et al, 2014). To begin assessing the potential therapeutic effects of miR-485 inhibitors disclosed herein, SIRT1 expression was assessed in postmortem brain (precentral gyrus) samples from Alzheimer's disease (AD) patients. As shown in FIGS. 1A and 1B, SIRT1 protein levels were notably reduced in AD patient brains compared to normal human brains.

To confirm the above results, SIRT1 expression was assessed in an established AD animal model (i.e., five familial AD mutation (5XFAD) transgenic mice). As shown in 1C, there was no significant difference in SIRT1 expression between the 6-month old AD mice compared to the wild-type control animals. However, in the 11-month old AD mice, there was a significant reduction in SIRT1 expression (see FIG. 1C). SIRT1 expression was gradually reduced as the 5XFAD aged mice (FIG. 1D).

The above results confirm the earlier studies and demonstrate that SIRT1 expression is down-regulated in AD, suggesting that SIRT1 can play a role in AD pathogenesis.

### Example 4: Analysis of the Potency of miR-485 Inhibitors in Regulating miR485-3p Expression

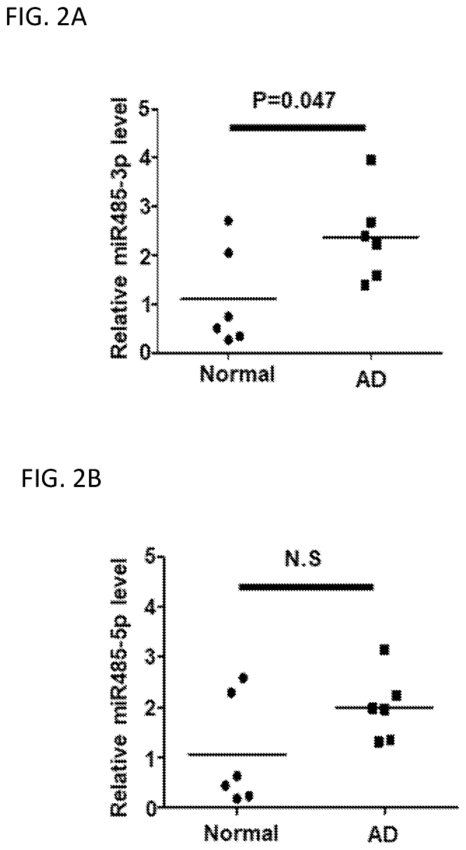

To identify potential miRNA candidates that could regulate SIRT1 expression, brain samples of AD patients were further analyzed for miRNAs that were overexpressed in the samples. As shown in FIG. 2A, miR485-3p expression was significantly higher in precentral gyrus tissue of AD patients compared to normal healthy tissue. No significant differences were observed for other SIRT1 related miRNAs, including miR485-5p (see FIG. 2B).

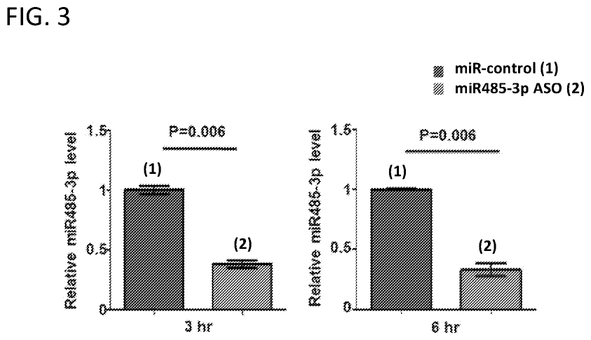

Next, using publicly available algorithms (e.g., Targetscan and miRbase programs), it was predicted that miR-485-3p has a binding site in the 3′UTR of SIRT1. To confirm, the ability of human miR485-3p mimic and inhibitor to regulate miR485-3p expression in mice was assessed. As shown in FIG. 3, using real time PCR analysis, a significant reduction in miR485-3p expression was observed in mouse primary cortical neurons when transfected with a human miR485-3p inhibitor.

This result confirms that the miR485 inhibitors disclosed herein can reduce and/or inhibit miR485-3p expression.

### Example 5: Analysis of miR-485 Inhibitor Regulation of SIRT1

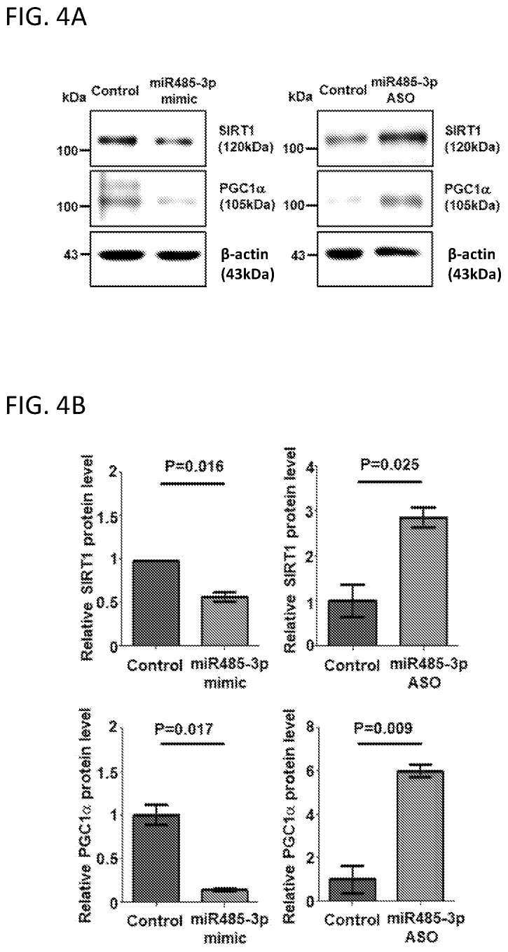

To understand the relationship between miR485-3p and SIRT1 expression, mouse primary cortical neurons were transfected with one of the following: (i) human miR-control, (ii) human miR485-3p, or (iii) miR485-3p inhibitor. Then, the expression of SIRT1 was assessed in the transfected cells. As shown in FIGS. 4A and 4B, SIRT1 protein expression was reduced in miR485-3p transfected primary cortical neurons compared to miR-control transfected neurons. In contrast, primary cortical neurons transfected with the miRNA inhibitor disclosed herein expressed significantly higher level of SIRT1 protein. And, as shown in FIGS. 3A and 3B, SIRT1 expression appeared to be correlated with PGC-1α expression.

These results demonstrate that the miR485 inhibitors of the present disclosure can increase SIRT1 expression by regulating miR485-3p expression. The results further demonstrate that the miRNA inhibitors disclosed herein can also be useful in increasing PGC-1α protein expression in cells.

### Example 6: Analysis of the Binding of miR485-3p to SIRT1

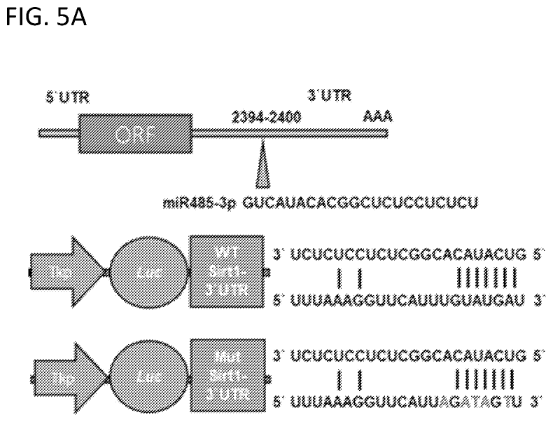

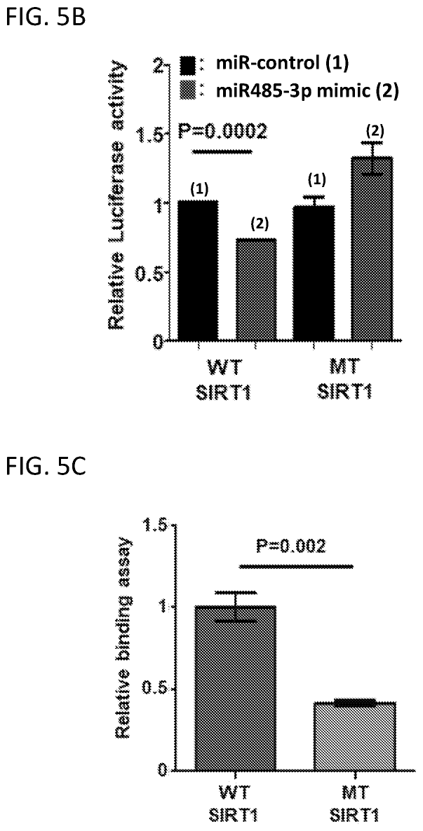

To confirm the target site for miR485-3p on SIRT1, luciferase reporter plasmids of the SIRT1 3′-UTR containing either wild-type or mutated sequence of the potential miR485-3p site were constructed (see FIG. 5A). Then, HEK293T cells were transfected with the plasmids, and promoter activity was measured in the transfected cells. As shown in FIG. 5B, wild type promoter activity was significantly reduced but the mutant form was not different in miR485-3p transfected cells.

Next, the physical binding of miR485-3p to the 3′ UTR of SIRT1 was assessed using an in vitro binding assay. Briefly, streptavidin-miR485-3p-coated magnetic beads were incubated with in vitro transcribed wild type 3′ UTR and mutant 3′ UTR of SIRT1 respectively. Binding RNA was eluted and quantified by real-time PCR. Relative binding was calculated using the formula: Relative Binding=100×2^{((Adjusted input)-(Ct IP))}/100×2^{((Adjusted input)-(Ct WT))} Compared to wild-type seed (i.e., site where miRNA binds) sequences, the relative binding efficiency was significantly reduced in 3′ UTR containing mutant seed sequences (see FIG. 5C).

The above results collectively demonstrate that miR485-3p can directly target the 3′-UTR of SIRT1 and that this interaction can negatively regulate SIRT1 expression.

### Example 7: Analysis of miR-485 Inhibitor on Beta Amyloid (Aβ) Plaque Formation

To explore the potential therapeutic benefits of miR-485 inhibitors disclosed herein on Alzheimer's disease, the effect of miR-485 inhibitors on amyloid plaque formation and insoluble Aβ levels was assessed in 10-month old 5XFAD mice. It has been shown that 5XFAD transgenic mice exhibit show amyloid plaque deposition starting at 2 months and that the aggregation of Aβ into such plaques worsens as AD progresses. Eimer et al., Mol Neurodegener 8:2 (2013); and Näslund et al., Proc Natl Acad Sci 91:8378-08382 (1994).

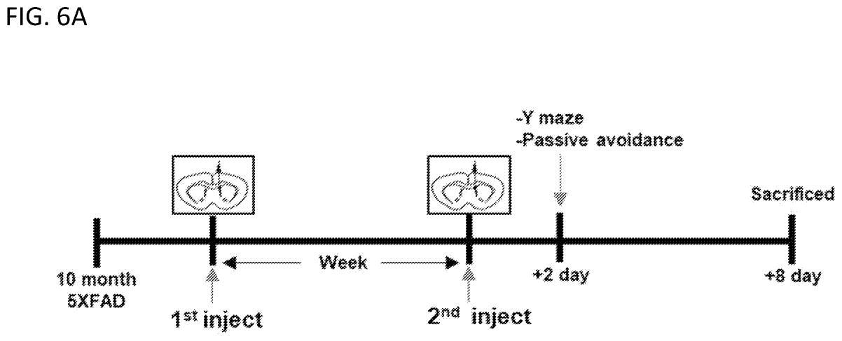

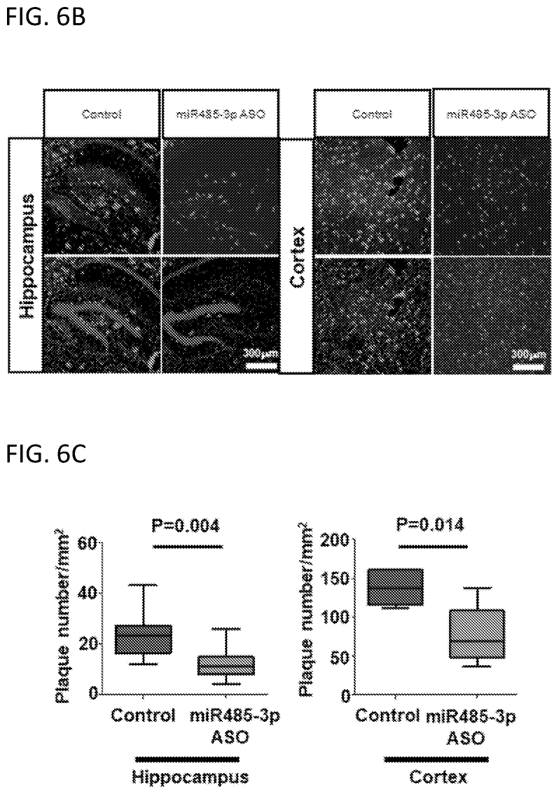

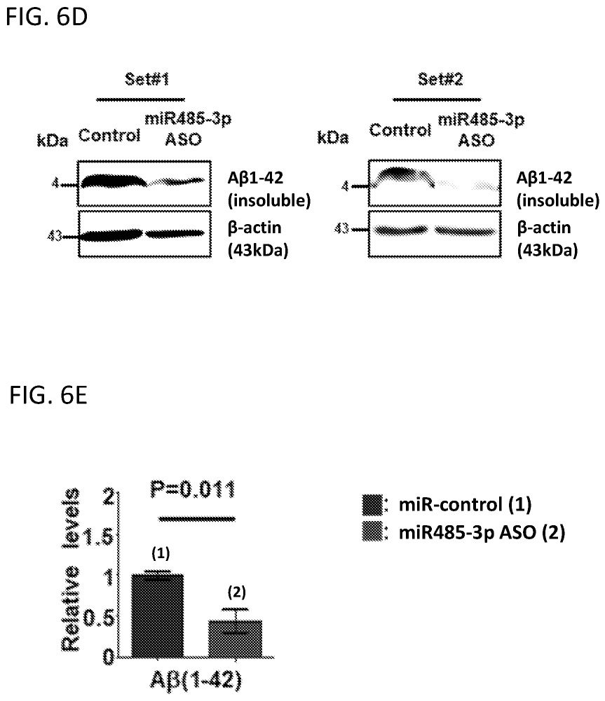

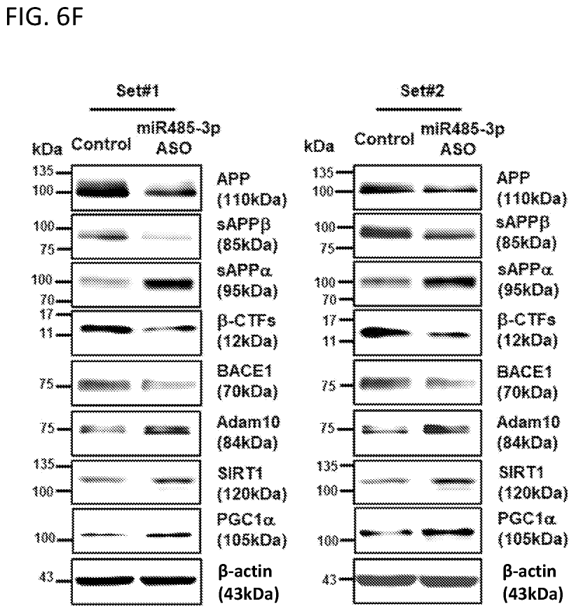

Briefly, miR-485 inhibitor formulated with in vivo jetPEI reagent was injected in the right lateral ventricle of the animals by stereotaxic injection. The animals received a second administration a week later (see FIG. 6A). Then, the number of amyloid plaque formation was quantified using immunofluorescence microscopy using 6E10 staining and thioflavin S. As shown in FIGS. 6B and 6C, the number of amyloid plaques was markedly decreased in 5XFAD animals treated with the miR-485 inhibitor compared to the animals treated with the miR-control, suggesting that the miR-485 inhibitor can ameliorate amyloid burden in AD mice.

To further investigate the effect of miR-485 inhibitors on Aβ production, the levels of insoluble Aβ1-42, amyloid precursor protein (APP) and APP processing enzymes, including α-secretase, ADAM (A disintegrin and metalloprotease) 10, and β-secretase BACE1 were assessed in the frontal cortex of the AD mice from the different treatment groups.

As shown in FIGS. 6D and 6E, there was a significant reduction in insoluble Aβ1-42 production in AD mice treated with the miR-485 inhibitor compared to the control animals (i.e., treated with miR-control). miR-485 treated animals also exhibited decreased levels of β-CTFs and sAPPβ (i.e., the main products of BACE) in the frontal cortex, compared to the control animals (see FIGS. 6F and 6G). Accordingly, there was also reduced expression of BACE1 in the inhibitor treated AD animals. And, confirming the results shown earlier (see Example 3), AD mice treated with the miR-485 inhibitor had significantly reduced levels of SIRT1 and PGC-1α protein. However, some of the proteins tested were not negatively regulated by miR-485 administration. For instance, there was no significant difference in total APP levels among the animals from the different treatment groups (see FIGS. 6F and 6G). And, for Adam10 and sAPPα proteins, administration of the miR-485 inhibitor significantly increased the expression of these proteins compared to the control animals.

The above results demonstrate that the miR-485 inhibitors disclosed herein can regulate different genes and thereby, reduce both Aβ production and plaque formation in vivo.

### Example 8: Analysis of miR-485 Inhibitor on AP Plaque Phagocytosis

Alzheimer's disease is caused by imbalances between Aβ production and clearance. Previous studies have shown that glial cells mediate clearance and phagocytosis of aggregated Aβ in AD brain, where they contribute to the alleviation of AD. Ries et al., Front Aging Neurosci 8:160 (2016). Therefore, to further explore the role of glial cells in AD, the colocalization of glial cells and Aβ plaque was assessed in AD mice using immunohistochemistry analysis using Iba1 and 6E10 antibodies.

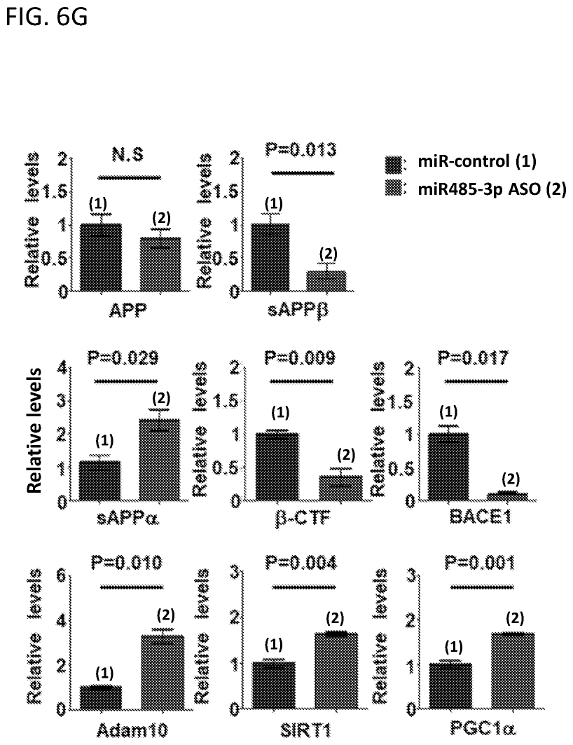

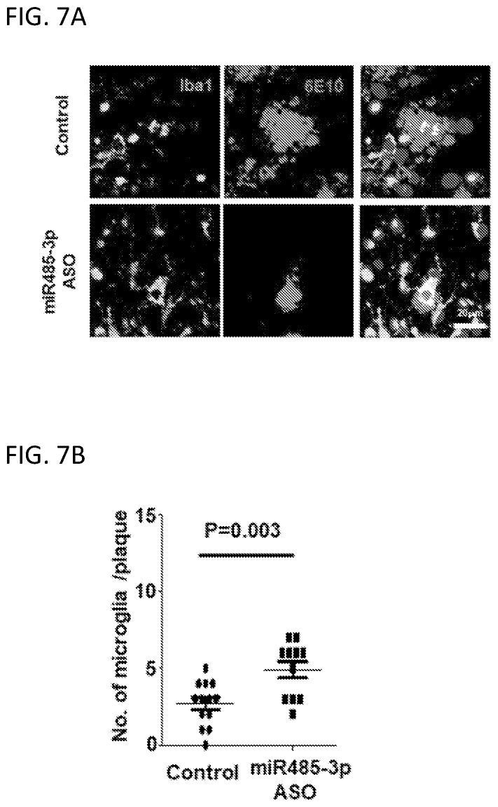

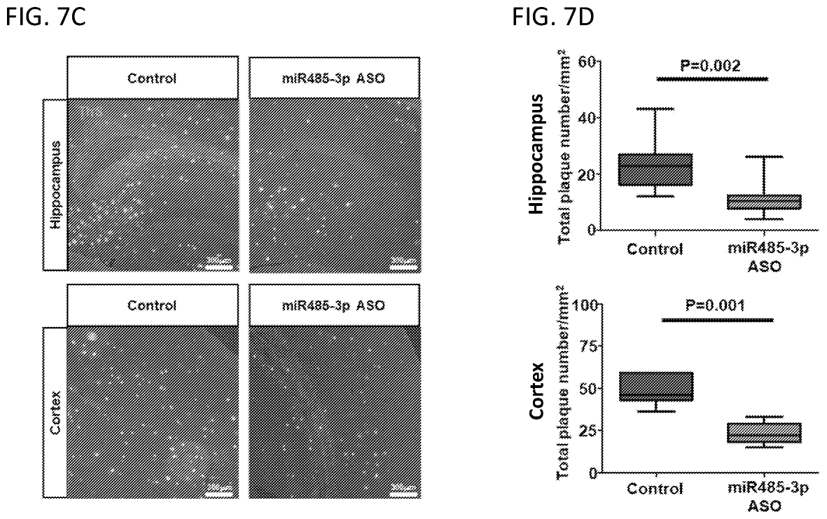

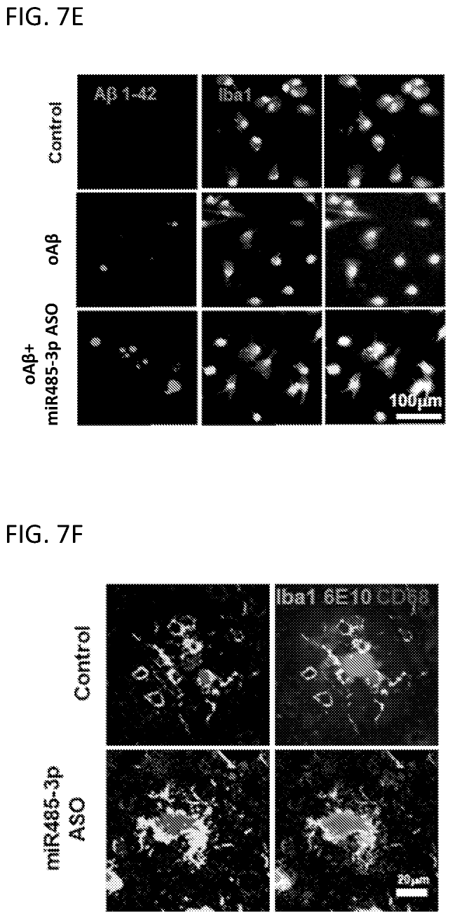

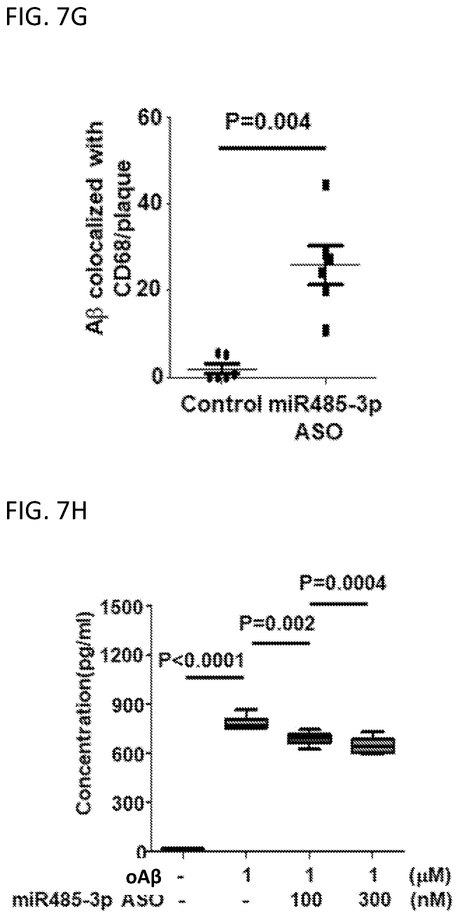

As shown in FIGS. 7A-7D, there was significantly higher colocalization of Aβ plaque and glial cells in AD mice treated with miR-485 inhibitor. In addition, administration of the miR-485 inhibitor to the AD mice consistently increased the uptake of Aβ plaques by the primary glial cells (see FIG. 7E).

Next, to further assess Aβ engulfment and clearance by glial cell, the number of CD68+ microglial phagosomes that had internalized Aβ plaques was quantified using CD68, 6E10, and Iba1 coimmunostaining. CD68, a transmembrane glycoprotein of the lysosome/endosome-associated membrane glycoprotein family, acts as a scavenger receptor for debris clearance. Yamada et al., Cell Mol Life Sci 54(7):628-40 (1998).

As shown in FIGS. 7F and 7G, the clustering of Iba1+ microglia surrounding amyloid plaques exhibited a diffuse CD68 distribution in AD mice treated with the miR-485 inhibitor, compared to the control animals (i.e., treated with miR-control).

To confirm the above results, Aβ aggregates were prepared by incubating Aβ monomers (100 μM) at 4° C. overnight then diluting the peptide stock with cell culture medium. Then, primary glial cells were transfected with the miR-485 inhibitor and further treated with 1 mM oligomeric amyloid beta (oAb) for 3 hours. Consistent with the above results, Aβ levels in conditioned media were considerably reduced in miR485-3p ASO transfected cells compare to control transfected cells (FIG. 7H).

The above results demonstrate that the miR-485 inhibitors disclosed herein can enhance microglial Aβ phagocytosis.

### Example 9: Analysis of miR-485 Inhibitor Regulation of CD36

As described herein, CD36/SR-BII can contribute to the phagocytosis of Aβ by glial cells. Using publicly available algorithms (see Example 2), it was predicted that miR-485-3p also has a binding site in the 3′UTR of CD36. Accordingly, to assess whether the miR-485 inhibitors disclosed herein can also regulate CD36 expression, AD mice were treated with either a miR-485 inhibitor or miR-control (as described in the earlier examples), and then the expression of CD36 was assessed in the animals.

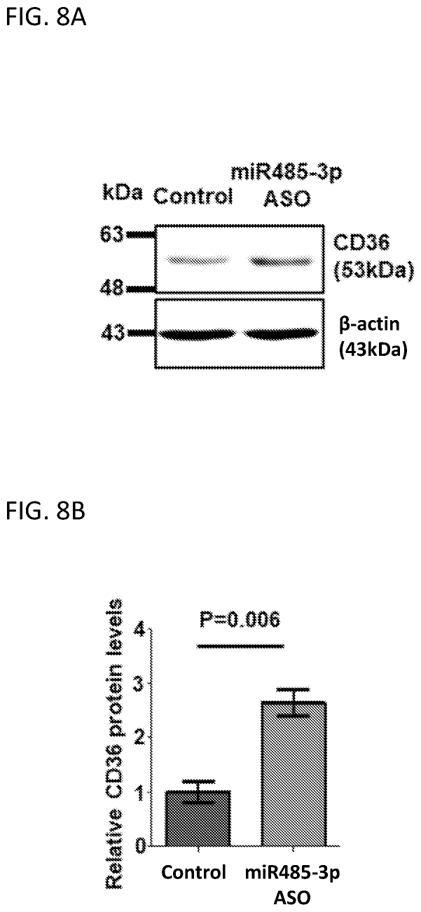

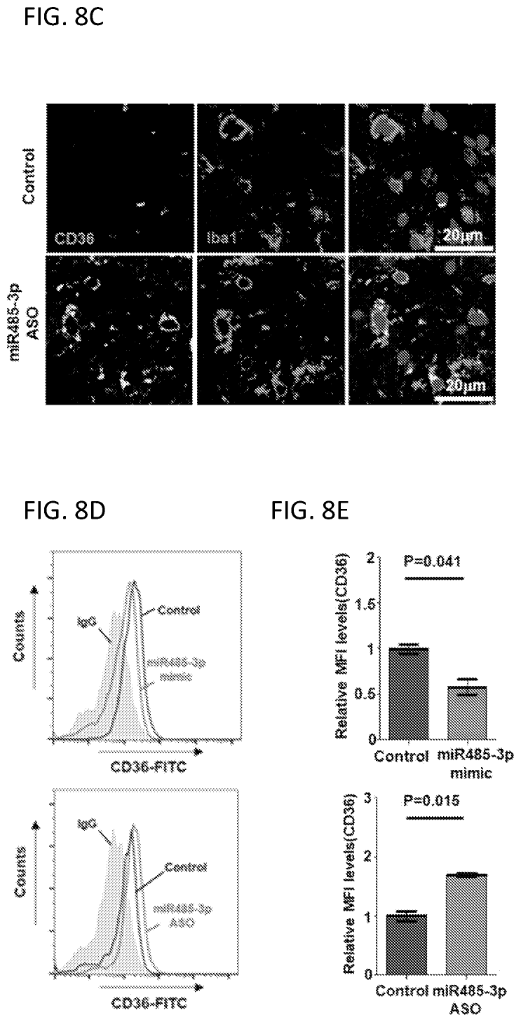

As shown in FIGS. 8A and 8B, AD mice treated with the miR-485 inhibitor exhibited significantly higher CD36 expression compared to the control animals. In addition, CD36 expression was noticeably higher in Iba-1-positive microglial cells using immunohistochemistry (FIG. 8C).

Based on the above observations, it was next examined whether transfection with miR485-3p or miR-485 inhibitor could alter CD36 expression in mouse primary glial cells. As shown in FIGS. 8D and 8E, CD36 expression was markedly decreased in miR485-3p transfected primary glial cell compared to miR-controls. In contrast, cells transfected with the miR-485 inhibitor exhibited significantly higher CD36 expression.

The above results demonstrate that the miR-485 inhibitors of the present disclosure can also increase CD36 expression by regulating the expression of miR-485-3p.

### Example 10: Analysis of the Binding of miR485-3p to CD36

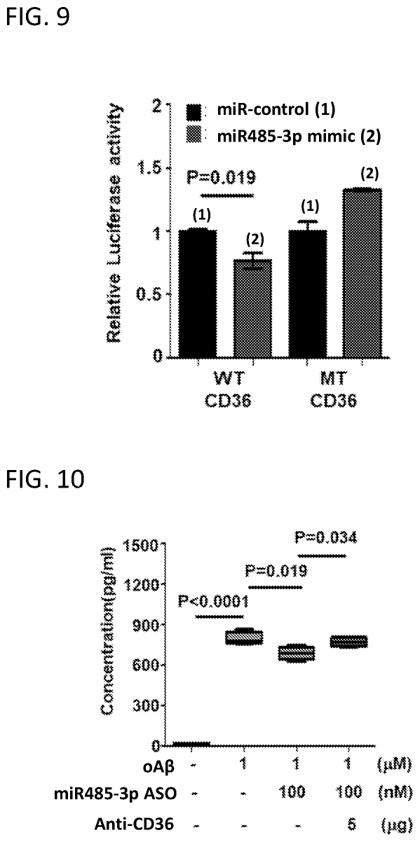

To confirm the target site for miR485-3p within the 3′-UTR of CD36, luciferase reporter plasmids containing either wild-type or mutated sequence of the potential miR485-3p site were constructed. Then, HEK293T cells were transfected with the plasmids, and promoter activity was measured in the transfected cells. As shown in FIG. 9, wild type promoter activity was significantly reduced but the mutant form was not different in miR485-3p transfected cells.

Next, the physical binding of miR485-3p to the 3′ UTR of SIRT1 was assessed using an in vitro binding assay as described in Example 4. The relative binding efficiency was significantly reduced in 3′ UTR-containing mutant seed sequences.

The above results collectively demonstrate that miR485-3p can directly target the 3′-UTR of CD36 and that this interaction can negatively regulate CD36 expression.

### Example 11: Analysis of CD36 Regulation on AP Phagocytosis

To further assess the role of CD36+ glial cells on Aβ phagocytosis, it was examined whether a CD36 inhibitory antibody can influence glial phagocytosis. Briefly, primary glial cells were transfected with either the miR-485 inhibitor or miR-control. The transfected cells were treated with either CD36 blocking antibody or control IgG, and then treated with 1 μM oligomeric amyloid beta (oAβ) for 3 hours. An ELISA assay was used to determine Aβ phagocytosis in the conditioned media collected from the different transfected cells.

As shown in FIG. 10, Aβ levels were considerably decreased in cells transfected with the miR-485 inhibitor compared to the control transfected cells. However, this effect was significantly abrogated in cells treated with the CD36 blocking antibody.

These results confirm that the miR-485 inhibitors disclosed herein can regulate CD36 expression in a miR485-3p dependent manner, and can thereby, affect Aβ phagocytosis.

### Example 12: Analysis of miR-485 Inhibitor on Neuroinflammation

AD is known to be associated with inflammation within the brain, and the secretion of inflammatory mediators by Aβ-stimulated-glia can contribute to neuronal loss and cognitive decline. Cunningham et al., J Neurosci 25(40):9275-84 (2005). Therefore, to assess whether the miR-485 inhibitors disclosed herein has any effect on neuroinflammation, primary glial cells were transfected with the miR-485 inhibitor or miR-control, and subsequently treated with 1 μM oligomeric amyloid beta (oAβ). Then, the levels of SIRT1 and different inflammatory mediators (i.e., NF-κB, TNF-α, and IL-1β) were examined in the cells.

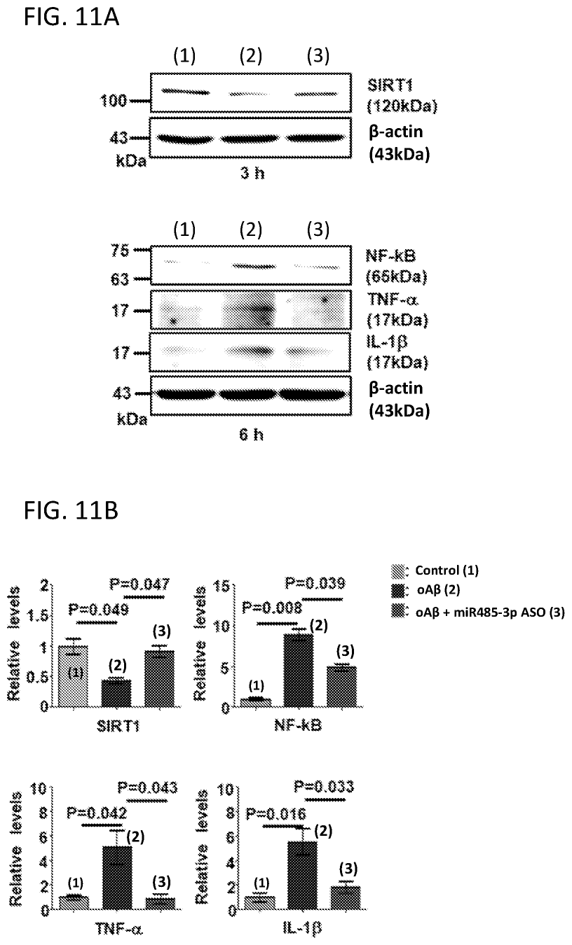

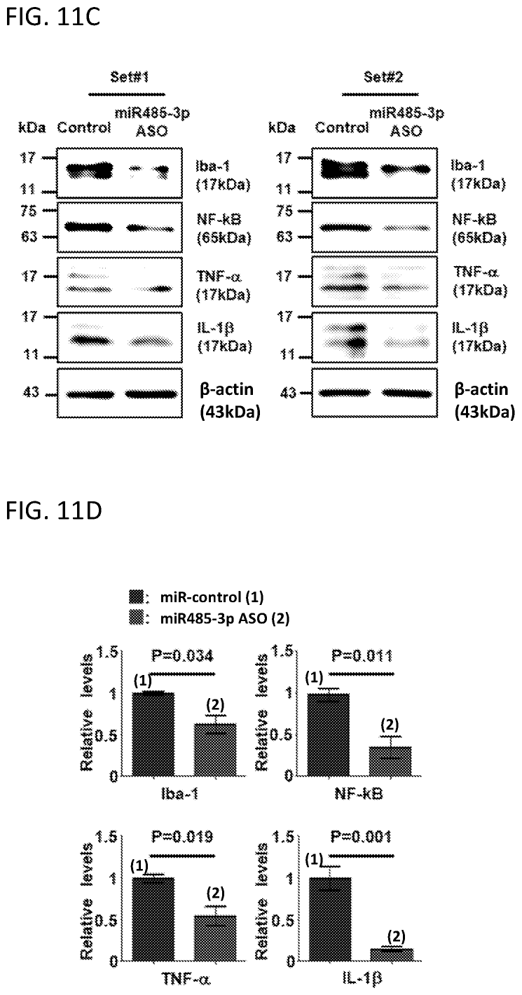

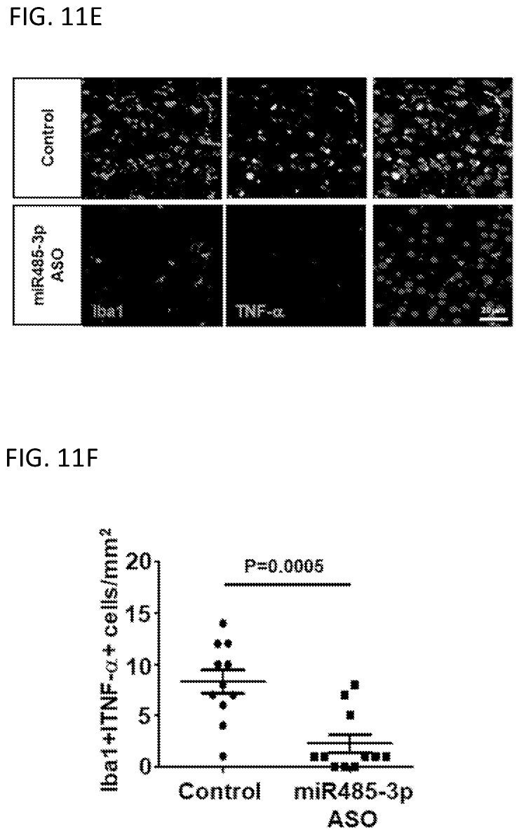

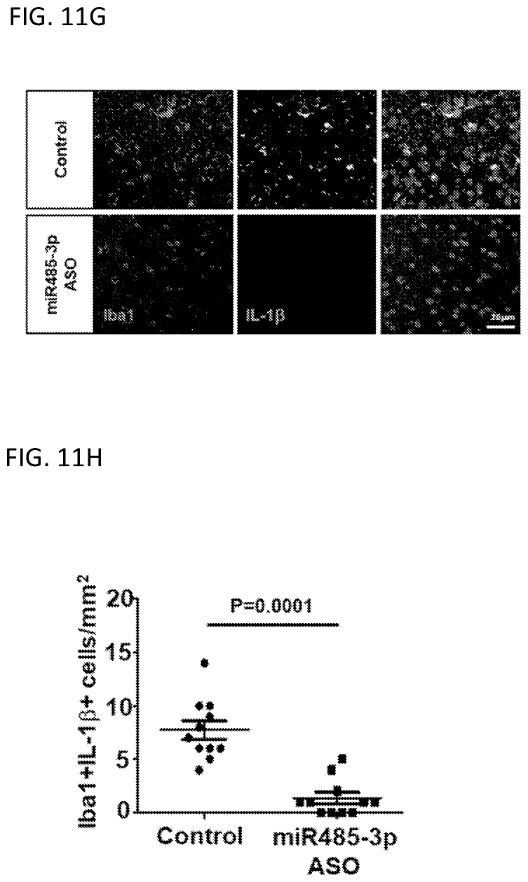

As shown in FIGS. 11A and 11B (and in agreement with the earlier data—see Example 3), SIRT1 expression was markedly decreased in oAβ treated primary glial cells, but this reduction was significantly recovered in cells transfected with the miR-485 inhibitor. The observed SIRT1 expression correlated with NF-κB expression, as well as expression levels of TNF-α and IL-1β (see FIGS. 11A and 11B). In cells transfected with the miR-485 inhibitor, there was significantly reduced levels of these inflammatory mediators.

To further characterize the effect of miR-485 inhibitor on neuroinflammation, AD mice were treated with the miR-485 inhibitor as described earlier (see Example 1). Then, the expression pattern of Iba-1 (i.e., activated microglial marker) and GFAP (i.e., activated astrocyte marker) was assessed.

As shown in FIGS. 11C and 11D, microglia expressing high levels of Iba-1 and astrocytes expressing high levels of GFAP were significantly decreased in AD mice treated with the miR-485 inhibitor. And, as observed above with the transfected cells, expression levels of NF-κB, TNF-α, and IL-1β were also significantly lower in the miR-485 inhibitor treated animals, as measured using real time PCR, Western blot, and immunohistochemistry (see FIGS. 11E-11H).

The above results demonstrate that by reducing miR485-3p expression, the miR-485 inhibitors disclosed herein can affect glial cell activation and reduce proinflammatory cytokine production via regulation SIRT1/NF-κB signaling.

### Example 13: Analysis of miR-485 Inhibitor on Neuronal Loss and Post-Synapse

As described earlier, 5XFAD transgenic mice exhibit amyloid plaque deposition starting at 2 months and neuronal loss in cortical layer V at 9 months (see Example 6). Synaptic and neuronal loss in 5XFAD mice have been correlated with Aβ accumulation and neuroinflammation. Eimer et al., Mol Neurodegener 8:2 (2013). In light of the results from the earlier examples (e.g., that the regulation of SIRT1 and CD36 expression with an miR-485 inhibitor can control Aβ processing, phagocytosis, and inflammation in AD mice), whether the miR-485 inhibitors disclosed herein have any effect on neuronal cell death was examined by assessing NeuN (a neuronal cell marker) and cleaved caspase-3.

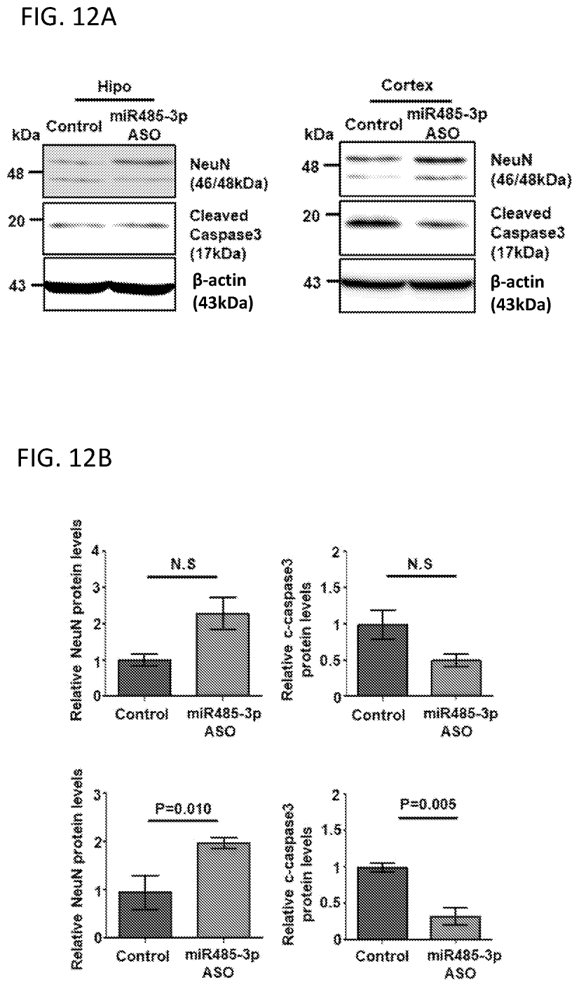

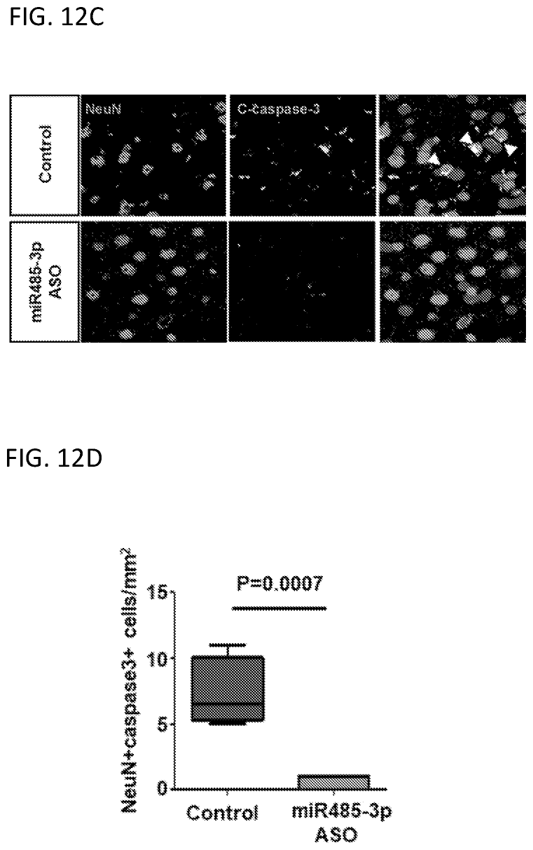

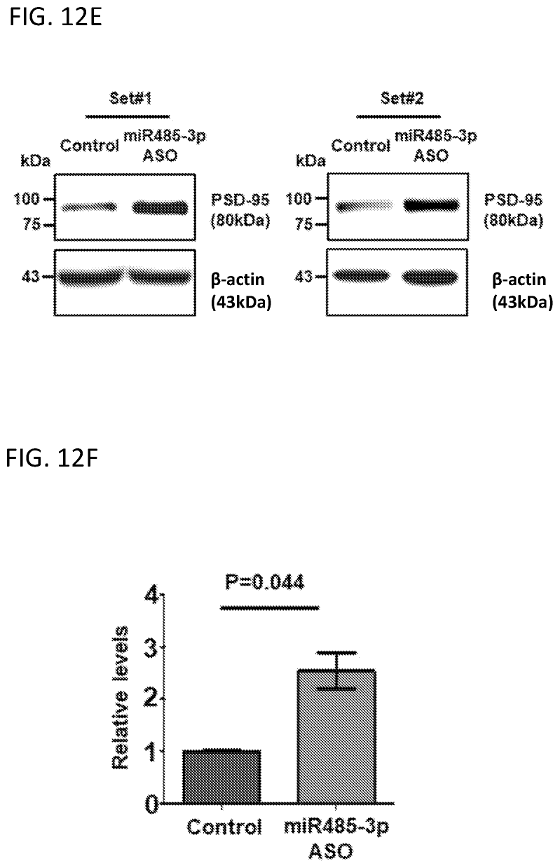

Based on western blot analysis, the expression of NeuN was increased, while the protein expression of caspase-3 was reduced, in the cortical region of miR-485 inhibitor treated animals (see FIGS. 12A and 12B). This effect, however, was not seen in the hippocampus under the same conditions. Similar results were observed using immunohistochemistry (see FIGS. 12C and 12D).

Next, the effect of miR-485 inhibitors on post-synapse was examined by assessing PSD-95 expression. As shown in FIGS. 12E and 12F, PSD-95 protein expression was significantly higher in the frontal cortex of AD mice treated with the miR-485 inhibitor, compared to the control animals.

The above results further demonstrate the therapeutic effects of the miR-485 inhibitors disclosed herein on AD by showing that the inhibitors can not only minimize neuronal loss but can also increase post-synapse.

### Example 14: Analysis of miR-485 Inhibitor on Cognitive Function

To determine whether the results observed above in Example 12 (i.e., increased post-synapse and reduced neuronal loss) are correlated with improvement in cognitive functions, AD mice were again treated with the miR-485 inhibitor or miR-control as described in the earlier examples. Then, cognitive functions were assessed in the animals using Y-maze and passive avoidance task (PAT), which are widely accepted as behavior paradigms for evaluating spatial working memory.

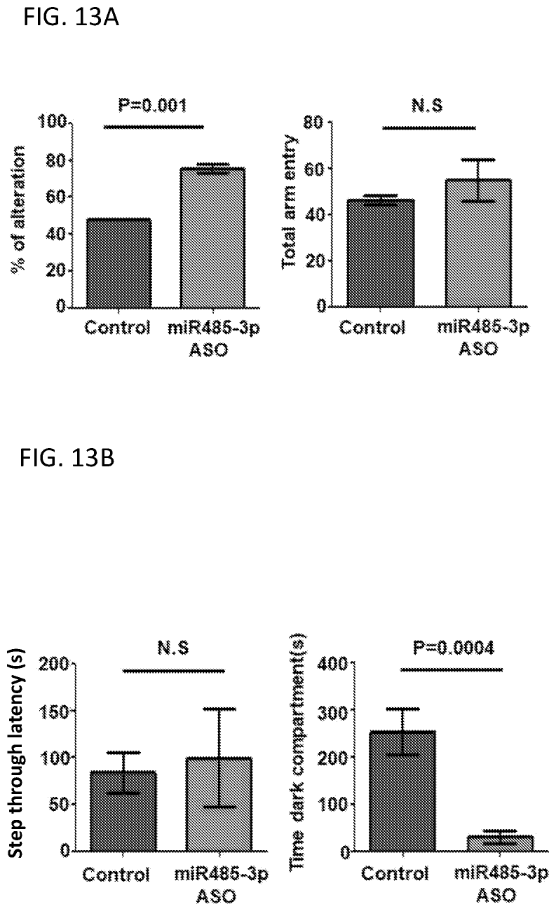

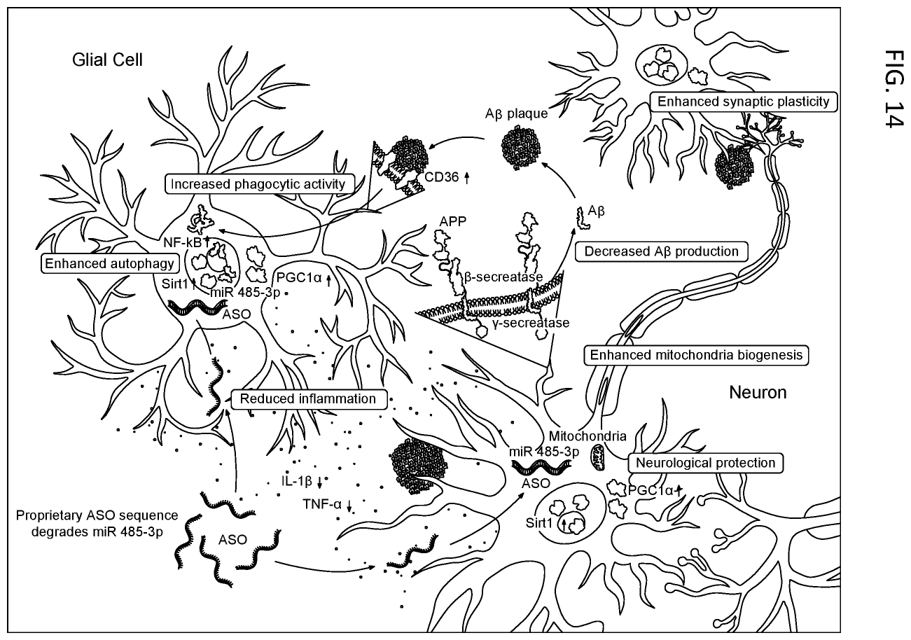

Two days after the last injection, we found that the spontaneous alternation percentage was significantly increased in miR-485 inhibitor treated mice. The total number of arm entries did not differ significantly between control and miR-485 inhibitor treated 5XFAD, indicating that levels of general motor and exploratory activity in the Y-maze were not changed (FIG. 13A). In addition, we examined associative memory in the passive avoidance task, based on the association formed between an electrical foot shock and a spontaneously preferred specific environmental context (darkness vs light). Step-through latency was similar between control and miR-485 inhibitor treated 5XFAD. However, miR-485 inhibitor treated mice showed a significant reduction in the latency to spend time the dark compartment 24 hr after receiving an electrical shock (FIG. 13B).

The above results collectively demonstrate that the miR-485 inhibitors disclosed herein can regulate (i.e., increase) the expression of different genes involved in neurodegenerative diseases, such as AD. As shown in the above Examples, such genes include SIRT1, CD36, and PGC-1α. Not to be bound by any one theory, the above results show that by regulating the expression of these genes, miR-485 inhibitors disclosed herein can treat many aspects of AD (e.g., reduce both Aβ production and plaque formation, promote Aβ plaque phagocytosis, reduce neuroinflammation, reduce neuronal loss, increase post-synapse, and improve cognitive functions) (see FIG. 14).

### Example 15: Analysis of the Potency of miR-485 Inhibitors in Regulating SIRT1, PGC-1α, and CD36 Expression In Vivo

To further assess the potency of miR-485 inhibitors disclosed herein in regulating the expression of SIRT1, PGC-1α, and CD36, a single dose (100 μg/mouse; 5 mg/kg) of the miR-485 inhibitor (see Example 1) was administered (via intraventricular administration) to wild-type male Crl:CD1 (ICR) mice, which were purchased from KOATECH (Korea). Control animals received the miR-control (see Example 1). Then, the animals were sacrificed at various time points post-administration, and the expression level of SIRT1, PGC-1α, and CD36 was assessed in both the cortex and hippocampus of the brain using Western blot. Expression level of SIRT1 and PGC-1α was also measured in the serum using ELISA.

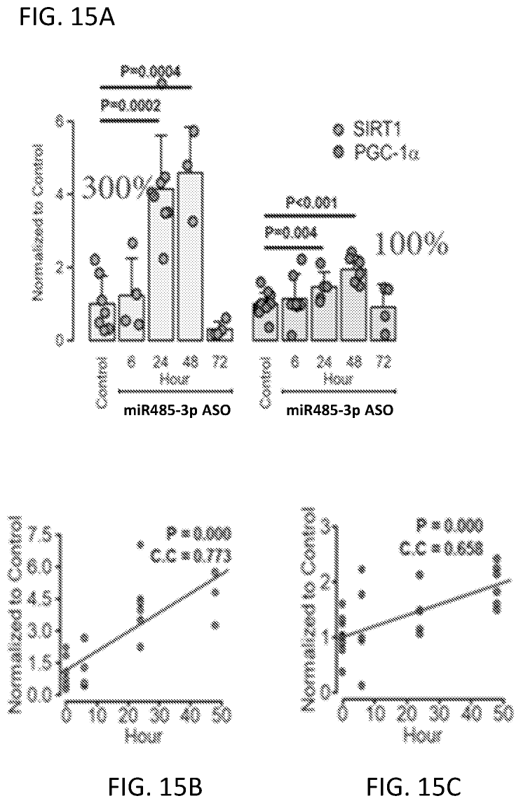

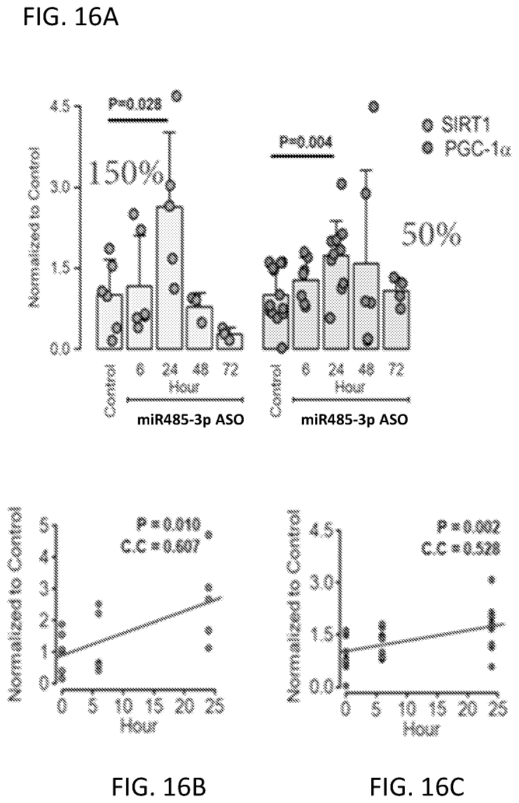

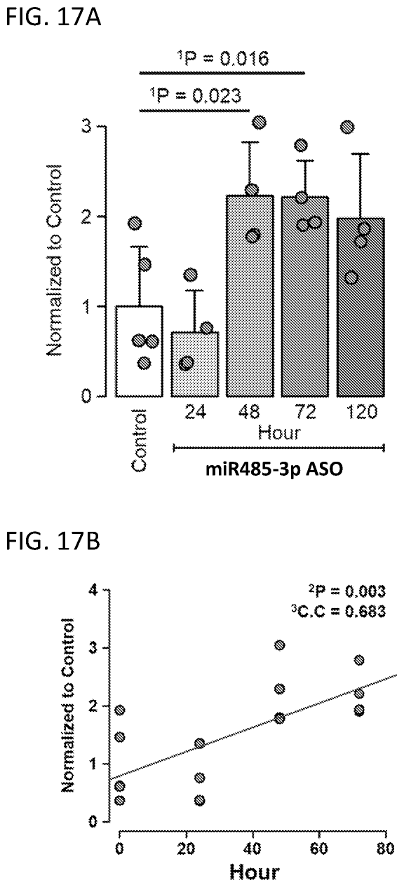

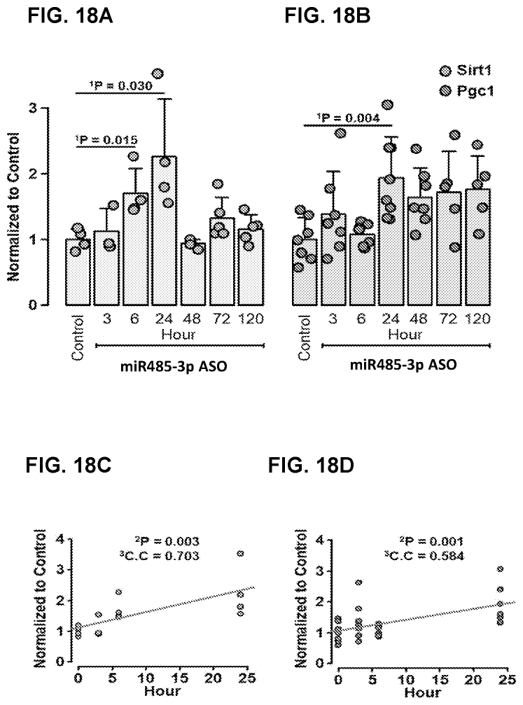

As shown in FIGS. 15A-15C, 16A-16C, and 17A-17B, a single administration of the miR-485 inhibitor resulted in rapid increase in SIRT1, PGC-1α, and CD36 expression in both the cortex and the hippocampus. For SIRT1, peak expression was observed in the cortex at about 48 hours post-administration (approximately 300% increase over the expression in control animals) and in the hippocampus at about 24 hours post-administration (approximately 150% increase over the control) (see FIGS. 15A and 16A, respectively). The peak expression for PGC-1α was also observed at about 48 hours post-administration in the cortex (approximately 100% increase over the control) and at about 24 hours post-administration in the hippocampus (approximately 50% increase over the control) (see FIGS. 15B and 16B, respectively). Similar results were observed for CD36 (see FIG. 17A). In the serum, single administration of the miR-485 inhibitor also resulted in increased expression of both SIRT1 (see FIGS. 18A and 18C) and PGC-1α (see FIGS. 18B and 18D). The overall expression pattern was similar to that observed in the brain.

The results confirm the potency of the miR-485 inhibitors disclosed herein in regulating SIRT1, PGC-1α, and CD36 expression. For comparison, a small molecule ApoE4 has previously been shown to have positive effects on normalizing SIRT1 expression in vivo. See Campagna et al., Sci Rep 8(1):17574 (December 2018). After 56 days of daily administration (at a dose of 40 mg/kg per day), there was approximately a 20% increase in SIRT1 expression in the hippocampus but no increase in the cortex. With the miR-485 inhibitor (e.g., SEQ ID NO: 28) disclosed herein, a single administration at a much lower dose (i.e., 5 mg/kg) resulted in significantly greater SIRT1 expression both in the hippocampus and the cortex. The results further suggest that by measuring, e.g., SIRT1 in the serum, it is possible to understand the expression level of, e.g., SIRT1 in the brain.

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

The contents of all cited references (including literature references, patents, patent applications, and websites) that can be cited throughout this application are hereby expressly incorporated by reference in their entirety for any purpose, as are the references cited therein.

What is claimed is:

1. A method of identifying a subject responsive to a miR-485 inhibitor therapy comprising measuring a level of a SIRT1 protein and/or a SIRT1 gene in the subject.

2. The method of claim 1, wherein the subject is previously administered a compound that inhibits miR-485 (miRNA inhibitor).

3. The method of claim 1, further comprising administering a compound that inhibits miR-485 (miRNA inhibitor).

4. The method of any one of claims 1 to 3, wherein the subject has a disease or a condition associated with a decreased level of a SIRT1 protein and/or a SIRT1 gene.

5. The method of any one of claims 1 to 4, wherein the miRNA inhibitor induces autophagy and/or treats or prevents inflammation.

6. A method of treating a disease or condition associated with an abnormal level of a SIRT1 protein and/or a SIRT1 gene in a subject in need thereof comprising administering to the subject a compound that inhibits miR-485 (miRNA inhibitor) and measuring a level of a SIRT1 protein and/or a SIRT1 gene in the subject.

7. The method of claim 6, wherein the level of the SIRT1 protein and/or SIRT1 gene is increased after the administration.

8. The method of claim 7, further comprises administering a second dose of the miRNA inhibitor to the subject.

9. The method of any one of claims 1 to 8, wherein the level of a SIRT1 protein and/or a SIRT1 gene in the subject is increased at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% in a frontal cortex section compared to the level prior to the administration.

10. The method of any one of claims 1 to 8, wherein the level of a SIRT1 protein and/or a SIRT1 gene in the subject is increased at least about 30%, at least about 40%, at least about 50%, at least about 60%, at least about 70%, at least about 80%, at least about 90%, at least about 100%, at least about 110%, at least about 120%, at least about 130%, at least about 140%, or at least about 150% in a hippocampus section compared to the level prior to the administration.

11. The method of any one of claims 1 to 8, wherein the level of a SIRT1 protein and/or a SIRT1 gene in the subject is increased at least about 30%, at least about 40%, at least about 50%, at least about 60%, at least about 70%, at least about 80%, at least about 90%, or at least about 100% in serum compared to the level prior to the administration.

12. The method of claims 9 to 11, wherein the level of a SIRT1 protein and/or a SIRT1 gene in the subject is measured within about 12 hours, about 24 hours, about 36 hours, or about 48 hours.

13. The method of any one of claims 1 to 12, wherein the measuring is in serum of the subject.

14. The method of claim 13, wherein the serum is collected after the administration.

15. The method of any one of claims 1 to 12, wherein the measuring is in the Cerebrospinal fluid (CSF) of the subject.

16. The method of any one of claims 1 to 15, wherein the miRNA inhibitor inhibits miR485-3p.

17. The method of claim 16, wherein the miR485-3p comprises 5′-gucauacacggcucuccucucu-3′ (SEQ ID NO: 1).

18. The method of any one of claims 1 to 17, wherein the miRNA inhibitor comprises a nucleotide sequence comprising 5′-UGUAUGA-3′ (SEQ ID NO: 2) and wherein the miRNA inhibitor comprises about 6 to about 30 nucleotides in length.

19. The method of any one of claims 1 to 18, wherein the miRNA inhibitor increases transcription of an SIRT1 gene and/or expression of a SIRT1 protein.

20. The method of any one of claims 1 to 19, wherein the miRNA inhibitor comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 5′ of the nucleotide sequence.

21. The method of any one of claims 1 to 20, wherein the miRNA inhibitor comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 3′ of the nucleotide sequence.

22. The method of any one of claims 1 to 21, wherein the miRNA inhibitor has a sequence selected from the group consisting of: 5′-UGUAUGA-3′ (SEQ ID NO: 2), 5′-GUGUAUGA-3′ (SEQ ID NO: 3), 5′-CGUGUAUGA-3′ (SEQ ID NO: 4), 5′-CCGUGUAUGA-3′ (SEQ ID NO: 5), 5′-GCCGUGUAUGA-3′ (SEQ ID NO: 6), 5′-AGCCGUGUAUGA-3′ (SEQ ID NO: 7), 5′-GAGCCGUGUAUGA-3′ (SEQ ID NO: 8), 5′-AGAGCCGUGUAUGA-3′ (SEQ ID NO: 9), 5′-GAGAGCCGUGUAUGA-3′ (SEQ ID NO: 10), 5′-GGAGAGCCGUGUAUGA-3′ (SEQ ID NO: 11), 5′-AGGAGAGCCGUGUAUGA-3′ (SEQ ID NO: 12), 5′-GAGGAGAGCCGUGUAUGA-3′ (SEQ ID NO: 13), 5′-AGAGGAGAGCCGUGUAUGA-3′ (SEQ ID NO: 14), 5′-GAGAGGAGAGCCGUGUAUGA-3′ (SEQ ID NO: 15); 5′-UGUAUGAC-3′ (SEQ ID NO: 16), 5′-GUGUAUGAC-3′ (SEQ ID NO: 17), 5′-CGUGUAUGAC-3′ (SEQ ID NO: 18), 5′-CCGUGUAUGAC-3′ (SEQ ID NO: 19), 5′-GCCGUGUAUGAC-3′ (SEQ ID NO: 20), 5′-AGCCGUGUAUGAC-3′ (SEQ ID NO: 21), 5′-GAGCCGUGUAUGAC-3′ (SEQ ID NO: 22), 5′-AGAGCCGUGUAUGAC-3′ (SEQ ID NO: 23), 5′-GAGAGCCGUGUAUGAC-3′ (SEQ ID NO: 24), 5′-GGAGAGCCGUGUAUGAC-3′ (SEQ ID NO: 25), 5′-AGGAGAGCCGUGUAUGAC-3′ (SEQ ID NO: 26), 5′-GAGGAGAGCCGUGUAUGAC-3′ (SEQ ID NO: 27), 5′-AGAGGAGAGCCGUGUAUGAC-3′ (SEQ ID NO: 28), 5′-GAGAGGAGAGCCGUGUAUGAC-3′ (SEQ ID NO: 29), or AGAGAGGAGAGCCGUGUAUGAC (SEQ ID NO: 30).

23. The method of any one of claims 1 to 21, wherein the miRNA inhibitor has a sequence selected from the group consisting of: 5′-TGTATGA-3′ (SEQ ID NO: 62), 5′-GTGTATGA-3′ (SEQ ID NO: 63), 5′-CGTGTATGA-3′ (SEQ ID NO: 64), 5′-CCGTGTATGA-3′ (SEQ ID NO: 65), 5′-GCCGTGTATGA-3′ (SEQ ID NO: 66), 5′-AGCCGTGTATGA-3′ (SEQ ID NO: 67), 5′-GAGCCGTGTATGA-3′ (SEQ ID NO: 68), 5′-AGAGCCGTGTATGA-3′ (SEQ ID NO: 69), 5′-GAGAGCCGTGTATGA-3′ (SEQ ID NO: 70), 5′-GGAGAGCCGTGTATGA-3′ (SEQ ID NO: 71), 5′-AGGAGAGCCGTGTATGA-3′ (SEQ ID NO: 72), 5′-GAGGAGAGCCGTGTATGA-3′ (SEQ ID NO: 73), 5′-AGAGGAGAGCCGTGTATGA-3′ (SEQ ID NO: 74), 5′-GAGAGGAGAGCCGTGTATGA-3′ (SEQ ID NO: 75); 5′-TGTATGAC-3′ (SEQ ID NO: 76), 5′-GTGTATGAC-3′ (SEQ ID NO: 77), 5′-CGTGTATGAC-3′ (SEQ ID NO: 78), 5′-CCGTGTATGAC-3′ (SEQ ID NO: 79), 5′-GCCGTGTATGAC-3′ (SEQ ID NO: 80), 5′-AGCCGTGTATGAC-3′ (SEQ ID NO: 81), 5′-GAGCCGTGTATGAC-3′ (SEQ ID NO: 82), 5′-AGAGCCGTGTATGAC-3′ (SEQ ID NO: 83), 5′-GAGAGCCGTGTATGAC-3′ (SEQ ID NO: 84), 5′-GGAGAGCCGTGTATGAC-3′ (SEQ ID NO: 85), 5′-AGGAGAGCCGTGTATGAC-3′ (SEQ ID NO: 86), 5′-GAGGAGAGCCGTGTATGAC-3′ (SEQ ID NO: 87), 5′-AGAGGAGAGCCGTGTATGAC-3′ (SEQ ID NO: 88), 5′-GAGAGGAGAGCCGTGTATGAC-3′ (SEQ ID NO: 89), and 5′-AGAGAGGAGAGCCGTGTATGAC-3′ (SEQ ID NO: 90).

24. The method of any one of claims 1 to 21, wherein the sequence of the miRNA inhibitor is at least about 50%, at least about 55%, at least about 60%, at least about 65%, at least about 70%, at least about 75%, at least about 80%, at least about 85%, at least about 90%, or at least about 95% sequence identity to 5′-AGAGAGGAGAGCCGUGUAUGAC-3′ (SEQ ID NO: 30) or 5′-AGAGAGGAGAGCCGTGTATGAC-3′ (SEQ ID NO: 90).

25. The method of claim 24, wherein the miRNA inhibitor has a sequence that has at least 90% similarity to 5′-AGAGAGGAGAGCCGUGUAUGAC-3′ (SEQ ID NO: 30) or 5′-AGAGAGGAGAGCCGTGTATGAC-3′ (SEQ ID NO: 90).

26. The method of any one of claims 1 to 24, wherein the miRNA inhibitor comprises the nucleotide sequence 5′-AGAGAGGAGAGCCGUGUAUGAC-3′ (SEQ ID NO: 30) or 5′-AGAGAGGAGAGCCGTGTATGAC-3′ (SEQ ID NO: 90) with one substitution or two substitutions.

27. The method of any one of claims 1 to 24, wherein the miRNA inhibitor comprises the nucleotide sequence 5′-AGAGAGGAGAGCCGUGUAUGAC-3′ (SEQ ID NO: 30) or 5′-AGAGAGGAGAGCCGTGTATGAC-3′ (SEQ ID NO: 90).

28. The method of claim 27, wherein the miRNA inhibitor comprises the nucleotide sequence 5′-AGAGAGGAGAGCCGUGUAUGAC-3′ (SEQ ID NO: 30).

29. The method of any one of claims 1 to 28, wherein the miRNA inhibitor comprises at least one modified nucleotide.

30. The method of claim 29, wherein the at least one modified nucleotide is a locked nucleic acid (LNA), an unlocked nucleic acid (UNA), an arabino nucleic acid (ABA), a bridged nucleic acid (BNA), and/or a peptide nucleic acid (PNA).

31. The method of any one of claims 1 to 30, wherein the miRNA inhibitor comprises a backbone modification.

32. The method of claim 31, wherein the backbone modification is a phosphorodiamidate morpholino oligomer (PMO) and/or phosphorothioate (PS) modification.

33. The method of any one of claims 1 to 32, wherein the miRNA inhibitor is delivered in a delivery agent.

34. The method of claim 33, wherein the delivery agent comprises a micelle, an exosome, a lipidoid, a liposome, a lipoplex, a lipid nanoparticle, an extracellular vesicle, a synthetic vesicle, a polymeric compound, a peptide, a protein, a cell, a nanoparticle mimic, a nanotube, a conjugate, a viral vector, or combinations thereof.

35. The method of claim 33 or 34, wherein the delivery agent comprises a cationic carrier unit comprising
[WP]-L1-[CC]-L2-[AM] (formula I)
or
[WP]-L1-[AM]-L2-[CC] (formula II)
wherein
WP is a water-soluble biopolymer moiety;
CC is a cationic carrier moiety;
AM is an adjuvant moiety; and,
L1 and L2 are independently optional linkers.

36. The method of claim 35, wherein the miRNA inhibitor and the cationic carrier unit are capable of associating with each other to form a micelle when mixed together.

37. The method of claim 36, wherein the association is via a covalent bond.

38. The method of claim 36, wherein the association is via a non-covalent bond.

39. The method of claim 38, wherein the non-covalent bond comprises an ionic bond.

40. The method of any one of claims 35 to 39, wherein the water-soluble polymer comprises poly(alkylene glycols), poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyglycerol, polyphosphazene, polyoxazolines (“POZ”) poly(N-acryloylmorpholine), or any combinations thereof.

41. The method of any one of claims 35 to 40, wherein the water-soluble polymer comprises polyethylene glycol (“PEG”), polyglycerol, or poly(propylene glycol) (“PPG”).

42. The method of any one of claims 35 to 41, wherein the water-soluble polymer comprises: 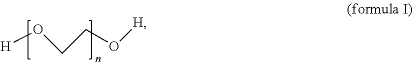
wherein n is 1-1000.

43. The method of claim 42, wherein the n is at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, or at least about 141.

44. The method of claim 42, wherein the n is about 80 to about 90, about 90 to about 100, about 100 to about 110, about 110 to about 120, about 120 to about 130, about 140 to about 150, about 150 to about 160.

45. The method of any one of claims 35 to 44, wherein the water-soluble polymer is linear, branched, or dendritic.

46. The method of any one of claims 35 to 45, wherein the cationic carrier moiety comprises one or more basic amino acids.

47. The method of claim 46, wherein the cationic carrier moiety comprises at least three, at least four, at least five, at least six, at least seven, at least eight, at least nine, at least ten, at least 11, at least 12, at least 13, at least 14, at last 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 basic amino acids.

48. The method of claim 47, wherein the cationic carrier moiety comprises about 30 to about 50 basic amino acids.

49. The method of claim 47 or 48, wherein the basic amino acid comprises arginine, lysine, histidine, or any combination thereof.

50. The method of any one of claims 35 to 49, wherein the cationic carrier moiety comprises about 40 lysine monomers.

51. The method of any one of claims 35 to 50, wherein the adjuvant moiety is capable of modulating an immune response, an inflammatory response, and/or a tissue microenvironment.

52. The method of any one of claims 35 to 51, wherein the adjuvant moiety comprises an imidazole derivative, an amino acid, a vitamin, or any combination thereof.

53. The composition of claim 52, wherein the adjuvant moiety comprises: 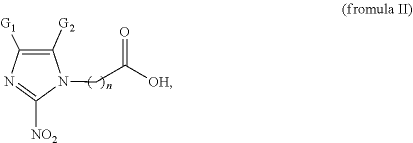
wherein each of G1 and G2 is H, an aromatic ring, or 1-10 alkyl, or G1 and G2 together form an aromatic ring, and wherein n is 1-10.

54. The method of claim 52, wherein the adjuvant moiety comprises nitroimidazole.

55. The method of claim 52, wherein the adjuvant moiety comprises metronidazole, tinidazole, nimorazole, dimetridazole, pretomanid, ornidazole, megazol, azanidazole, benznidazole, or any combination thereof.

56. The method of any one of claims 35 to 52, wherein the adjuvant moiety comprises an amino acid.

57. The method of claim 56, wherein the adjuvant moiety comprises 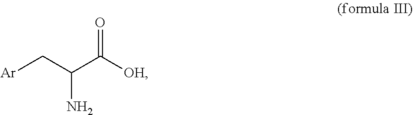
wherein Ar is 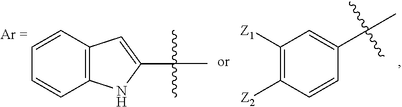
and
wherein each of Z1 and Z2 is H or OH.

58. The method of any one of claims 35 to 51, wherein the adjuvant moiety comprises a vitamin.

59. The method of claim 58, wherein the vitamin comprises a cyclic ring or cyclic hetero atom ring and a carboxyl group or hydroxyl group.

60. The method of claim 58 or claim 59, wherein the vitamin comprises: 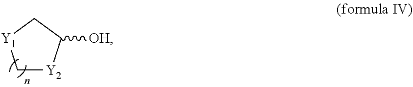
wherein each of Y1 and Y2 is C, N, O, or S, and wherein n is 1 or 2.

61. The method of any one of claims 58 to 60, wherein the vitamin is selected from the group consisting of vitamin A, vitamin B1, vitamin B2, vitamin B3, vitamin B6, vitamin B7, vitamin B9, vitamin B12, vitamin C, vitamin D2, vitamin D3, vitamin E, vitamin M, vitamin H, and any combination thereof.

62. The method of any one of claims 58 to 61, wherein the vitamin is vitamin B3.

63. The method of any one of claims 58 to 62, wherein the adjuvant moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, or at least about 20 vitamin B3.

64. The method of claim 63, wherein the adjuvant moiety comprises about 10 vitamin B3.

65. The method of any one of claims 58 to 64, wherein the delivery agent comprises a water-soluble biopolymer moiety with about 120 to about 130 PEG units, a cationic carrier moiety comprising a poly-lysine with about 30 to about 40 lysines, and an adjuvant moiety with about 5 to about 10 vitamin B3.

66. The method of any one of claims 35 to 65, wherein the cationic carrier unit is capable of protecting the miRNA inhibitor from enzymatic degradation.

67. The method of any one of claims 4 to 66, wherein the disease or condition comprises Alzheimer's disease.

68. The method of any one of claims 2 to 67, wherein the miRNA inhibitor is administered intranasally, parenthetically, intramuscularly, subcutaneously, ophthalmic, intravenously, intraperitoneally, intradermally, intraorbitally, intracerebrally, intracranially, intracerebroventricularly, intraspinally, intraventricular, intrathecally, intracistemally, intracapsularly, intratumorally, topically, or any combination thereof.

69. The method of any one of claims 4 to 66 and 68, wherein the disease or condition comprises autism spectrum disorder, mental retardation, seizure, stroke, Parkinson's disease, spinal cord injury, or combinations thereof.

70. The method of claim 33, wherein the delivery agent is a micelle.

71. The method of claim 70, wherein the micelle comprises (i) about 100 to about 200 PEG units, (ii) about 30 to about 40 lysines, each with an amine group, (iii) about 15 to about 20 lysines, each with a thiol group, and (iv) about 30 to about 40 lysines, each linked to vitamin B3.

72. The method of claim 70, wherein the micelle comprises (i) about 120 to about 130 PEG units, (ii) about 32 lysines, each with an amine group, (iii) about 16 lysines, each with a thiol group, and (iv) about 32 lysines, each linked to vitamin B3.

73. The method of claim 71 or 72, wherein a targeting moiety is further linked to the PEG units.

74. The method of claim 73, wherein the targeting moiety is a LAT 1 targeting ligand.

75. The method of claim 73, wherein the targeting moiety is phenyl alanine.


SEQUENCE LISTING (93 sequences)

SEQ ID NO: 1 - RNA, 22 nt, Artificial Sequence
  oligonucleotide
gucauacacggcucuccucucu

SEQ ID NO: 2 - RNA, 7 nt, Artificial Sequence
  oligonucleotide
uguauga

SEQ ID NO: 3 - RNA, 8 nt, Artificial Sequence
  oligonucleotide
guguauga

SEQ ID NO: 4 - RNA, 9 nt, Artificial Sequence
  oligonucleotide
cguguauga

SEQ ID NO: 5 - RNA, 10 nt, Artificial Sequence
  oligonucleotide
ccguguauga

SEQ ID NO: 6 - RNA, 11 nt, Artificial Sequence
  oligonucleotide
gccguguauga

SEQ ID NO: 7 - RNA, 12 nt, Artificial Sequence
  oligonucleotide
agccguguauga

SEQ ID NO: 8 - RNA, 13 nt, Artificial Sequence
  oligonucleotide
gagccguguauga

SEQ ID NO: 9 - RNA, 14 nt, Artificial Sequence
  oligonucleotide
agagccguguauga

SEQ ID NO: 10 - RNA, 15 nt, Artificial Sequence
  oligonucleotide
gagagccguguauga

SEQ ID NO: 11 - RNA, 16 nt, Artificial Sequence
  oligonucleotide
ggagagccguguauga

SEQ ID NO: 12 - RNA, 17 nt, Artificial Sequence
  oligonucleotide
aggagagccguguauga

SEQ ID NO: 13 - RNA, 18 nt, Artificial Sequence
  oligonucleotide
gaggagagccguguauga

SEQ ID NO: 14 - RNA, 19 nt, Artificial Sequence
  oligonucleotide
agaggagagccguguauga

SEQ ID NO: 15 - RNA, 20 nt, Artificial Sequence
  oligonucleotide
gagaggagagccguguauga

SEQ ID NO: 16 - RNA, 8 nt, Artificial Sequence
  oligonucleotide
uguaugac

SEQ ID NO: 17 - RNA, 9 nt, Artificial Sequence
  oligonucleotide
guguaugac

SEQ ID NO: 18 - RNA, 10 nt, Artificial Sequence
  oligonucleotide
cguguaugac

SEQ ID NO: 19 - RNA, 11 nt, Artificial Sequence
  oligonucleotide
ccguguaugac

SEQ ID NO: 20 - RNA, 12 nt, Artificial Sequence
  oligonucleotide
gccguguaugac

SEQ ID NO: 21 - RNA, 13 nt, Artificial Sequence
  oligonucleotide
agccguguaugac

SEQ ID NO: 22 - RNA, 14 nt, Artificial Sequence
  oligonucleotide
gagccguguaugac

SEQ ID NO: 23 - RNA, 15 nt, Artificial Sequence
  oligonucleotide
agagccguguaugac

SEQ ID NO: 24 - RNA, 16 nt, Artificial Sequence
  oligonucleotide
gagagccguguaugac

SEQ ID NO: 25 - RNA, 17 nt, Artificial Sequence
  oligonucleotide
ggagagccguguaugac

SEQ ID NO: 26 - RNA, 18 nt, Artificial Sequence
  oligonucleotide
aggagagccguguaugac

SEQ ID NO: 27 - RNA, 19 nt, Artificial Sequence
  oligonucleotide
gaggagagccguguaugac

SEQ ID NO: 28 - RNA, 20 nt, Artificial Sequence
  oligonucleotide
agaggagagccguguaugac

SEQ ID NO: 29 - RNA, 21 nt, Artificial Sequence
  oligonucleotide
gagaggagagccguguaugac

SEQ ID NO: 30 - RNA, 22 nt, Artificial Sequence
  oligonucleotide
agagaggagagccguguaugac

SEQ ID NO: 31 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 32 - protein, 561 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 33 - RNA, 22 nt, Homo sapiens
agaggcuggccgugaugaauuc

SEQ ID NO: 34 - RNA, 22 nt, Artificial Sequence
  miR-485-3p
agucauacacggcucuccucuc

SEQ ID NO: 35 - RNA, 22 nt, Artificial Sequence
  miR-485-5p
agaggcuggccgugaugaauuc

SEQ ID NO: 36 - protein, 472 aa, Homo sapiens
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 37 - protein, 288 aa, Homo sapiens
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRETCVHFTSSFSVCKS

SEQ ID NO: 38 - protein, 433 aa, Homo sapiens
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 39 - protein, 412 aa, Homo sapiens
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAYNNTADGVYKVFNGKDNISKVAIIDTYKGKRNLSYWESHCDMINGTDAASFPPFVEKSQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 40 - protein, 798 aa, Homo sapiens
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 41 - protein, 271 aa, Homo sapiens
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNLFL

SEQ ID NO: 42 - protein, 803 aa, Homo sapiens
MDETSPRLEEDWKKVLQREAGWQCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 43 - protein, 786 aa, Homo sapiens
MDEGYFCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 44 - protein, 289 aa, Homo sapiens
MDEGYFCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGVKTNLISK

SEQ ID NO: 45 - protein, 276 aa, Homo sapiens
MDETSPRLEEDWKKVLQREAGWQCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNLFL

SEQ ID NO: 46 - protein, 138 aa, Homo sapiens
MDEGYFCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLVRTLPTV

SEQ ID NO: 47 - protein, 301 aa, Homo sapiens
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGVKTNLISK

SEQ ID NO: 48 - protein, 671 aa, Homo sapiens
MPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 49 - RNA, 7 nt, Artificial Sequence
  oligonucleotide
ucauaca

SEQ ID NO: 50 - RNA, 7 nt, Artificial Sequence
  oligonucleotide
gaggcug

SEQ ID NO: 51 - DNA, 18 bp, Artificial Sequence
  oligonucleotide
cgaggtcgacttcctaga

SEQ ID NO: 52 - DNA, 23 bp, Artificial Sequence
  oligonucleotide
catacacggctctcctctctaaa

SEQ ID NO: 53 - DNA, 20 bp, Artificial Sequence
  oligonucleotide
tcctgtggcatccatgaaac

SEQ ID NO: 54 - DNA, 20 bp, Artificial Sequence
  oligonucleotide
caatgcctgggtacatggtg

SEQ ID NO: 55 - DNA, 18 bp, Artificial Sequence
  oligonucleotide
ccaagtggaggagcagct

SEQ ID NO: 56 - DNA, 20 bp, Artificial Sequence
  oligonucleotide
gacaaggtacaacccatcgg

SEQ ID NO: 57 - DNA, 22 bp, Artificial Sequence
  oligonucleotide
ttcgacacatgggataacgagg

SEQ ID NO: 58 - DNA, 21 bp, Artificial Sequence
  oligonucleotide
tttttgctgtgagtcccggag

SEQ ID NO: 59 - DNA, 20 bp, Artificial Sequence
  oligonucleotide
cagcctagcagcacgtaaat

SEQ ID NO: 60 - DNA, 19 bp, Artificial Sequence
  oligonucleotide
gaatcgagcaccagttacg

SEQ ID NO: 61 - DNA, 22 bp, Artificial Sequence
  oligonucleotide
ccttccctgaaggttcctcctt

SEQ ID NO: 62 - DNA, 7 bp, artificial Sequence
  oligonucleotide
tgtatga

SEQ ID NO: 63 - DNA, 8 bp, artificial Sequence
  oligonucleotide
gtgtatga

SEQ ID NO: 64 - DNA, 9 bp, artificial Sequence
  oligonucleotide
cgtgtatga

SEQ ID NO: 65 - DNA, 10 bp, artificial Sequence
  oligonucleotide
ccgtgtatga

SEQ ID NO: 66 - DNA, 11 bp, artificial Sequence
  oligonucleotide
gccgtgtatga

SEQ ID NO: 67 - DNA, 12 bp, artificial Sequence
  oligonucleotide
agccgtgtatga

SEQ ID NO: 68 - DNA, 13 bp, artificial Sequence
  oligonucleotide
gagccgtgtatga

SEQ ID NO: 69 - DNA, 14 bp, artificial Sequence
  oligonucleotide
agagccgtgtatga

SEQ ID NO: 70 - DNA, 15 bp, artificial Sequence
  oligonucleotide
gagagccgtgtatga

SEQ ID NO: 71 - DNA, 16 bp, artificial Sequence
  oligonucleotide
ggagagccgtgtatga

SEQ ID NO: 72 - DNA, 17 bp, artificial Sequence
  oligonucleotide
aggagagccgtgtatga

SEQ ID NO: 73 - DNA, 18 bp, artificial Sequence
  oligonucleotide
gaggagagccgtgtatga

SEQ ID NO: 74 - DNA, 19 bp, artificial Sequence
  oligonucleotide
agaggagagccgtgtatga

SEQ ID NO: 75 - DNA, 20 bp, artificial Sequence
  oligonucleotide
gagaggagagccgtgtatga

SEQ ID NO: 76 - DNA, 8 bp, artificial Sequence
  oligonucleotide
tgtatgac

SEQ ID NO: 77 - DNA, 9 bp, artificial Sequence
  oligonucleotide
gtgtatgac

SEQ ID NO: 78 - DNA, 10 bp, artificial Sequence
  oligonucleotide
cgtgtatgac

SEQ ID NO: 79 - DNA, 11 bp, artificial Sequence
  oligonucleotide
ccgtgtatgac

SEQ ID NO: 80 - DNA, 12 bp, artificial Sequence
  oligonucleotide
gccgtgtatgac

SEQ ID NO: 81 - DNA, 13 bp, artificial Sequence
  oligonucleotide
agccgtgtatgac

SEQ ID NO: 82 - DNA, 14 bp, artificial Sequence
  oligonucleotide
gagccgtgtatgac

SEQ ID NO: 83 - DNA, 15 bp, artificial Sequence
  oligonucleotide
agagccgtgtatgac

SEQ ID NO: 84 - DNA, 16 bp, artificial Sequence
  oligonucleotide
gagagccgtgtatgac

SEQ ID NO: 85 - DNA, 17 bp, artificial Sequence
  oligonucleotide
ggagagccgtgtatgac

SEQ ID NO: 86 - DNA, 18 bp, artificial Sequence
  oligonucleotide
aggagagccgtgtatgac

SEQ ID NO: 87 - DNA, 19 bp, artificial Sequence
  oligonucleotide
gaggagagccgtgtatgac

SEQ ID NO: 88 - DNA, 20 bp, artificial Sequence
  oligonucleotide
agaggagagccgtgtatgac

SEQ ID NO: 89 - DNA, 21 bp, artificial Sequence
  oligonucleotide
gagaggagagccgtgtatgac

SEQ ID NO: 90 - DNA, 22 bp, artificial Sequence
  oligonucleotide
agagaggagagccgtgtatgac

SEQ ID NO: 91 - DNA, 22 bp, Artificial Sequence
  Oligonucleotide
ucucuccucucggcacauacug

SEQ ID NO: 92 - DNA, 22 bp, Artificial Sequence
  Oligonucleotide
uuuaaagguucauuuguaugau

SEQ ID NO: 93 - DNA, 22 bp, Artificial Sequence
  Oligonucleotide
uuuaaagguucauuagatagtu